(19) United States
(12) Patent Application Publication
(10) Pub. No.: US 2022/0235384 A1
(43) Pub. Date: Jul. 28, 2022

(54) NANORG MICROBIAL FACTORIES: LIGHT-DRIVEN RENEWABLE BIOCHEMICAL SYNTHESIS USING QUANTUM DOT-BACTERIA NANO-BIOHYBRIDS

(71) Applicant: THE REGENTS OF THE UNIVERSITY OF COLORADO, A BODY CORPORATE, Denver, CO (US)

(72) Inventors: Prashant Nagpal, Lafayette, CO (US); Yuchen Ding, Boulder, CO (US); John Bertram, Broomfield, CO (US)

(21) Appl. No.: 17/611,109

(22) PCT Filed: May 14, 2020
(86) PCT No.: PCT/US20/32899

§ 371 Date: Nov. 12, 2021

Related U.S. Application Data
(60) Provisional application No. 62/927,452, filed on Oct. 29, 2019.
(60) Provisional application No. 62/847,653, filed on May 14, 2019.

(51) Int. Cl. C12P 7/625; C12N 1/20; C12N 1/38; C12P 3/00; C12N 15/74; C12N 9/02

(52) U.S. Cl. CPC C12P 7/625; C12N 1/20; C12N 1/38; C12P 3/00; C12R 2001/065; C12N 9/0095; C12Y 118/06001; C12N 2800/101; C12N 15/74

(57) ABSTRACT

The invention relates to a nano-biohybrid organism (or nanorg) comprising one of at least seven different core-shell quantum dots (QDs) or gold nanoparticle clusters, with excitations ranging from ultraviolet to near-infrared energies, couple with targeted enzyme sites in bacteria. When illuminated by light, these QDs drive the renewable production of biofuel molecules and chemicals using carbon-dioxide (CO₂), water, and nitrogen (from air) as substrates. Nanorgs catalyze light-induced air-water-CO₂ reduction with a high turnover number (TON) of approximately 10⁶-10⁸ (mols of product per mol of cells) to biofuels such as isopropanol (IPA), butane diol, gasoline additives, gasoline substitutes, 2,3-butanediol (BDO), C11-C15 methyl ketones (MKs), and hydrogen (H2); Sand chemicals such as formic acid (FA), ammonia (NH₃), ethylene (C₂H₄), and degradable bioplastics, e.g. polyhydroxybutyrate (PHB). These nanorg cells function as nano-microbial factories powered by light.

### GOVERNMENT SUPPORT

This invention was made with government support under grant number CBET1351281 awarded by the National Science Foundation. The government has certain rights in the invention.

### FIELD OF THE INVENTION

The invention relates to a nano-biohybrid (e.g., a nanoorg) organism (e.g. a bacteria) comprising different core-shell quantum dots (QDs) or gold nanoparticle clusters, with excitation ranges having ultraviolet to near-infrared energies, coupled with targeted enzyme sites. When illuminated by light, these nanorgs drive the renewable production of biofuel molecules and chemicals using carbon-dioxide (CO₂), water, and nitrogen (from air) as substrates. The disclosed nanorgs catalyze light-induced air-water-CO₂ reduction with a high turnover number (TON) of approximately 10⁶-10⁸ (mols of product per mol of cells) to biofuels including, but not limited to, isopropanol (IPA), butane diol, gasoline additives, gasoline substitutes, 2,3-butanediol (BDO), C11-C15 methyl ketones (MKs), and hydrogen (H2); and chemicals such as formic acid (FA), ammonia (NH₃), ethylene (C₂H₄), and degradable bioplastics, e.g. polyhydroxybutyrate (PHB). These nanoorg cells function as nano-microbial factories powered by light.

### BACKGROUND

Attempts were made to combine the desired functionality of direct light-activation in cell free extracts/purified enzymes (Reference No. 3) and whole non-photosynthetic bacteria (Reference No. 4), but these strategies have limited applicability due to either enzyme de-activation in the air, or specific tolerance of the bacteria to inorganic elements.

There has been intensive searches for new methods to combine multiple functionalities (e.g., light, voltage, or magnetic field stimulation) of inorganic nanomaterials with the versatility of designed synthetic metabolic networks in living cells, to simply “grow” such hybrid catalysts by the addition of inorganic nanomaterials to the media/buffered water, thereby combining specificity of biocatalysts with high-throughput of inorganic nanomaterials.

However, living cells do not interface naturally with nanoscale materials.

Because such artificial organisms are contemplated to have unprecedented multifunctional properties, such as wireless activation of enzyme function using electromagnetic stimuli, there is a need for new artificial organisms.

### SUMMARY OF THE INVENTION

The invention relates to a nano-biohybrid (e.g., a nanoorg) organism (e.g. a bacteria) comprising different core-shell quantum dots (QDs) or gold nanoparticle clusters, with excitation ranges having ultraviolet to near-infrared energies, coupled with targeted enzyme sites. When illuminated by light, these nanorgs drive the renewable production of biofuel molecules and chemicals using carbon-dioxide (CO₂), water, and nitrogen (from air) as substrates. The disclosed nanorgs catalyze light-induced air-water-CO₂ reduction with a high turnover number (TON) of approximately 10⁶-10⁸ (mols of product per mol of cells) to biofuels including, but not limited to, isopropanol (IPA), butane diol, gasoline additives, gasoline substitutes, 2,3-butanediol (BDO), C11-C15 methyl ketones (MKs), and hydrogen (H2); and chemicals such as formic acid (FA), ammonia (NH₃), ethylene (C₂H₄), and degradable bioplastics, e.g. polyhydroxybutyrate (PHB). These nanorg cells function as nano-microbial factories powered by light.

In one embodiment, the present invention provides a method of producing a biofuel, comprising, a) providing, i) a live engineered Cupriavidus necator bacteria comprising an exogenous protein enzyme that produces a biofuel molecule and a quantum dot that transmits electrons having energies in the range of the reduction potential of said enzyme upon exposure to radiation for boosting production of said biofuel molecule, ii) an illumination source for emitting radiation, and iii) compounds comprising CO₂, H₂O, O₂ and N₂, b) incubating said live engineered bacteria in the presence of said compounds in the dark, and c) illuminating said live engineered bacteria with said source for producing a biofuel molecule. In one embodiment, said biofuel is a biodiesel. In one embodiment, said biofuel is a methyl ketone (MK). In one embodiment, said biodiesel is a methyl ketone (MK). In one embodiment, said methyl ketone (MK) ranges from C₁₁-C₁₅. In one embodiment, said methyl ketone (MK) comprises C₁₁-C₁₅. In one embodiment, said biofuel is a biodiesel methyl ketone ranging from C₁₁-C₁₅. In one embodiment, said biofuel is a gasoline 2,3-butanediol (BDO) molecule. In one embodiment, said biofuel molecule is ethylene. In one embodiment, said biofuel molecule is isopropanol. In one embodiment, said biofuel molecule is an additive to fuel. In one embodiment, said biofuel molecule is butane diol. In one embodiment, said biofuel molecule is a gasoline additives. In one embodiment, said biofuel molecule is a gasoline substitute. In one embodiment, said illumination source is selected from the group consisting of a light bulb, a fluorescent bulb, a light-emitting diode (LED) and sunlight (e.g. solar radiation). In one embodiment, said boosting production is increasing production over said engineered bacteria that is not comprising said quantum dot.

In one embodiment, the present invention provides a core-shell quantum dot (QD), wherein said core is selected from the group consisting of cadmium sulfide (CdS), cadmium selenide (CdSe), indium phosphide (InP), and copper zinc tin sulfide (Cu₂ZnSnS₄) (CZTS), wherein said shell is a zinc sulfide (ZnS) shell. In one embodiment, said ZnS shell is a two monolayer shell. In one embodiment, said QD is selected from the group consisting of CZS1, CZS2, CZSe1, CZSe2, CZSe3, IPZS and CZTS. In one embodiment, said QD core has a zwitterion L-cysteine (CYS) ligand cap. In one embodiment, said QD core has a ligand cap selected from the group consisting of a 3-mercaptopropionic acid (MPA) capping ligand and a cysteamine (CA) ligand cap. In one embodiment, said QD has an excitation energy from wavelengths ranging from ultraviolet to near-infrared energies. In one embodiment, said QD has a range of emission energies after exposure to said excitation energies. In one embodiment, said QD further comprises a bacterium, wherein said bacterium expresses an enzyme which has a reduction potential energy range matching said QD emission energies.

In one embodiment, the present invention provides a method of using a core-shell quantum dot (QD), comprising, a) providing, i) a bacteria strain expressing an enzyme for producing a compound, said enzyme having a reduction potential in a range of energies, ii) a plurality of quantum dots (QDs) comprising core-shell quantum dot (QD), wherein said QD in turn comprises a core is selected from the group consisting of cadmium sulfide (CdS), cadmium selenide (CdSe), indium phosphide (InP), and copper zinc tin sulfide (Cu₂ZnSnS₄) (CZTS), wherein said a two monolayer zinc sulfide (ZnS) shell, and a zwitterion L-cysteine (CYS) ligand cap, wherein said QD emits energies within said range of reduction potential energies after illumination, ii) an illumination source capable of emitting radiation, and b) contacting said bacteria with said QDs such that said QDs are internalized by said bacteria after said ligand binding, and c) irradiating said QD contacted bacteria for providing said emission energies for boosting production amounts of said compound over amounts produced by said QD contacted bacteria prior to illumination. In one embodiment, said energy matched QDs increase enzyme activity of the energy-matched enzyme. In one embodiment, said illumination source emits radiation in wavelengths selected from the group consisting of near-UV, visible, NIR radiation, Light Emitting Diode (LED) and solar radiation. In one embodiment, said QD is selected from the group consisting of CZS1, CZS2, CZSe1, CZSe2, CZSe3, IPZS and CZTS. In one embodiment, said method further provides a solution, wherein said bacteria are added to said solution. In one embodiment, said solution is selected from the group consisting of buffer, growth media, and lysis solution. In one embodiment, said QD contacted bacteria are in said solution during step c), further comprising step d) precipitating said compound out of solution. In one embodiment, said lysis solution comprises a detergent. In one embodiment, said detergent is sodium dodecyl sulfate (SDS). In one embodiment, said method further provides a membrane for filtering said QD contacted bacteria out of solution, then step d), filtering said QD contacted bacteria out of solution. In one embodiment, said filtered QD contacted bacteria are suspended in said solution then repeating step c). In one embodiment, said filtered QD contacted bacteria are suspended in said buffer for harvesting said compounds from said illuminated QD contacted bacteria. In one embodiment, said method further provides a charged filtration membrane, wherein said filtered CZS-QD contacted bacteria are suspended in said lysis solution before or after step c), further comprising a step of filtering said lysed bacteria for recovering said QDs. In one embodiment, said method further comprises a step of using said recovered QDs as said plurality of QDs used in step b).

In one embodiment, the present invention provides a nanorg bacteria strain comprising a core-shell quantum dot (QD), wherein said QD in turn comprises a core is selected from the group consisting of cadmium sulfide (CdS), cadmium selenide (CdSe), indium phosphide (InP), and copper zinc tin sulfide (Cu₂ZnSnS₄) (CZTS), wherein said a two monolayer zinc sulfide (ZnS) shell, and a zwitterion L-cysteine (CYS) ligand cap.

In one embodiment, the present invention provides a composition comprising an Azobacteria vinelandii bacteria strain comprising a Cadmium sulfide (CdS) core and a zinc sulfide (ZnS) shell (CdS@ZnS) core-shell quantum dot (CZS-QD), and a molybdenum-iron nitrogenase (MFN) enzyme. In one embodiment, said bacteria contains ammonia (NH₃) molecules above natural levels. In one embodiment, said bacteria contains hydrogen (H₂) molecules above natural levels. In one embodiment, said amount is greater than 10⁵ moles of NH₃ per mole of bacteria cells. In one embodiment, said MFN enzyme is heterologous. In one embodiment, said bacteria comprises a cell lysate. In one embodiment, said bacteria are live bacteria. In one embodiment, said live bacteria are replicating. In one embodiment, said shell has two monolayers of zinc sulfide (ZnS). In one embodiment, said CZS-QD has a zwitterion L-cysteine (CYS) cap. In one embodiment, said strain is DJ995. In one embodiment, said composition further comprises CO₂/O₂ at a ratio of 4:1. In one embodiment, said composition further comprises a growth medium. In one embodiment, said growth medium comprises an amount of said NH₃ above levels for said bacteria cells without a CZS-QD. In one embodiment, said method further

In one embodiment, the present invention provides a composition comprising a Cupriavidus necator bacteria strain comprising a Cadmium sulfide core (CdS) and a zinc sulfide (ZnS) shell (CdS@ZnS) core-shell quantum dot and a molybdenum-iron nitrogenase (MFN) enzyme. In one embodiment, said bacteria contains ammonia (NH₃) above natural levels. In one embodiment, said bacteria contains hydrogen (H₂) molecules above natural levels. In one embodiment, said Cupriavidus necator strain is an engineered strain comprising a pBBRl-efe plasmid. In one embodiment, said MFN enzyme is heterologous to said bacteria strain.

In one embodiment, the present invention provides a method of producing ammonia (NH₃), comprising, a) providing, i) a bacteria strain comprising a molybdenum-iron nitrogenase (MFN) enzyme, wherein expression of said MFN enzyme results in ammonia (NH₃) formation, a plurality of quantum dots (QDs), wherein said QD is a Cadmium sulfide core and a zinc sulfide (ZnS) shell (CdS@ZnS) (CZS) core-shell quantum dot, wherein said QD transmits electrons having energies in the range of the reduction potential of said MFN enzyme upon exposure to radiation that increases the activity of said MFN enzyme, ii) an illumination source capable of emitting radiation, iii) at least one compound selected from the group consisting of CO₂, H₂O, O₂ and N₂, and b) incubating said engineered bacteria in the presence of said at least one compound in the dark, and c) irradiating said engineered bacteria with said illumination source under conditions that produce ammonia (NH₃). In one embodiment, said d production of NH₃ molecules is above natural levels. In one embodiment, said production of NH₃ is an amount greater than in said bacteria strain without said QD. In one embodiment, said production of NH₃ is an amount greater than in said bacteria strain without said irradiating. In one embodiment, said bacteria strain is an Azobacteria vinelandii bacteria strain. In one embodiment, said Azobacteria vinelandii strain is DJ995. In one embodiment, said bacteria strain is a Cupriavidus necator bacteria strain. In one embodiment, said Cupriavidus necator strain is an engineered strain comprising a pBBRl-efe plasmid. In one embodiment, said MFN enzyme is heterologous. In one embodiment, said method further provides a bacterial growth medium. In one embodiment, said bacteria are irradiated in said growth medium. In one embodiment, said NH₃ formation is in said growth medium. In one embodiment, said irradiation results in a yield amount greater than 10⁵ moles of NH₃ per mole of bacteria cells. In one embodiment, said bacteria comprise a cell lysate. In one embodiment, said bacteria are live bacteria. In one embodiment, said live bacteria are replicating. In one embodiment, said CZS-QD has two ZnS monolayers. In one embodiment, said CZS-QD has a zwitterion L-cysteine (CYS) ligand cap. In one embodiment, said radiation source emits wavelengths selected from the group consisting of near-UV, visible, NIR radiation, Light Emitting Diode (LED) and solar radiation. In one embodiment, said illumination source emits 400 nm radiation. In one embodiment, said illumination source emits yellow (near-infrared (NIR)) radiation. In one embodiment, said growth medium further comprises L-ascorbic acid. In one embodiment, said method further provides a growth medium further comprising 4-(2-hydroxyethyl)-1-piperazineethanesulfonic acid (HEPES) buffer.

In one embodiment, the present invention provides a composition comprising a Cupriavidus necator bacteria strain comprising a Cadmium sulfide (CdS) core and a zinc sulfide (ZnS) shell (CdS@ZnS) (CZS) core-shell quantum dot (QD), and a pBBRl-yfp expression plasmid. In one embodiment, said QD is CZS2.

In one embodiment, the present invention provides a composition comprising a Cupriavidus necator bacteria strain comprising a cadmium selenide (CdSe) core and a zinc sulfide (ZnS) shell (CdSe@ZnS) (CZSe) core-shell quantum dot (QD), a pBBRl-yfp expression plasmid. In one embodiment, said QD is CZSe3. In one embodiment, said bacteria contains Polyhydroxybutyrate (PHB) molecules in amounts greater than in said bacteria strain without said QD. In one embodiment, said bacteria contains Polyhydroxybutyrate (PHB) molecules above natural levels. In one embodiment, said PHB contains an amount greater than 10³ moles of PHB per mole of bacteria cells. In one embodiment, said Cupriavidus necator further comprises a H16+pBBR1-PphaC-YFP plasmid expressing an enzyme. In one embodiment, said Cupriavidus necator further comprises a pBBRl-yfp plasmid expressing a PHB enzyme sequence. In one embodiment, said enzyme sequence is codon optimized for expression in Cupriavidus necator. In one embodiment, said QD has a zwitterion L-cysteine (CYS) cap. In one embodiment, said QD has two ZnS monolayers. In one embodiment, said bacteria comprises a cell lysate. In one embodiment, said bacteria are live bacteria. In one embodiment, said live bacteria are replicating. In one embodiment, said illumination comprises 400 nm radiation. In one embodiment, said illumination comprises orange radiation (near-infrared (NIR)). In one embodiment, said composition further comprises CO₂/O₂ at a 4:1 ratio.

In one embodiment, the present invention provides a composition comprising a Azobacteria vinelandii bacteria strain comprising a core-shell quantum dot (QD) and Polyhydroxybutyrate (PHB) molecules. In one embodiment, said Polyhydroxybutyrate (PHB) molecules are in amounts greater than in said bacteria strain without said QD. In one embodiment, said Polyhydroxybutyrate (PHB) molecules are in amounts above natural levels.

In one embodiment, the present invention provides a method of producing Polyhydroxybutyrate (PHB), comprising, a) providing, i) a bacteria strain comprising a molybdenum-iron nitrogenase (MFN) enzyme, wherein expression of said MFN enzyme results in Polyhydroxybutyrate (PHB) formation, a plurality of core-shell quantum dots (QDs), wherein said QD transmits electrons having energies in the range of the reduction potential of said MFN enzyme upon exposure to radiation that increases the activity of said MFN enzyme, ii) an illumination source capable of emitting radiation, and iii) at least one compound selected from the group consisting of CO₂, H₂O, O₂ and N₂, b) incubating said engineered bacteria in the presence of said at least one compound in the dark, and c) irradiating said engineered bacteria with said illumination source under conditions that produce Polyhydroxybutyrate (PHB). In one embodiment, said QD is a cadmium sulfide (CdS) core zinc sulfide (ZnS) shell QD. In one embodiment, said QD is CZS2. In one embodiment, said QD is a cadmium selenide (CdSe core zinc sulfide (ZnS) shell QD. In one embodiment, said QD is CZSe3. In one embodiment, said QD has two ZnS monolayers. In one embodiment, said QD has a zwitterion L-cysteine (CYS) ligand cap. In one embodiment, said production of PHB molecules is above natural levels. In one embodiment, said production of PHB molecules is an amount greater than in said bacteria strain without said QD. In one embodiment, said production of PHB molecules is an amount greater than in said bacteria strain without said irradiation. In one embodiment, the method further provides bacteria growth medium, wherein said bacteria contact said growth medium. In one embodiment, said bacteria are illuminated when in contact with said growth medium. In one embodiment, said PHB molecules are secreted into said growth medium. In one embodiment, said greater amount is up to 150% greater. In one embodiment, said greater amount is up to 100 mg of said PHB per gram of bacteria cell dry weight (CDW). In one embodiment, said greater yield is obtained within a twenty-four hour illumination time. In one embodiment, said bacteria strain is a Cupriavidus necator bacteria strain. In one embodiment, said Cupriavidus necator strain is DJ995. In one embodiment, said bacteria strain is an Azobacteria vinelandii bacteria strain. In one embodiment, the method further comprises an extraction of PHB molecules with sodium hypochlorite/chloroform mixture. In one embodiment, the method further comprises the step of precipitating said PHB molecules with methanol/water from said chloroform extract. In one embodiment, the method further comprises the step of coagulating said PHB molecules by drying. In one embodiment, the method further comprises rehydrating PHB molecules in glacial acetic acid solution. In one embodiment, the method further comprises casting a PHB thin film from said rehydrated PHB molecules. In one embodiment, said radiation source emits wavelengths selected from the group consisting of near-UV, visible, NIR radiation, Light Emitting Diode (LED) and solar radiation. In one embodiment, said radiation source emits 400 nm radiation. In one embodiment, said radiation source emits orange (near-infrared (NIR)) radiation.

In one embodiment, the present invention provides a Cupriavidus necator bacteria comprising a CdS@ZnS (CZS) core-shell quantum dot (QD) and alcohol molecules. In one embodiment, said alcohol is selected from the group consisting of isopropanol, butane diol, gasoline additives and gasoline substitutes. In one embodiment, said bacteria contain said alcohol molecules. In one embodiment, said alcohol molecules are at a level greater than for said bacteria without a QD. In one embodiment, said composition further comprises a bacteria growth medium. In one embodiment, said alcohol molecules. In one embodiment, said growth medium contains said alcohol molecules at a level greater than for said bacteria without a QD. In one embodiment, said Cupriavidus necator bacteria comprises a H16_IPAl-10_int sequence. In one embodiment, said accumulated amounts ranging from 10⁶-10⁸ moles of isopropanol per mole of cells. In one embodiment, said bacteria comprise a cell lysate. In one embodiment, said bacteria are live bacteria. In one embodiment, said live bacteria are replicating. In one embodiment, said CZS-QD has two ZnS monolayers. In one embodiment, said CZS-QD has a zwitterion cysteine (CYS) cap. In one embodiment, the method further comprises 400 nm radiation. In one embodiment, the method further comprises red (near-infrared (NIR)) irradiation. In one embodiment, the method further comprises an irradiation source emitting wavelengths selected from the group consisting of near-UV, visible, and NIR irradiation. In one embodiment, said strain is DJ995. In one embodiment, the method further comprises a CO₂/O₂ 4:1 mixture.

In one embodiment, the present invention provides a composition comprising a live engineered Azobacteria vinelandii bacteria strain comprising a CdS@ZnS (CZS) core-shell quantum dot (QD) and alcohol molecules.

In one embodiment, the present invention provides a method of producing an alcohol molecule, comprising, a) providing, i) a Cupriavidus necator strain comprising an enzyme that produces an alcohol molecule and a quantum dot (QD), wherein said QD is a CdS@ZnS (CZS) core-shell quantum dot, that transmits electrons having energies in the range of the reduction potential of said enzyme upon exposure to radiation for boosting production of said molecule, ii) an illumination source capable of emitting radiation, and iii) compounds comprising CO₂, H₂O, O₂ and N₂, b) incubating said bacteria in the presence of said compounds in the dark, and c) illuminating said live engineered bacteria for illuminating said bacteria for producing alcohol molecules in a yield amount greater than in said bacteria strain without said QD. In one embodiment, said alcohol is selected from the group consisting of isopropanol, butane diol, gasoline additives and gasoline substitutes. In one embodiment, the method further provides a bacteria growth medium. In one embodiment, said bacteria contact said growth medium. In one embodiment, said bacteria are illuminated in contact with said growth medium. In one embodiment, said alcohol molecules are in said growth medium. In one embodiment, said illumination emits energies selected from the group consisting of near-UV, visible, NIR radiation, Light Emitting Diode (LED) and solar radiation. In one embodiment, said Cupriavidus necator bacteria comprises a H16_IPAl-10_int sequence as a chromosome integration.

In one embodiment, the present invention provides a composition comprising a live Cupriavidus necator bacteria strain comprising a CdS@ZnS (CZS) core-shell quantum dot, wherein said bacteria contains carbon dioxide molecules. In one embodiment, said bacteria contain said carbon dioxide molecules at a level greater than for said bacteria without a QD.

In one embodiment, the present invention provides a composition comprising a live Azobacteria vinelandii bacteria strain comprising a CdS@ZnS (CZS) core-shell quantum dot, wherein said bacteria contains carbon dioxide molecules.

In one embodiment, the present invention provides a method of carbon dioxide sequestration in a live bacteria strain, comprising, a) providing, i) a live bacteria comprising an enzyme that produces carbon dioxide (CO₂) and a quantum dot that transmits electrons having energies in the range of the reduction potential of said enzyme upon exposure to radiation for boosting production of said biofuel molecule, ii) an illumination source capable of emitting radiation, iii) a CO₂/O₂ 4:1 air mixture, iv) a fermentation solution, v) flushing said solution with said air mixture, and b) suspending said QD containing bacteria in said fermentation solution, c) flushing said fermentation solution containing QD containing bacteria with said air mixture, and d) irradiating said bacteria in said flushed fermentation solution under conditions such that said carbon dioxide is sequestered in said QD containing bacteria. In one embodiment, said flushing is at a rate of 0.5 standard liter per minute. In one embodiment, said flushing is for 15 minutes in duration before step d).

In one embodiment, the present invention provides a nanobiohybrid composition comprising a core-shell quantum dot (QD), wherein said core is selected from the group consisting of cadmium sulfide (CdS), cadmium selenide (CdSe), indium phosphide (InP), and copper zinc tin sulfide (Cu₂ZnSnS₄) (CZTS), wherein said shell is a two monolayer zinc sulfide (ZnS) shell, further comprising an enzyme. In one embodiment, said QD is attached to said enzyme. The composition further comprising a microorganism. In one embodiment, said microorganism is a bacterium. In one embodiment, said QD is inside of a microorganism, wherein said QD containing microorganism is a nanorg. In one embodiment, said enzyme is selected from the group consisting of a molybdenum-iron (MoFe) nitrogenase (MFN), MoFe-MFN subunits, Methyl Ketone pathway enzymes, 2,3-butanediol (BDO) pathway enzymes, isopropanol pathway enzymes, ethylene-forming enzyme (EFE), and PHB pathway enzymes. In one embodiment, said enzyme is molybdenum-iron (MoFe) nitrogenase (MFN). In one embodiment, said MFN is endogenous. The composition, wherein said MFN is heterologous. In one embodiment, said enzyme is MFN and said microorganism contains ammonia (NH₃) molecules. In one embodiment, said enzyme is MFN and said microorganism contains hydrogen (H2) molecules. In one embodiment, said molecules are at levels higher than for said microorganisms that does not contain a QD. In one embodiment, said enzyme is one or more enzyme in the Methyl Ketone (MK) pathway selected from the group consisting of enzymes encoded by genes: ‘tesA, fadB, Mlut_11700, and fadM. In one embodiment, said enzyme is one or more enzyme encoded by pJM20. In one embodiment, said Methyl Ketone (MK) pathway enzymes are endogenous. In one embodiment, said Methyl Ketone (MK) pathway enzymes are heterologous. In one embodiment, said microorganism is a bacterium. In one embodiment, said bacterium is C. necator strain H16. In one embodiment, said bacterium comprises pJM20. In one embodiment, said enzyme is encoded by pJM20 and said microorganism is a C. necator strain H16. In one embodiment, said enzymes form a complete MK pathway in said microorganism, wherein said microorganism contains C11-C15 methyl ketone compounds. In one embodiment, said C11-C15 methyl ketone compounds are at levels higher than for said microorganisms that does not contain a QD. In one embodiment, said enzyme is one or more enzyme in the isopropyl alcohol (IPA) forming pathway is selected from the group of enzyme encoding genes consisting of bktB(β-ketothiolase, H16_A1445) derived from C. necator H16; ctfAB(Succinyl-CoA transferase, AJ000086) from H. pylori, adc(acetoacetate decarboxylase, CA_P0165) from C. acetobutylicum, and sadh(secondary alcohol dehydrogenase, AAA23199.2) from C. beijerinckii. In one embodiment, said enzyme is one or more enzyme encoded in the H16_IPA1-10_int operon. In one embodiment, said d microorganism further comprises a H16_IPA1-10_int operon. In one embodiment, said microorganism has a chromosome, and said chromosome contains said H16_IPA1-10_int operon. In one embodiment, said microorganism contains IPA molecules. In one embodiment, said IPA molecules are at levels higher than for said microorganisms that does not contain a QD. In one embodiment, said enzyme is one or more enzyme in the 2,3-butanediol (BDO) forming pathway selected from the group of enzyme encoding genes consisting of alsS(acetolactate synsthase, BSU36010) and alsD(acetolactate decarboxylase, BSU36000) from B. subtilis and sadh(secondary alcohol dehydrogenase, AAA23199.2) from C. beijerinckii. In one embodiment, said enzyme is one or more enzyme encoded in the H16_BO2-20_int operon. In one embodiment, said microorganism further comprises a H16_BO2-20_int operon. In one embodiment, said microorganism has a chromosome, and said chromosome contains said H16_BO2-20_int operon. In one embodiment, said microorganism is a C. necator H16 strain. In one embodiment, said enzymes form a complete 2,3-butanediol pathway in said microorganism, wherein said microorganism is a bacterium contains BDO molecules. In one embodiment, said BDO molecules are at levels higher than for said microorganisms that does not contain a QD. In one embodiment, said enzyme is an ethylene-forming enzyme (EFE). In one embodiment, said EFE is derived from a Pseudomonas syringae bacterium. In one embodiment, said enzyme is an ethylene-forming enzyme (EFE). In one embodiment, said microorganism is a bacterium that contains ethylene molecules. In one embodiment, said ethylene molecules are at levels higher than for said microorganisms that does not contain a QD. In one embodiment, said enzyme is a Polyhydroxybutyrate (PHB) pathway enzyme. In one embodiment, said enzyme is a Polyhydroxybutyrate (PHB) pathway enzyme selected from the group consisting of PhaA, PhaB, and the heterodimeric PHB synthase PhaEC. The composition, wherein said enzyme is encoded by a PHB operon (phaCAB). In one embodiment, said enzyme is encoded by pBBR1-yfp. In one embodiment, said enzyme is a Polyhydroxybutyrate (PHB) pathway enzyme, wherein said microorganism is a bacterium contains PHB molecules. In one embodiment, said enzyme is a Polyhydroxybutyrate (PHB) pathway enzyme, wherein said microorganism is a bacterium contains CO₂ molecules. In one embodiment, said PHB molecules are at levels higher than for said microorganisms that does not contain a QD. In one embodiment, said CO₂ molecules are at levels higher than for said microorganisms that does not contain a QD. In one embodiment, said enzyme is a formic acid (FA) pathway enzyme. In one embodiment, said enzyme is a formate dehydrogenase. The composition, wherein said enzyme is a formate dehydrogenase derived from Clostridium carboxidivorans. In one embodiment, said bacterium contains formic acid molecules. In one embodiment, said enzyme is a methanol pathway enzyme. In one embodiment, said bacterium is lacking a functional formic acid (FA) pathway enzyme. In one embodiment, said bacterium contains hydrogen (H₂) molecules. In one embodiment, said bacterium contains methanol molecules. In one embodiment, said bacterium contains accumulated CO₂. In one embodiment, said formic acid molecules are at levels higher than for said microorganisms that does not contain a QD. In one embodiment, said hydrogen (H₂) molecules are at levels higher than for said microorganisms that does not contain a QD. In one embodiment, said methanol molecules are at levels higher than for said microorganisms that does not contain a QD. In one embodiment, said QD core-shell has a zwitterion L-cysteine (CYS) ligand cap. In one embodiment, said QD core has a ligand cap selected from the group consisting of a 3-mercaptopropionic acid (MPA) capping ligand and a cysteamine (CA) ligand cap. In one embodiment, said QD is selected from the group consisting of CZS1, CZS2, CZSe1, CZSe2, CZSe3, IPZS and CZTS. In one embodiment, said QD has an excitation energy from wavelengths ranging from ultraviolet to near-infrared energies.

In one embodiment, the present invention provides a method of using a nanobiohybrid comprising, providing, i) a core-shell quantum dot (QD), wherein said core is selected from the group consisting of cadmium sulfide (CdS), cadmium selenide (CdSe), indium phosphide (InP), and copper zinc tin sulfide (Cu₂ZnSnS₄) (CZTS), wherein said shell is a two monolayer zinc sulfide (ZnS) shell, ii) an enzyme for producing a product compound from a substrate, having a level of catalytic activity for said substrate, iii) a substrate for said enzyme, and iv) an illumination source capable of emitting radiation, and b) contacting said QD with said enzyme forming a nanobiohybrid, c) irradiating said nanobiohybrids in the presence of said substrate for emitting radiation which increases said catalytic activity of said enzyme for increasing production of a product compound. In one embodiment, said production of a product is greater than for said nanobiohybrid that is not irradiated. In one embodiment, said production of a product is greater than for said enzyme that is not contacting a QD. In one embodiment, said production ranges from 10⁵-10⁸ moles per mole of bacteria cells. In one embodiment, said nanohybrid is contained within a microorganism forming a nanorg. In one embodiment, the method further provides a fermentation media, and a step of contacting said nanorg with said fermentation media. In one embodiment, the method further comprises compounds selected from the group comprising CO₂, H₂O, O₂ and N₂. In one embodiment, said CdS QD absorbs radiation in the ultraviolet range. In one embodiment, said CdSe QDs absorbs radiation in the visible light range. In one embodiment, said InP and CZTS QDs absorbs radiation in the near-infrared (NIR) range. In one embodiment, said product compound is selected from the group consisting of ammonia (NH₃) molecules; hydrogen (H2) molecules; Methyl Ketones (MK) compounds; C11-C15 methyl ketone compounds; isopropyl alcohol (IPA); 2,3-butanediol (BDO); ethylene molecules; Polyhydroxybutyrate (PHB) molecules; CO₂ molecules; formic acid (FA) molecules; and methanol molecules. In one embodiment, said PHB is degradable.

In one embodiment, the present invention provides a composition comprising a metal nanoparticle and a molybdenum-iron nitrogenase (MFN/Mo—Fe) enzyme. In one embodiment, said metal nanoparticle is capped with a plurality of glutathione (GSH) capping ligands. In one embodiment, said MFN enzyme comprises a plurality of histidine tags. In one embodiment, said metal nanoparticle contacts said MFN enzyme. In one embodiment, said metal nanoparticle is attached to said histidine tags. In one embodiment, said metal nanoparticle is a gold (Au) nanoparticle. In one embodiment, said metal nanoparticle is a nanocluster (NC). In one embodiment, said metal nanocluster comprises a plurality of gold (Au) nanoparticles. In one embodiment, said gold nanocluster is selected from the group consisting of gold nanoparticles having 10-12 atoms (Au₁₀₋₁₂), 15 atoms (Au₁₅), 18 atoms (Au₁₈), 22 atoms (Au₂₂) and 25 atoms (Au₂₅). In one embodiment, said metal nanoparticle has an excitation energy from wavelengths ranging from ultraviolet to near-infrared energies. In one embodiment, said metal nanoparticle has a range of emission energies after exposure to said excitation energies. In one embodiment, said bacterium expresses said molybdenum-iron nitrogenase (Mo—Fe) enzyme having a reduction potential energy range matching said metal nanoparticle emission energies. In one embodiment, said metal nanoparticle further comprises a bacterium.

In one embodiment, the present invention provides a composition comprising an Azobacteria vinelandii bacteria strain comprising a gold nanoparticle, and a molybdenum-iron nitrogenase (Mo—Fe) enzyme. In one embodiment, said gold nanoparticle is a nanocluster (NC). In one embodiment, said method further comprising ammonia (NH₃) molecules. In one embodiment, said bacteria contains ammonia (NH₃) molecules above natural levels. In one embodiment, said bacteria contains hydrogen (H₂) molecules above natural levels. In one embodiment, said Mo—Fe enzyme is heterologous. In one embodiment, said Mo—Fe enzyme comprises a 7× histidine tag. In one embodiment, said bacteria comprise a cell lysate. In one embodiment, said bacteria are live bacteria. In one embodiment, said live bacteria are replicating. In one embodiment, said strain is DJ995. In one embodiment, said method further comprising CO₂/O₂ at a ratio of 4:1. In one embodiment, said composition further comprises a growth medium. In one embodiment, said growth medium comprises an accumulation of said NH₃ above levels for said bacteria cells without a metal nanoparticle.

In one embodiment, the present invention provides a method of producing ammonia (NH₃), comprising, a) providing, i) an A. vinelandii bacteria strain comprising a molybdenum-iron nitrogenase (MFN) enzyme, wherein expression of said MFN enzyme results in ammonia (NH₃) formation, a plurality of gold nanoparticles, ii) an illumination source capable of emitting radiation, iii) at least one compound selected from the group consisting of CO₂, H₂O, O₂ and N₂, and b) incubating said engineered bacteria in the presence of said at least one compound in the dark, and c) irradiating said engineered bacteria with said illumination source under conditions that produce ammonia (NH₃). In one embodiment, said gold nanoparticle is a nanocluster (NC). In one embodiment, said gold nanocluster is selected from the group consisting of gold nanoparticles having 22 atoms (Au₂₂) and 25 atoms (Au₂₅). In one embodiment, said production of NH₃ molecules is above natural levels. In one embodiment, said production of NH₃ is an amount greater than in said bacteria strain without said plurality of gold nanoparticles. In one embodiment, said production of NH₃ is an amount greater than in said bacteria strain without said irradiating. In one embodiment, said ammonia (NH₃) molecules are greater than 10⁵ moles of NH₃ per mole of bacteria cells. In one embodiment, said ammonia (NH₃) molecules are greater than 10⁵ up; to 10⁸ moles of NH₃ per mole of bacteria cells. In one embodiment, said hydrogen (H₂) molecules are greater than 10⁵ moles of NH₃ per mole of bacteria cells. In one embodiment, said Azobacteria vinelandii strain is DJ995. In one embodiment, said MFN enzyme is heterologous. In one embodiment, said method further providing a bacterial growth medium. In one embodiment, said bacteria are irradiated in said growth medium. In one embodiment, said NH₃ formation is in said growth medium. In one embodiment, said irradiation results in a yield amount greater than 10⁵ moles of NH₃ per mole of bacteria cells. In one embodiment, said bacteria comprise a cell lysate. In one embodiment, said bacteria are live bacteria. In one embodiment, said live bacteria are replicating. In one embodiment, said radiation source emits wavelengths selected from the group consisting of near-UV, visible, NIR radiation, Light Emitting Diode (LED) and solar radiation. In one embodiment, said illumination source emits 405 nm radiation. In one embodiment, said illumination source emits yellow (near-infrared (NIR)) radiation. In one embodiment, said growth medium further comprises L-ascorbic acid. In one embodiment, said growth medium further comprises 4-(2-hydroxyethyl)-1-piperazineethanesulfonic acid (HEPES) buffer.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The term, “enzyme” refers to a protein that acts as a catalyst to bring about a specific biochemical reaction, e.g. converting a specific set of reactants (called substrates) into specific products. An enzyme may also regulate the rate at which chemical reactions proceed in living organisms.

The term, “reduction potential” or “redox potential” or “oxidation reduction potential” refers to a tendency to gain electrons, i.e. become “reduced.

The term, “fuel” refers to a substance, such as a material or chemical, that is used to provide heat or power, usually by being burned. A fuel may be used as a single material or chemical and also as part of a fuel mixture comprising more than one material and/or chemical.

The term, “biofuel” refers to a fuel derived directly from living matter.

The term, “biodiesel” refers to a fuel comprising long-chain alkyl (methyl, ethyl, or propyl) esters, for example.

The term, “alcohol” refers to a compound in which one or more hydrogen atom positions in an alkane has a hydroxyl (—OH) group instead, including but not limited to primary (1°) alcohols where the C—OH is attached to one other carbon (on the end), e.g. Methanol CH₃OH (Methyl alcohol), ethanol CH₃CH₂OH (Ethyl alcohol), 1-Propanol CH₃CH₂CH₂OH (Propyl alcohol), 1-Butanol CH₃CH₂CH₂CH₂OH (Butyl alcohol), etc.; secondary (2°) alcohols where the C—OH is attached to two other carbons, e.g. 2-Propanol (CH₃)₂CHOH (IPA, Isopropanol, Isopropyl alcohol); and tertiary (3°) alcohols where the C—OH is attached to three other carbons (saturated carbon atom), e.g. 2-methylpropan-2-ol C₇H₁₆O₂, 2-methylbutan-2-ol CH₃CH₂C(CH₃)₂OH (tert-Amyl alcohol), butane diol, gasoline additives, gasoline substitutes, etc.

The term, “sequestration” refers to storage. As one example, sequestration of carbon refers to storage of carbon dioxide or other forms of carbon atoms within a microbe.

The term, “light” refers to electromagnetic radiation in a wavelength range including but not limited to infrared, visible, ultraviolet, etc.

The term, “illumination” refers to an action of supplying or brightening with light.

The term, “source” in reference to light and illumination refers to a natural light, e.g. sunlight; an artificial light source, such as fluorescent light, Light Emitting Diodes (LEDs), Surface Mounted Device LEDs, Chip on Board LEDs, etc.

An “illumination source” may also be referred to as a measurement of the total quantity of light emitted by the source expressed in lumens per unit of area.

The term, “purified” or “isolated”, as used herein, may refer to a peptide composition that has been subjected to treatment (i.e., for example, fractionation) to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to “apparent homogeneity” such that there is single protein species (i.e., for example, based upon SDS-PAGE or HPLC analysis). A purified composition is not intended to mean that all trace impurities have been removed.

As used herein, the term “substantially purified” refers to molecules, either nucleic or amino acid sequences, that are removed from their natural environment, isolated or separated, and are at least 60% free, preferably 75% free, and more preferably 90% free from other components with which they are naturally associated. An “isolated polynucleotide” is therefore a substantially purified polynucleotide.

A “variant” of an oligonucleotide or protein is defined as a nucleotide or amino acid sequence that differs from a wild type oligonucleotide or protein by having deletions, insertions and substitutions. These may be detected using a variety of methods (e.g., sequencing, hybridization assays etc.).

A “deletion” is defined as a change in either nucleotide or amino acid sequence in which one or more nucleotides or amino acid residues, respectively, are absent.

An “insertion” or “addition” is that change in a nucleotide or amino acid sequence which has resulted in the addition of one or more nucleotides or amino acid residues, respectively, as compared to, for example, a naturally occurring C. necator.

A “substitution” results from the replacement of one or more nucleotides or amino acids by different nucleotides or amino acids, respectively.

The term “derivative” as used herein, refers to any chemical modification of a nucleic acid or an amino acid. Illustrative of such modifications would be replacement of hydrogen by an alkyl, acyl, or amino group. For example, a nucleic acid derivative would encode a polypeptide which retains desired biological characteristics.

The term “transfection” or “transfected” refers to the introduction of foreign DNA into a cell.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “structural gene” refers to a DNA sequence coding for RNA or a protein. In contrast, “regulatory genes” are structural genes that encode products which control the expression of other genes (e.g., transcription factors).

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-C is an exemplary illustration showing designing artificial QD-cell nanorgs with a desired functionality.

FIG. 1A shows exemplary embodiments of contemplated and designed QD-bacteria nanorgs for light-induced air-H₂O—CO₂ reduction for targeted chemical generation, showing QD uptake, affinity binding and enzyme coupling, and light-triggered redox reaction. As illustrated here, QDs absorb sunlight in non-photosynthetic bacteria for direct solar-to-chemical fuel production using air and water as chemical feedstocks. FIG. 1B shows conduction/valence band (CB/VB) alignment of different semiconductor QDs, with labeled water (H⁺/H₂, O₂/H₂O), L-ascorbic acid (HA*/HA⁻), and dinitrogen (N₂/NH₄⁺) redox potential. FIG. 1C shows an exemplary emission image of different semiconductor QDs under 365 nm UV light excitation. (CZS: CdS@ZnS, CZSe: CdSe@ZnS, IPZS: InP@ZnS, CZTS: Cu₂ZnSnS₄@ZnS).

FIG. 2A-G is an exemplary illustration showing QDs designed for chemical coupling, energetic coupling between optoelectronic states and cellular process, appropriate interfaces, uptake and self-assembly, and ensuring biocompatibility. FIG. 2A shows biocatalytic product generation with QDs-CL enzyme biohybrids. The term “core” refers to QDs (CdS and CdSe) without ZnS shell, and the term “core/shell” refers to the QDs with a nominal 2ML ZnS shell. FIG. 2B shows net production of the cell lysate-QD hybrids, showing the light-activated H₂ and NH₃ generation using CZS QDs with variation of ZnS monolayers (0-3). FIG. 2C shows evolution of circuit (FIG. 13H) fit from the electrochemical impedance spectroscopy (EIS) data (FIG. 13G).

FIG. 2D shows cell viability (using resazurin assay) with cells treated with different ligand-capped QDs, calculated from FIGS. 13D-F. FIG. 2E shows inhibition of cell growth (using cell growth curve in nitrogen-free Burk media) with different ligand-capped QDs, calculated from FIGS. 20A-C. FIG. 2F shows cellular uptake of different ligand-capped QDs. FIG. 2G shows colony-forming unit of cells treated with different ligand-capped QDs. ASC5 refers to no quantum dots control treatment.

FIGS. 2AA-AE illustrates exemplary nanohybrids, i.e. nanorgs, and demonstrates exemplary affinity binding and self-assembly to His-tagged MFN in cell lysate using ZnS-coated QDs. FIGS. 17B-D is an exemplary illustration showing proof of selective binding of His-tagged MFN to the ZnS. Proof of selective binding of the His-tagged MFN to the ZnS.

FIG. 2AA shows exemplary schematic illustrations of the formation of QDs—A. vinelandii nanorgs. Left: Nanorgs generated with CdS@ZnS core-shell QDs, showing site-selective binding of His-tagged MFN to the zinc-rich facets of the QDs. Right: Nanorgs generated with CdS QDs, showing non-selective QDs to all cellular components binding.

FIG. 2AB demonstrates exemplary SDS-PAGE of protein samples. Lane 1 and 6: protein molecular weight marker (From top to bottom: 116.0, 66.2, 45.0, 35.0, 25.0, 18.4, 14.4 kDa). Lane 2: cell lysate from A. vinelandii DJ995. Lane 3: purified MoFe nitrogenase (elution from Zn-IMAC column). Lane 4: protein bound to CdS. Lane 5: protein bound to ZnS.

FIG. 2AC demonstrates exemplary agarose gel electrophoresis of QDs-cell lysate (a, b) and QDs-nitrogenase mixtures (FIG. 2Ac, FIG. 2Ad). The migration of QDs and the proteins were indicated by fluorescence (FIG. 2Aa, FIG. 2Ac) and Coomassie blue staining (FIG. 2Ab, FIG. 2Ad). Lane 1: cell lysate; Lane 2: CZSe1-cell lysate mixture; Lane 3: CZSe2-cell lysate mixture; Lane 4: purified MFN; Lane 5: CZSe1-purified MFN; Lane 6: CZSe2-purified MFN; Lane 7: CZSe1; Lane 8: CZSe2.

FIG. 2AD demonstrates exemplary Cadmium detected from the MFN and non-MFN band of the CZSe2-cell lysate AGE lane (FIG. 2AB, lane 3). The residual cadmium detected in non-MFN band was limited mainly by the detection limit of the instrument.

FIG. 2AE demonstrates exemplary biocatalytic product generation (hydrogen production, converted to total electron generation) with QDs-CL enzyme biohybrids. The term “core” refers to QDs (CdS and CdSe) without ZnS shell, and the term “core/shell” refers to the QDs with nominal 2ML ZnS shell.

FIG. 3A-F shows exemplary results during the designing QDs for chemical coupling, energetic coupling between optoelectronic states and cellular process, appropriate interfaces, uptake and self-assembly, and ensuring biocompatibility. FIG. 3A shows exemplary Net production of the cell lysate-QD hybrids, showing the light-activated H₂ and NH₃ generation using CZS QDs with variation of ZnS monolayers (0˜3). FIG. 3B shows exemplary Evolution of Cₜₒₜₐₗ (C_{sc}+Cₜ) and R_{sc} with the increase of ZnS shell thickness, obtained from the equivalent circuit (FIG. 13H) fit from the electrochemical impedance spectroscopy (EIS) data (FIG. 13G).

FIG. 3C shows exemplary Inhibition of cell growth (using cell growth curve in nitrogen-free Burk media, under dark condition) with different ligand-capped QDs, calculated from FIG. S8a-c.

FIG. 3D shows exemplary Cell viability (using resazurin assay) with cells treated with different ligand-capped QDs (after performing the photocatalytic tests), calculated from FIG. S9d-f. FIG. 3E shows exemplary Colony forming unit of cells treated with different ligand-capped QDs. “Control” refers to no quantum dots control treatment. FIG. 3F shows exemplary Cellular uptake of different ligand-capped QDs.

FIG. 4A-F is an exemplary illustration showing evidence of light-driven chemical generation from nanorgs.

FIG. 4A shows different bacteria strains for NH₃ (A. vinelandii DJ995 and DJ1003) and C₂H₄(Ethylene) (C. necator pBBRl-efe and pBBRl-YFP) production.

FIG. 4B shows exemplary ammonia production from nanorgs made from A. vinelandii with His-tagged MFN (DJ995, indicated as “H”) or non-His-tagged MFN (wild-type, indicated as “NH”) and CdS (without ZnS shell) or CZS (with two-monolayer ZnS shell) QDs.

FIG. 4C shows exemplary production of C₂H₄ and NH₃ in CO₂/air or argon.

FIG. 4D shows exemplary NH₃ and C₂H₄ production with (0.5 mM) and without the addition of ionophore (2,4-DNP).

FIG. 4E shows exemplary production of C₂H₄ and NH₃ in CO₂/air or argon.

FIG. 4F shows NH₃ and C₂H₄ production with and without the addition of sacrificial donor/quencher (5 mM L-ascorbic acid).

FIG. 5A-G is an exemplary illustration showing nanorg microbial factories for (i.e. producing) fuel and chemical production with variations of different QDs, bacterial strains, and light photon energies and flux.

FIG. 5A shows NH₃ turnover number (TON) with nanorgs made from different QDs.

FIG. 5B shows turnover frequency (TOF) of different fuels with nanorgs made from CZS QDs and different exemplary bacteria strains.

FIG. 5C shows cumulative specific productivity of different fuels and biochemical (represented as per gram cell dry weight per day) with nanorgs made from CZS QDs and different bacteria strains.

FIGS. 5D-E shows time traces, represented as against absorbed photon numbers, of NH₃, H₂ (with CZS QDs and A. vinelandii DJ955), and C₂H₄ (with CZS or IPZS QDs and C. necator pBBRl-efe) production, showing the turnover number and quantum yield with time.

FIG. 5F shows C₂H₄ production (with IPZS QDs and C. necator pBBRl-efe) under different light sources for irradiation.

FIG. 5G shows exemplary comparison of chemical production yield for PHB, C₂H₄, MKS, IPA, and BDO, e.g. as biofuel production, using light-activated nanorgs and the natural growth (organolithotrophic with sugars) conditions. Strains were generated as detailed herein. The exemplary 3D plot represents nanorgs-to-natural production ratios.

FIG. 6 is an exemplary illustration showing one embodiment of a scaled-up production using nanorgs in a bioreactor. (Upper Left) A photobioreactor with 4 liters of nanorgs-buffered water suspension, with wild-type C. necator comprising pBBRl-YFP, CZS2 QDs, 400 nm light irradiation, for PHB production. (Upper Right) The PHB extracted from the nanorgs, showing an example of a total dry weight of 1.0 gram. Lower panels show another exemplary illustration showing scaled-up production of PHB, showing the process of nanorgs fermentation in one example of a photobioreactor, digest and PHB extraction with sodium hypochlorite/chloroform mixture, PHB precipitation with methanol/water from chloroform extract, coagulation of PHB, PHB drying (showing the dry weight of PHB), and casted PHB thin film from glacial acetic acid solution.

FIG. 7 is an exemplary illustration showing Scheme S1. The CdS@ZnS core-shell QDs, where Di (3.55 nm) and D2 are the diameter of the CdS core and the whole CdS@ZnS QDs, respectively.

FIG. 8 is an exemplary illustration showing constructs used in IPA and 2,3-BDO strains and integrated into the genome of C. necator.

FIG. 9A: Band positions of CdS and CdSe nanoparticles (black: CdSl or CdSel, red: CdS2 or CdSe2, blue: CdS3 or CdSe3) showing their bandgap, conduction band (CB) and valence band (VB) position (vs. NHE at pH=7). Water redox potentials are also labeled,

FIG. 9B: Net light-induced hydrogen production from the quantum dot-MoFe nitrogenase biohybrids, in an argon atmosphere,

FIG. 9C: Net light-induced hydrogen production from the quantum dot-cell lysate mixture, in an argon atmosphere.

FIG. 9D: Net light-induced hydrogen and ammonia production from the quantum dot-cell lysate mixture, in dinitrogen atmosphere. 0ML-3ML refer to CdS@ZnS core-shell nanoparticles with 0˜3 monolayer ZnS shells, respectively.

FIG. 10A: Cell growth inhibition in Burk media with nanoparticles at 200 nM, calculated from FIG. 29.

FIG. 10B: The viability of cells treated with nanoparticle-containing ASC5 media (35 mM FfEPES, 5 mM L-ascorbic acid, pH=7.4), calculated from the resazurin assay result shown in FIG. 35

FIG. 10C: Colony forming unit and the calculated cell viability (no nanoparticle treatment as 100%) of cells treated in ASC5 media with nanoparticles at 500 nM. The initial OD₆oo is 10″⁴. MPA (CZS-MPA), CYS (CZS-CYS), and CA (CZS-CA) refer to 3-mercaptopropionic acid, L-cysteine, and cysteamine-coated CdS@ZnS2ML nanoparticles. CdS-CYS refers to cysteine-coated CdS nanoparticles.

FIG. 11A & FIG. 11B: Photocatalytic ammonia turnover number (TON) in the air and pure dinitrogen atmosphere (at t=1 h). The reaction phase contains (FIG. 11a) 200 nM or (b) 500 nM nanoparticles and OD₆oo⁼LO bacteria cells. MPA, CYS, and CA refer to CdS@ZnS2ML nanoparticles with 3-mercaptopropionic acid, L-cysteine, and cysteamine capping ligand.

FIG. 11C: Photocatalytic hydrogen and ammonia TON in ASC5, ASC10, ASC25 media (with L-ascorbic acid at 5, 10, 25 nM, respectively). The reaction phase contains 500 nM CYS-coated nanoparticles and OD6oo⁼l-0 bacteria cells,

FIG. 11D: Photocatalytic hydrogen and ammonia TON of ASC5-CYS500 (CYS-coated nanoparticles at 500 nM, bacteria cell at OD₆₀₀=1.0 in ASC5 media).

FIGS. 12A-F are exemplary illustrations showing UV-VIS spectra and photoluminescence (PL) spectra of the nanoparticles with different sizes for use in QDs of the present inventions.

FIG. 12A shows CdS (three sizes: 3.6, 4.2, 5.1 nm from 1 to 3) UV-VIS spectra;

FIG. 12B shows CdSe (three sizes: 2.3, 2.6, 4.6 nm from 1 to 3) UV-VIS spectra;

FIG. 12C shows CZSI (CdS@ZnS core-shell with nominal 0-3 monolayer ZnS shell) UV-VIS spectra;

FIG. 12D shows CZSI PL spectra. QDs UV-VIS.

FIG. 12E and PL Fig. shows FIG. 12F spectra. In FIG. 12E and FIG. 12F, shows QDs having a nominal 2 monolayer ZnS shell.

FIG. 13 presents OCP changes upon light “turn-on” (decrease of OCP, as shown in the vertical line) and “turn-off” (an exponential increase of OCP). FIG. 13A-D Electrochemical impedance spectroscopy (EIS) of (FIG. 13J, E) CdS and (FIG. 13K, (FIG. 13F) CdSe nanoparticle electrodes, represented as Nyquist plots, (FIG. 13E, FIG. 13F) shows the high-frequency part (lower impedance) of the spectra in (FIG. 13J, FIG. 13K). FIG. 13H Equivalent circuit used to fit the EIS spectra of CdS@ZnS nanoparticle electrode. This equivalent circuit contains three parts for electrolyte (Rₛ: solution resistance), interface (Rcₜ: charge transfer resistance, Cai: double-layer capacitance) and semiconductor bulk (C_{sc}, Rs_{C}: space charge layer capacitance and resistance; Cₜ, Rₜ: capacitance and resistance from defect states). Based on this equivalent circuit, charge trapping in the nanoparticles and charge tunneling through the ZnS shell could be evaluated from the Ctotal (Csc+Cₜ) and R_{sc}, respectively, (FIG. 13L), (FIG. 13G) EIS spectra of CdS@ZnS nanoparticle electrode ((FIG. 13G is the same as Fig. FIG. 13L with a smaller scale for better showing the spectra of 0-2 ML samples), presented as the Nyquist plot, (FIG. 13M) Evolution of Ctotal (C_{sc}+Cₜ) and Rs_{C} with ZnS layer increase. Significant (by 1 magnitude) decrease of Ctotal was observed in samples with 2 and 3 ML ZnS shell compared to 0 or 1 ML samples, indicating notably reduced charge trapping and hence decreased charge recombination. R_{sc} slightly increases with the addition of ZnS shells but still remains small for 2 ML sample. A remarkable increase of R_{sc} was observed when the third ZnS shell was deposited, indicating a significant blocking effect for charge tunneling to the nanoparticle surface.

FIGS. 13A-M are exemplary illustrations showing electrochemical characterization of the QDs. Measured by Electrochemical impedance spectroscopy (EIS).

FIGS. 13A-D Differential pulse voltammetry of FIG. 13A-B CdS and FIG. 13C-D CdSe QDs colloidal suspension, showing the voltammogram for FIG. 13A, FIG. 13C, backward and (FIG. 13b, FIG. 13d) forward scan. The arrows indicate their FIG. 13A, 13C, CB and FIG. 13B, 13D, VB positions (vs. NHE).

FIGS. 13E-FIG. 13F shows EIS (Nyquist plots) of FIG. 13E CdS and FIG. 13F, shows CdSe QD electrodes.

FIG. 13G shows EIS (Nyquist plots) of CZSI QD (with nominal 0-3 monolayer ZnS shell) electrode.

FIG. 13H shows equivalent circuit used to fit the EIS spectra of CdS@ZnS QD electrodes (29, 30). This equivalent circuit contains three parts for the electrolyte (Rₛ: solution resistance), interface (R_{c}t: charge transfer resistance, Cdi: double-layer capacitance) and semiconductor bulk (C_{sc}, R_{sc}: capacitance and resistance of space charge layer; Cₜ, Rₜ: capacitance and resistance of defect states). Charge trapping in the QDs and charge tunneling through the ZnS shell could be evaluated from the Ctotai (C_{sc}+Ct) and Rsc, respectively.

FIG. 13I OCP changes upon light “turn-on” (a fast decrease in OCP, as shown in the vertical line) and “turn-off (an exponential increase in OCP).

FIG. 14 is an exemplary illustration showing Zeta potential CYS-capped CZS. Zeta potential CYS-capped CZS, suspended in water with different pH values.

FIG. 15A-E is an exemplary illustration showing Natural growth and production with different C. necator strains, showing accumulation of products over time (PHB, C₂H₄, IPA, BDO, and MK) in the growth media.

FIG. 16A-H is an exemplary illustration showing proof of QDs and cellular protein binding. Photoluminescence and UV-VIS spectra of the CL and QDs-CL solutions after incubation FIG. 16A, FIG. 16B, the supernatants after centrifugation. FIG. 165C, FIG. 16D, and the redispersed solutions FIG. 16E, FIG. 16F. Inset photographs taken with a UV lamp show the condition of each solution prior to the recording of the spectra.

FIG. 16G and FIG. 16H shows exemplary ATR-FTIR of the QDs-cell lysate (QDs-CL), cell lysate (CL), and QDs. The cell lysate was prepared from Fig. FIG. 16G A. vinelandii DJ995 and Fig. FIG. 16H C. necator pBBRl-efe.

FIG. 17A-F shows exemplary light-induced H₂ or NH₃ production with QDs-MFN or QDs-CL biohybrids. In FIG. S7B-F, CZSI (with nominal 2ML ZnS shell) QDs were used.

FIG. 17A shows exemplary net H₂ TON with QDs-MFN biohybrids in light-induced water reduction (argon atmosphere). Net H₂ generation FIG. 17B in light-induced water reduction (argon atmosphere) and net H₂/NH₃ generation FIG. 17C in light-induced dinitrogen-water reduction (dinitrogen atmosphere) with QDs-CL biohybrids.

FIG. 17D-F shows exemplary light-induced H₂ generation with QDs-CL biohybrids, showing the control FIG. 17D and the effects of additional imidazole FIG. 17E or using lower pH media FIG. 17F.

FIG. 17G shows exemplary representative gas-chromatography showing H₂ production from light-driven water reduction (same as FIG. 17D).

FIG. 17H shows exemplary representative fluorescent assay showing NH₃ production from light-driven dinitrogen-water reduction (same as FIG. 17C).

FIGS. 18A-L show exemplary laser scanning confocal images. FIG. 18A-C shows laser scanning confocal images of a large cell cluster with internalized QDs.

FIG. 18D shows a laser scanning confocal images of a single cell with internalized QDs seen throughout its structure. FIG. 18E-L shows laser scanning confocal images of a single cell as the focus of sequential images changes from one side FIG. 18E to the other side of the cell FIG. 18L. FIG. 18E-J: 1.000 um, 1.162 um, 1.295 um, 1.502 um. 1.691 um, 1,744 um, respectively.

FIGS. 19A-L present exemplary results showing Cell growth curve assay. Cell growth curve assay. FIG. 19A-C shows the growth of A. vinelandii DJ995 in the nitrogen-free Burk media with MPA, CYS, CA-capped CZSI QDs at various concentration.

FIG. 19D shows the same test as FIG. 19A-C, with CYS-capped CdSl QDs (no ZnS shell).

FIGS. 19E-F show the growth of A. vinelandii DJ995 in the photocatalytic media (HEPES with supplied L-ascorbic acid at different concentration), without FIG. 19E or with FIG. 19F the presence of QDs (CYS-capped CZSI).

FIGS. 19G-I show the growth of A. vinelandii DJ995 in the nitrogen-free Burk media after treatment with MPA, CYS, CA-capped QDs-containing photocatalytic media (with 5 mM L-ascorbic acid) in the dark.

FIGS. 19J-L show the same tests as FIG. 19G-I, but with light irradiation (2 h) treatment.

FIG. 20A-F presents exemplary results showing cell viability tests with resazurin. Cell viability tests with resazurin. FIG. 20A-C shows cells treated with MPA, CYS, CA-capped CZSI QDs in photocatalytic media (supplied with 5 mM L-ascorbic acid) in the dark. FIG. 20D-F shows the same assay used for FIG. 20A-C with cell treatment under light irradiation.

FIGS. 21A-I present exemplary results showing Control experiments with A. vinelandii DJ995 and C. necator pBBRl-efe. Control experiments (NH₃ and C₂H₄ production) with A. vinelandii DJ995, FIG. 21A, FIG. 21C, FIG. 21E, and C. necator pBBRl-efe. FIG. 21B, FIG. 21E.

FIG. 21F. FIG. 21A-B shows production of NH₃ and ethylene with bacteria cells or QDs, the complete nanorgs (cells with QDs) were plotted as a comparison. FIG. 21C-D shows NH₃ and C₂H₄ production in the air, carbon dioxide atmosphere compared to the same test in argon. FIG. 21E-F shows NH₃ and C₂H₄ production with or without the presence of ionophore (2,4-DNP).

FIG. 21G shows exemplary gas chromatography showing the production of ethylene.

FIG. 21H shows exemplary mass spectra of the headspace gas from nanorg tests using CZS QDs with ethylene and IPA-producing strains, showing the detection of ¹³C₂H₄⁺ cation (m/q=30, NIST MS numbers 18814) from ethylene-producing strain. No such peak with IPA-producing strain also proves production of no ethylene production from other components (e.g. QDs) in the nanorg systems.

FIG. 21I shows exemplary mass spectra of the headspace gas showing the production of ¹³C-labelled isopropanol (m/q=47 fragment, NIST MS numbers 289584) from IPA-producing strain.

FIG. 22A-I is an exemplary illustration showing optimization of experimental parameters for improved NH₃ production. Optimization of experimental parameters for improved NH₃ production.

FIG. 22A shows NH₃ generation with cell optical density (with 200 nM MPA-capped CZSI QDs).

FIG. 22B shows NH₃ TON with MPA, CYS, and CA-capped QDs at different concentration. FIG. 22C-D shows NH₃ production with (FIG. 22C) 200 nM or (FIG. 22D) 500 nM CZS 1 QDs performed in air and pure nitrogen atmosphere. FIG. 22E-F shows NH₃ TON with irradiation intensity FIG. 22E at 1 hour and FIG. 22F with time under high (1.6 mW/cm²) and low (0.67 mW/cm²) photon flux.

FIG. 22G shows NH₃ TON in different media with or without a sacrificial agent (L-ascorbic acid, or sulfide). Media contain 35 mM electrolyte. ASC5: 5 mM L-ascorbic acid in 35 mM HEPES buffer, HEPES-S: 5 mM sodium sulfide in 35 mM HEPES buffer.

FIG. 22H shows Time evolution of NH₃ with and without L-ascorbic acid as a sacrificial donor.

FIG. 22I shows NH₃ TON with time in photocatalytic (ASC5, ASC10, ASC25 refers to 5, 10, and 25 mM L-ascorbic acid in 35 mM HEPES buffer) and Burk media.

FIG. 23 is an exemplary chart showing results of a recovery test for NH₃ production. It shows one embodiment, NH₃ production rate (1.5-hour time point) and the recovery of the NH₃ TON for three running cycles.

FIG. 24 is an exemplary illustration showing TON of different fuels. This 3D plot represents the TON of different fuels generated from light-activated (400 nm) nanorgs prepared by coupling A. vinelandii DJ995 and different genetically-engineered (section: C. necator Culture) C. necator strains with different QDs (section: CdS and CdSe QDs, InP@ZnS core-shell QDs (IPZS)).

FIG. 25 shows a calibration curve used, for example, in a fluorescence ammonia assay.

FIGS. 26A-B demonstrate exemplary data showing light-induced hydrogen production using (FIG. 26A) CdS and (FIG. 26B) CdSe nanoparticles with or without coupling to MFN.

FIGS. 27A-C show exemplary data of photocatalytic proton reduction using CdS@ZnS nanoparticle-cell lysate biohybrids under 400 nm irradiation in (FIG. 27A) pH 7.4, 100 mM L-ascorbic acid, (FIG. 27B) pH 7.4, 100 mM L-ascorbic acid with 250 mM imidazole, and (FIG. 27C) pH 5.9, 100 mM L-ascorbic acid. NP and NP-CL refer to photocatalytic reaction with CdS@ZnS nanoparticles and nanoparticle-cell lysate biohybrids, respectively. xML (x=0 to approximately 3) refers to numbers of nominal ZnS coating.

FIGS. 28A-B show exemplary data of photocatalytic dinitrogen reduction using CdS@ZnS nanoparticle-cell lysate biohybrids under 400 nm light irradiation, and the generation of hydrogen and ammonia were presented in (28A) and (28B), respectively. NP and NP-CL refer to photocatalytic reaction with CdS@ZnS nanoparticles and nanoparticle-cell lysate biohybrids, respectively. xML (x=0˜3) refers to numbers of nominal ZnS coating.

FIG. 29 shows exemplary cell growth in nitrogen-free Burk media with different concentrations of nanoparticles. MPA, CYS, CA refer to nanoparticles with 3-mercaptopropionic acid, L-cysteine and cysteamine capping ligand, respectively. CZS and CdS refer to CdS@ZnS nanoparticles with 2ML and OML ZnS shell. Blank refers to cell growth in Burk media without nanoparticles. The numbers (50-1000) are the nanoparticle concentration.

FIG. 30 shows exemplary cell growth in nitrogen-free Burk media after treating the cells with photocatalytic media ASC5 (5 mM L-ascorbic acid, 35 mM HEPES, pH=7.4) or Burk media for 2 hours in dark. MPAx, CYSx, CAx (x=50, 100, 200, 500, 750, 1000 indicating the concentration of nanoparticles in nM) refer to cell treatments in ASC5 media containing nanoparticles with 3-mercaptopropionic acid, L-cysteine and cysteamine capping ligand, respectively (the same below). ASC5C and BMC refer to cell treatments in ASC5 media and Burk media without nanoparticles (the same below).

FIG. 31 shows exemplary cell growth in nitrogen-free Burk media after treating the cells with photocatalytic media ASC5 or Burk media for 2 hours with 400 nm irradiation at 1.6 m W/cm. The notations are the same as in FIG. 30.

FIG. 32 shows exemplary cell growth in ASC5 media in the dark (e.g., the absence of light). ASC5C, ASC10C, and ASC25C refer to cell growth in photocatalytic media (5, 10, 25 mM L-ascorbic acid, 35 mM HEPES, pH 7.4) without nanoparticles. ASC5-CYS, ASC10-CYS, and ASC25-CYS refer to cell growth in photocatalytic media with cysteine-coated nanoparticles at 500 nM. ASC5N refers to nanoparticles in ASC5 media (without cells).

FIG. 33 provides exemplary data of a resazurin assay for testing cell viability after nanoparticle treatment in photocatalytic media or Burk media for 2 hours in dark. The notations are the same as in FIG. 30 and FIG. 32. BM-CYS refers to cell treatment with 500 nM cysteine-coated nanoparticles in Burk media.

FIGS. 34A-C provide exemplary cell viability data (cell treatment in dark) calculated from the resazurin assay in FIG. 33, by taking the slope of the rising part of the time-dependent fluorescence curve and compared to the control, (FIG. 34a) The ASC5C (mentioned above, and as described herein) is used as control (cell viability=100%) and cell viability of MPAx, CYSx, CAx is presented, (FIG. 34b) Cell viability of cell treatment with 500 nM L-cysteine coated nanoparticles in different media, using each media (without nanoparticles) as a control, (FIG. 34c) Cell viability with cell treatment in different media (without nanoparticles), using cell treatment in Burk media as a control.

FIG. 35 presents exemplary data of a resazurin assay for testing cell viability after treating the cells with photocatalytic media or Burk media with different concentrations for 2 hours with 1.6 mW/cm² 400 nm light irradiation. The notations are the same as in FIG. 33.

FIG. 36 presents exemplary data of ammonia generation with varying Azotobacter vinelandii DJ995 cell optical density.

FIG. 37 presents exemplary data of ammonia turnover number (TON, mol NF\hat{ }/mol cells) with fixed cell optical density (OD6oo⁼l-0) and nanoparticles with varied capping ligands and concentration.

FIG. 38 presents exemplary data of control experiments with the removal of nanoparticles (Cells) or cells (MPA500, CYS500, and CA500) from the complete mixture. MPA500, CYS500, and CA500 refer to nanoparticles with correspondent capping ligands at 500 nM.

FIG. 39 presents exemplary data of ammonia TON with irradiation intensity.

FIG. 40A presents exemplary data of ammonia generation with time in photocatalytic and Burk media. The reaction phase (1 ml) contains 500 nM CYS-coated nanoparticles, OD6oo⁼LO bacteria cells. ASC5, ASC10, ASC25, and BM refer to photocatalytic media (35 mM HEPES with 5, 10, 25 mM L-ascorbic acid at pH 7.4) and Burk media, respectively.

FIG. 40B Photos of Azotobacter vinelandii DJ995 cell pellets (left) and cell lysate (right).

FIGS. 41A-B presents exemplary data of ammonia TON in ASC5 media with 500 nM CYS-coated nanoparticles and ODeoo\hat{ }l-O bacteria cells, (FIG. 41a) The photocatalytic reaction was continued for 3 cycles, each with 1.5 hours. At the end of each cycle, the cells were centrifuged down and recharged with new media, (FIG. 41b) Net ammonia production is presented at 1.5 hour time point in each cycle, and the recovery (with the first cycle as 100%) of the cell in generating ammonia was calculated.

FIGS. 42A-B presents exemplary data of photocatalytic hydrogen generation in ASC5, ASC10, ASC25 media. The reaction phase contains 500 nM CYS-coated nanoparticles and OD6oo-l-0 bacteria cells.

FIGS. 43A-E shows exemplary selection of different atomically-precise Au NCs for forming nanorgs. FIG. 43a Ultraviolet-visible (UV-VIS) spectra, FIG. 43b hydrodynamic size (measured by dynamic light scattering), FIG. 43c zeta potential, FIG. 43d redox potential (showing the conduction band, measured by differential pulse voltammetry) of different sized Au NCs. FIG. 43e Schematic diagram of electron transfer from light-activated Au NCs to nitrogenase for dinitrogen reduction to ammonia, with energy levels for different Au NCs conduction band and Mo—Fe nitrogenase reduction potential.

FIGS. 44A-D shows exemplary cellular uptake and biocompatibility of Au NCs. FIG. 44A Cellular uptake of different sized Au NCs by A. vinelandii. FIG. 44b A. vinelandii growth curve with different sized 20 μM Au NCs, incubated in dark and with light irradiation. FIG. 44c A 3D plot showing the percentage of growth (compared to cell growth with no Au NCs as 100%) under light irradiation, calculated from the corresponding growth curves at 24-hour time point. FIG. 44d Resazurin dye assay to evaluate the cell viability after 4 hour Au NCs treatment under light irradiation. Viability is calculated using treatment with no Au NCs as reference (100%).

FIGS. 45A-B shows exemplary influence of biocompatibility and chemical coupling between Au NCs-enzymes in nanorgs. Light-driven ammonia production (a) using nanorgs constructed from Au₁₈ or Au₂₂ NCs with A. vinelandii DJ995 strain, conducted in different atmospheres; and (b) using nanorgs constructed from Au₂₂ NCs with different A. vinelandii strains.

FIGS. 46A-D shows exemplary light-driven biocatalytic reaction efficiency using nanorgs from different Au NCs. FIG. 46A Time-dependent ammonia production with Au₂₂ NC-nanorgs. FIG. 46b A 3D plot showing the ammonia turnover number using nanorgs made from different Au NCs at different concentration. FIG. 46c Ammonia turnover frequency using nanorgs made from different Au NCs (8 μM). FIG. 46d Calculated quantum efficiency of each optimized nanorgs, with the theoretical maximum quantum efficiency limited by enzyme turnover.

FIGS. 47A-C shows exemplary biocompatibility tests of small Au NCs. Growth curves for (FIG. 47a) Au₁₀₋₁₂ and (FIG. 47b) Au₁₅, compared to cell growth with no Au NCs, under light irradiation. (FIG. 47c) cell viability (resazurin dye assay, compared to no Au NCs as 100%) after treatment under light irradiation for 4 hours.

FIG. 48 shows exemplary light-driven ammonia production using nanorgs with small Au NCs. Turnover number for the biocatalytic reaction of ammonia production from air using nanorgs formed from A. vinelandii DJ995 strain with Au₁₀₋₁₂, and Au₁₅ NCs. The variation of ammonia production with increasing Au NC concentration tracks well with an initial increase in light absorption, subsequent saturation of nano-biohybrids, and subsequent loss of cell viability (FIG. 47) at higher concentrations.

### DETAILED DESCRIPTION OF THE INVENTION

The invention relates to a nano-biohybrid (e.g., a nanoorg) organism (e.g. a bacteria) comprising different core-shell quantum dots (QDs) or gold nanoparticle clusters, with excitation ranges having ultraviolet to near-infrared energies, coupled with targeted enzyme sites. When illuminated by light, these nanorgs drive the renewable production of biofuel molecules and chemicals using carbon-dioxide (CO₂), water, and nitrogen (from air) as substrates. The disclosed nanorgs catalyze light-induced air-water-CO₂ reduction with a high turnover number (TON) of approximately 10⁶-10⁸ (mols of product per mol of cells) to biofuels including, but not limited to, isopropanol (IPA), butane diol, gasoline additives, gasoline substitutes, 2,3-butanediol (BDO), C11-C15 methyl ketones (MKs), and hydrogen (H2); and chemicals such as formic acid (FA), ammonia (NH₃), ethylene (C₂H₄), and degradable bioplastics, e.g. polyhydroxybutyrate (PHB). These nanorg cells function as nano-microbial factories powered by light.

In one embodiment, the present invention contemplates a nano-biohybrid organism (or nanorg) including, but not limited to: (1) chemical coupling via affinity binding and self-assembly, (2) the energetic coupling between optoelectronic states of artificial materials with a cellular process, and (3) the design of appropriate interfaces ensuring biocompatibility. Here it is shown that at least seven different core-shell quantum dots (QDs), with excitations ranging from ultraviolet to near-infrared energies, coupled with targeted enzyme sites in bacteria. When illuminated by the appropriate light energy, these QDs drive the renewable production of biofuels and chemicals using carbon-dioxide (CO₂), water, and nitrogen (e.g. from air) as substrates. These disclosed QDs, as described herein, use zinc-rich shell facets for affinity attachment to cellular proteins. Cysteine zwitterion ligands enable uptake through the cell wall, facilitating cell survival. Together, such compositions provide nanorgs catalyzing light-induced air-water-CO₂ reduction with a high turnover number (TON) of approximately 10⁶-10⁸ (mols of product per mol of cells) to biofuels, such as isopropanol (IPA), 2,3-butanediol (BDO), C₁₁-C₁₅ methyl ketones (MKs), and hydrogen (H₂); and chemicals such as formic acid (FA), ammonia (NH₃), ethylene (C₂H₄), and degradable bioplastics such as polyhydroxybutyrate (PHB). Therefore, these resting cells function as nano-microbial factories powered by light.

External control (e.g. wireless) over specific cellular function has been a long-standing objective in biology (Ref. 1). While such control, e.g. external regulation, can provide unprecedented insights into molecular biology, it can also form the basis for several new biotechnological techniques ranging from diagnosis and therapeutics to the generation of biofuels and bioproducts.

In one embodiment, the present invention contemplates a platform technology comprising nano-biohybrid organisms (or nanorgs) using semiconductor nanoparticles which can be designed for affinity binding to desired proteins by facile transport, uptake, and self-assembly, and matched to the electrochemical potential of the enzyme to trigger them externally using electromagnetic radiation, e.g. light, etc. As a specific application and to demonstrate broader applicability of methods described herein, the formation of such living nano-biohybrid organisms (or nanorgs) using nonlimiting strains of Azotobacter vinelandii and Cupriavidus necator bacteria strains is shown with a desired enzyme activation for targeted chemical generation using light in these non-photosynthetic microbes.

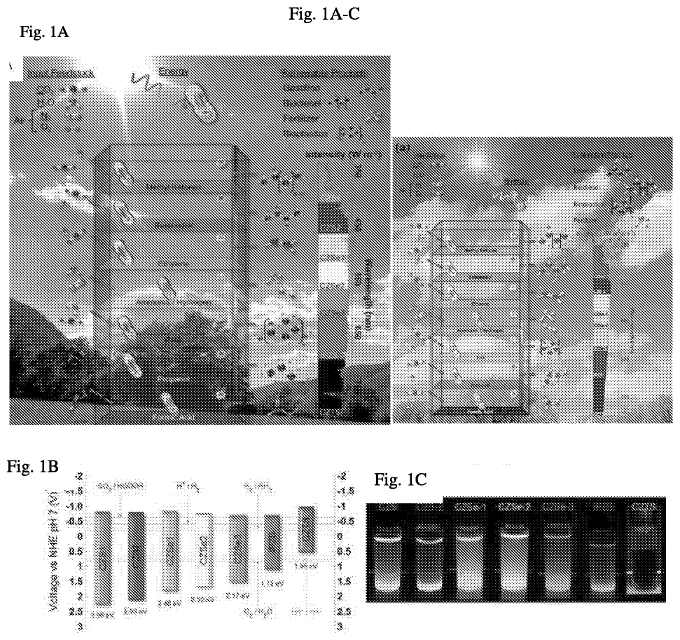

In one embodiment, the presently contemplated engineered strains of naturally occurring and synthetic bacteria can accomplish industrial reactions using chemical energy to generate electrons and reduce renewable chemical feedstocks like, for e.g., CO₂, H₂O, and air, and can be labeled as living factories (FIG. 1A) (Ref. 2). Both A. vinelandii and C. necator normally derive the energy needed for the chemical transformations of feedstocks to biofuels from sugars, since such non-photosynthetic microbes cannot directly utilize the sunlight like, for e.g., the photoautotrophs.

Unsuccessful attempts have previously been made to combine the desired functionality of direct light-activation in cell-free extracts or purified enzymes for in vitro biocatalysis or bioelectrocatalysis.³⁻⁶ But these strategies have some limitations due to enzyme de-activation in the air or during chemical conversion, without an ability to regenerate the enzyme using the living cell. Other in vivo efforts have been targeted in specific strains of whole non-photosynthetic bacteria,^{7,8} but can limit their applicability due to specific tolerance of the bacteria to inorganic elements and a smaller range of chemicals that can be made. Further, both these processes lack the desired specificity of enzyme activation in living cells, and there is a need to develop a platform technology for such desired living nano-biohybrids for applications beyond solar energy conversion and catalysis, to new avenues in diagnosis and therapeutics. There has been an intensive search for a new method to combine multiple functionalities (e.g., light, voltage, or magnetic field stimulation) of inorganic nanomaterials with the versatility of metabolic networks in living cells, to simply “grow” such hybrid catalysts or convert existing living cells into nanorgs, by the simple addition of inorganic nanomaterials to the cellular medium/water.

The present invention demonstrates the potential of multifunctional living nanorgs by suspending normally non-photosynthetic bacteria in buffered water (in the absence of any sugar) and converting renewable feedstocks like, for e.g., air and CO₂ directly into biofuels and specialty chemicals using these solar-powered factories. Using different core semiconductor nanocrystals or quantum dots (QDs) with tunable bandgap energies (such as CdS, CdSe, InP, and Cu₂ZnSnS₄), and an optimized two monolayer ZnS shell, the chemical binding affinity of zinc is utilized with either a histidine-tagged MoFe nitrogenase in A. vinelandii or Fe—S clusters in hydrogenases and quinones in C. necator, demonstrating the facile formation of nanorgs by self-assembly and simple addition of QDs in the media/buffered water.

These biocatalysts demonstrate high conversion yields to target products (10-100 mg of product/g of dry weight of cells/day) without the utilization of sugar as a source of energy, comparable to or even exceeding (up to −150%) native production levels. The potentially high quantum yields from such light-driven chemical generation (up to 10-20%) depends on the optimization between biocatalyst turnover, incident light-flux, and the amount of light absorbed. Together, these results demonstrate an unprecedented opportunity for development of these nanorgs as renewable sugar-free microbial factories for the production of biofuels and chemicals using sunlight in a scalable process, but also as a means of externally regulating the cellular function of living cells using electromagnetic-stimuli such as light, sound, or magnetic field. In some embodiments, the present invention contemplates nanorg microbial factories including, but not limited to, light-driven renewable biochemical synthesis using quantum dot-bacteria nano-biohybrids.

In one embodiment, the present invention contemplates multifunctional living nanorgs by suspending a range of different normally non-photosynthetic bacteria in buffered water (in the absence of any sugar) and converting renewable feedstocks like, for e.g., air and CO₂ directly into biofuels and specialty chemicals using these solar-powered factories (FIG. 1A). Using different core semiconductor nanocrystals or quantum dots (QDs) with tunable bandgap energies (such as cadmium sulfide (CdS), cadmium selenide (CdSe), indium phosphide (InP), and copper zinc tin sulfide (Cu₂ZnSnS₄)), and an optimized two monolayer zinc sulfide (ZnS) shell, the chemical binding affinity of zinc was utilized with either a histidine-tagged MoFe nitrogenase in A. vinelandii or Fe—S clusters in hydrogenases and quinones in C. necator, demonstrating the facile formation of nanorgs by self-assembly and simple addition of QDs in the media/buffered water. These biocatalysts demonstrate high conversion yields to target products (10-100 mg of product/g of dry weight of cells/day) without the utilization of sugar as a source of energy, comparable to or even exceeding (>150%) native production levels. The high quantum yields from such light-driven chemical generation (13%) depend on the optimization between biocatalyst turnover, incident light-flux, and the amount of light absorbed. While the enzyme turnover and mismatch with the photon flux limits the overall light-to-chemical conversion efficiency (16-20%), better matching the light absorbed in these biohybrids with enzymatic conversion rates through enzyme upregulation or matching incident photon flux can result in improved photon-to-fuel conversion. Together, these results demonstrate an unprecedented opportunity for development of these nanorgs as renewable sugar-free microbial factories for the production of biofuels and chemicals using sunlight in a scalable process, but also as a means of externally regulating the cellular function of living cells using electromagnetic-stimuli such as light, sound, or magnetic field.

### I. Nano-Biohybrid Organisms

A. Core-Shell Quantum Dots.

The core-shell quantum dots (QDs) as contemplated herein were empirically designed and tested. In one embodiment, the disclosed QDs comprise zinc-rich shells for attachment to intracellular proteins of living bacteria. Advantages of the present QDs overcome previous beliefs in the art that incorporation of QDs in living bacteria may cause host bacteria to die. In some embodiments, the present invention contemplates coating the QD with a cysteine zwitterion ligand coating that facilitates uptake of the QD through the cell and improves cell survival.

At least seven different core-shell quantum dots (QDs), with excitations ranging from ultraviolet to near-infrared irradiation were chosen during the development of the present invention based upon the electron emission energy released by the irradiated/illuminated QD for matching a particular energy targeted to the energy level of a particular enzyme site in bacteria. When illuminated by light, these QDs were contemplated to act as a catalyst for increasing enzyme activity of the energy-matched enzyme.

In some preferred embodiments, the production of biofuels comprises using carbon-dioxide (CO₂), water, and with nitrogen oxygen provided by air, as substrates. In some embodiments, the production of biofuels is renewable, from the aspect that the same engineered bacteria strains can be grown in large batches and used to produce the same biofuel.

TABLE 1A
Exemplary matching of irradiation wavelength energy to the reduction potential of the
enzymes in pathways to desired products.

| | Characteristic | | | | | | |
|---|---|---|---|---|---|---|---|
| | Seven CdS@ZnS core-shell quantum dots (QDs) with different wavelength | | | | | | |
| | excitations and examples of associated (linked) enzymes | | | | | | |
| | Quantum-dot | | | | | | |
| | Collectively CZSs | | Collectively CZSe | | | | |
| | CZS1 | CZS2 | CZSel | CZSe2 | CZSe3 | IPZS | CZTS |
| Average | 418 | 437 | 500 | 525 | 580 | 610 | 400 |
| wavelength | | | | | | | |
| (nm) | | | | | | | |
| Color range | Ultraviolet | Blue | Green | Yellow | Orange | Red | Dark |
| (illumination) | | | Contempl | (near- | (near- | (near- | purple |
| | | | ating | infrared | infrared | infrared | |
| | | | using | (NIR)) | (NIR)) | (NIR)) | |
| | | | natural | | | | |
| | | | sunlight | | | | |
| | | | for | | | | |
| | | | illumination | | | | |
| Enzyme | Methyl | Butanediol | Ethylene | Ammonia | polyhydrox | Propanol | Formic |
| linkage | ketones | | | /hydrogen | ybutyrate | | acid |
| | | | | | (PHB) | | |
| Product(s) | Biodiesel: | Gasoline: | ethylene | Fertilizer: | degradable | isopropanol | formic |
| | C₁₁-C₁₅ | 2,3- | (C₂H₄) | ammonia | bioplastics: | (IPA) | acid |
| | methyl | butanediol | | (NH₃) and | PHB | | (FA) |
| | ketones | (BDO) | | hydrogen | | | CH2O2 |
| | (MKs) | | | (H₂) | | | or |
| | | | | | | | CHCHO2 |
| Increase in | +10-50% | +10-50% | +10-50% | +10-50% | up to 150% | 10-50% | — |
| production | biofuel | biofuel | biofuel | biofuel | the PHB | biofuel | |
| compared to | production | production | production | production | yield of | production | |
| their native | | | compared | | wild-type | * purple | |
| conditions | | | to IPZS | | cells | photons | |
| | | | QDs | | up to | showing the | |
| | | | (broader | | around | highest | |
| | | | absorption | | 100 mg | efficiency | |
| | | | spectrum) | | PHB/g | compared | |
| | | | with the | | cell dry | to the blue | |
| | | | C2H4 | | weight | and green | |
| | | | producing | | (CDW) in | photons. | |
| | | | C. necator | | one day. | | |
| | | | strain. | | | | |
| | | | Possibility | | | | |
| | | | of using | | | | |
| | | | sunlight. | | | | |

Azobacteria (A.) vinelandii DJ995, wild-type and genetically modified C. necator strains used for creating nanorgs for providing compounds including but not limited to: H₂, NH₃, FA, C₂H₄, IPA, BDO, MKs, and PHB production.

The generation (accumulation) of the corresponding products are shown in FIG. 15.

### II. Exemplary Compositions And Products

A. Biodiesel: C11-C15 methyl ketones, e.g. C₂H₄, and C₁₁.

Wild-type and genetically-engineered Cupriavidus necator (C. necator) strains for methyl-ketone (MK) production were produced by inserting plasmid pJM20 into C. necator by electroporation as detailed in Muller et. al., Plasmid pJM20 contains the entire MK pathway (‘tesA, fadB, Mlut_11700, and fadM) under the control of BAD promoter. P_{BAD} (araBp; arabinose promoter) is regulated by the addition and absence of arabinose.

Bacteria were grown at 30° C., with shaking at 200 rpm, in the minimal salt media (MSM) supplied with 0.2% fructose and 0.2% glycerol, and the production of MK was realized via induction with 0.2% L-arabinose after 24 h inoculation (count as t=0).

For MK analysis (Muller), 5 ml cell suspension was mixed with 2 ml hexane followed by vigorous shaking for 30 min. After centrifugation (5,000 rpm, 5 min), the upper hexane layer was collected and concentrated (using N2 flow) to 100 1 pd of the hexane layer was injected for GC analysis (180° C. constant oven temperature).

For methyl (MK) production, after induction with L-arabinose (0.2% w/V), 1 ml culture was sampled for assay.

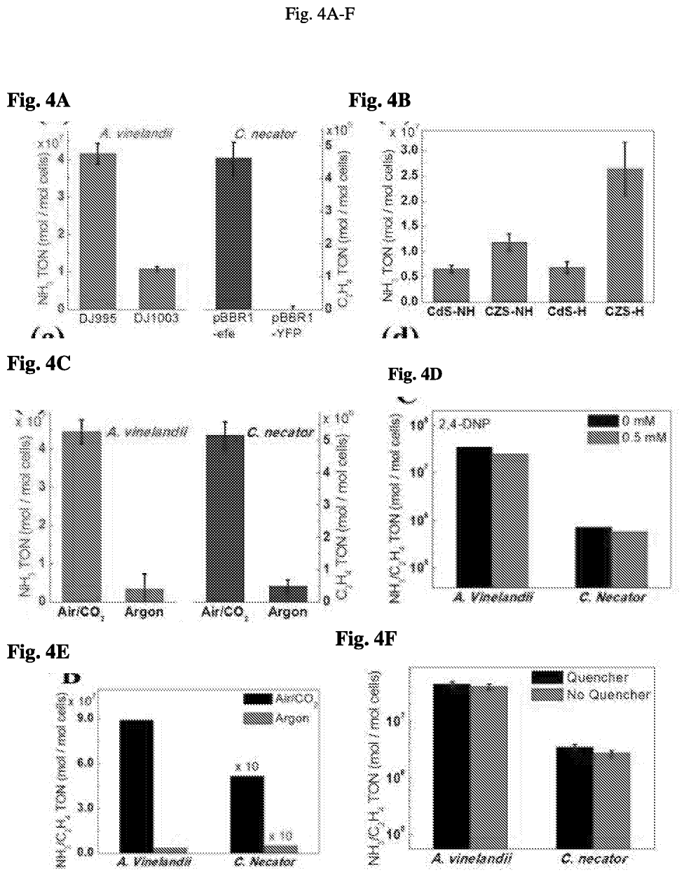

FIG. 4G shows high MK production using IPZS around 125%.

Cupriavidus necator (C. necator) using IPZS. C. necator strains, showing accumulation of C11 products over time, up to 0.015 mM per 80 hours.
Muller, et al., “Engineering of Ralstonia eutropha H16 for autotrophic and heterotrophic production of methyl ketones.” Appl. Environ. Microbiol. 79:4433-4439 (2013).

B. Gasoline: 2,3-butanediol (BDO).

Wild-type and genetically-engineered C. necator strains were utilized using 2,3-butanediol (H16_BO2-20_int) chromosome integration. The strains were grown at 30° C., with shaking at 200 rpm, in the minimal salt media (MSM) supplied with 0.2% fructose and 0.2% glycerol.

C. Alcohols: Isopropanol (IPA).

Wild-type and genetically-engineered C. necator strains were utilized for IPA using (H16_IPAl-10_int) chromosome integration. The strains were grown at 30° C., with shaking at 200 rpm, in the minimal salt media (MSM) supplied with 0.2% fructose and 0.2% glycerol. Additionally, alcohols such as butane diol, were produced for use as gasoline additives and gasoline substitutes.

For IPA and BDO analysis, 1 ml cell suspension was lysed with an ultrasonic probe (3 cycles, 1 min each) followed by centrifuging at 15,000 rpm for 10 min. The supernatant was directly injected (1 fil) into the GC. For IPA, a constant oven temperature (140° C.) was used, whereas programmed temperature ramping (140° C. for 2 min, and 10° C./min ramping to 200° C.) was used for BDO analysis.

D. Ethylene (C₂H₄).

Wild-type and genetically-engineered C. necator strains were utilized for Ethylene (C₂H₄) (pBBRl-efe) plasmid. The strains were grown at 30° C., with shaking at 200 rpm, in the minimal salt media (MSM) supplied with 0.2% fructose and 0.2% glycerol.
Eckert et al, Ethylene-forming enzyme and bioethylene production. Biotechnol Biofuels. 7 (2014).

E. Degradable bioplastics: PHB.

Wild-type and genetically-engineered C. necator strains were utilized for PHB (pBBRl-yfp) plasmid. The strains were grown at 30° C., with shaking at 200 rpm, in the minimal salt media (MSM) supplied with 0.2% fructose and 0.2% glycerol.

FIG. 4 up to around 100 mg PHB/g cell dry weight (CDW) in one day.

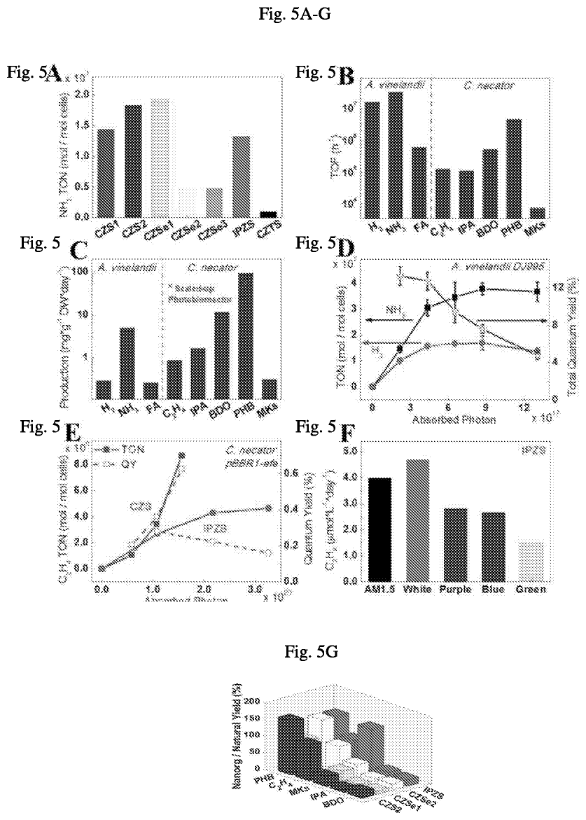

FIG. 5 wild-type C. necator pBBR1-YFP, CZS2 QDs, 400 nm light irradiation, for PHB production.

To demonstrate the efficiency of our sugar-free nanorg system in different light-excited redox reactions, we compared the chemical production yield in our nanorgs (with wild-type and C. necator strains expressing heterologous genes for the production of C2H4, IPA, BDO, or MKs) test with the yield in their natural growth (organolithotropic with fructose/glycerol or formate) conditions (FIG. 4G).

Even for the un-optimized C2H4, IPA, BDO, and MK producing strains, we observed a 10-50% biofuel production, compared to their native conditions. Most notably, when comparing PHB production (a native metabolite produced by C. necator), nanorgs exhibit up to 150% the PHB yield of wild-type cells. Further, the production was easily scaled up from several milliliter tests to liters by simply using a conventional bioreactor with LED panels (FIG. 5).

F. Fertilizer: Ammonia (NH₃) and Hydrogen.

Wild-type and genetically-engineered C. necator strains were utilized for ammonia (NH₃) and hydrogen.

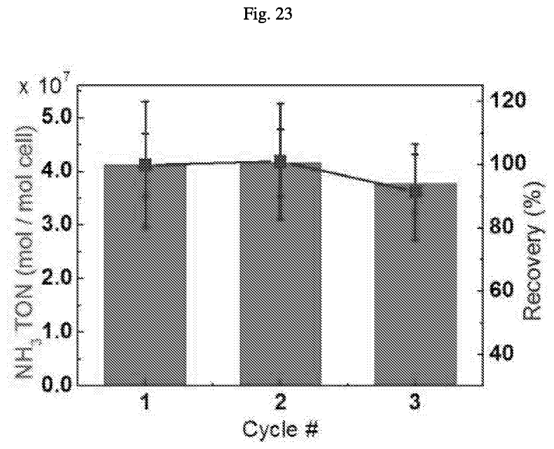

The decrease in quantum efficiency (or the saturation of NH₃/H₂ generation) with higher photon numbers was mainly due to accumulation of the products (functioning as inhibitors), and a replenishment of the reaction media (FIG. 23) showed an almost 100% recovery in NH₃ production.

G. Formic acid (FA).

Generation of formate from CO₂ provides one embodiment of a method for sequestration of carbon from this greenhouse gas. In some embodiments, nanorgs of the present inventions are provided for producing formic acid and derivative chemicals from CO₂. Enzymes include formate oxidases and formate dehydrogenases, e.g. derived from Clostridium carboxidivorans, for catalyzing the reduction of CO₂. Wild-type and genetically-engineered C. necator strains were utilized for Formic acid (FA). In some embodiments, carbon sequestration refers to a process involved in carbon atom capture and storage, e.g. short-term or long-term, of atmospheric carbon, e.g. carbon dioxide (CO₂). In some embodiments, stored carbon may be used for generating compounds as described herein.

With the replacement of LED lights to sunlight, the solar-powered, green product reactor for renewable generation of these targeted biofuels and bioproducts can also be realized commercially.

TABLE 1B
Matching sets of bacteria-construct-enzymes-wavelength-and products.

| | | | | Fertilizer: | |
|---|---|---|---|---|---|
| | Biodiesel: | Gasoline: 2,3- | | ammonia | Degradable |
| | C₁₁-C₁₅ methyl | butanediol | Ethylene | (NH₃) and | bioplastics: |
| Product(s) | ketones (MKs) | (BDO) | (C₂H₄) | hydrogen (H₂) | (PHB) |
| Bacteria species | Wild-type and | Wild-type and | Wild-type and | Azobacteria | |
| | genetically- | genetically- | genetically- | (A.) vinelandii | |
| | engineered | engineered | engineered | DJ.995 and | |
| | Cupriavidus | Cupriavidus | Cupriavidus | DJ1003 | |
| | (C.) necator | (C.) necator | (C.) necator | | |
| | strains, | strains, | strains, | | |
| | e.g. C. necator | e.g. C. necator | e.g. C. necator | | |
| | H16 (strain) | H16 | H16 | | |
| Enzyme pathways | Methyl | Butanediol | Ethylene | Ammonia and | Polyhydroxy- |
| linked to | ketones | | | hydrogen | butyrate |
| photoamplification | | | | | (PHB) |
| Predicted | CdS@ZnS | | — | — | — |
| Quantum-dot | core-shell | | | | |
| | QDs and | | | | |
| | CdSe@ZnS | | | | |
| | core-shell | | | | |
| Product(s) | Biodiesel: | Gasoline: 2,3- | Ethylene | Fertilizer: | Degradable |
| | C₁₁-C₁₅ methyl | butanediol | (C₂H₄) | ammonia | bioplastics: |
| | ketones (MKs) | (BDO) | | (NH₃) and | PHB |
| | | | | hydrogen (H₂) | |
| Predicted | CdS@ZnS core-shell QDs | | CdSe@ZnS core-shell | | |
| Quantum- | CZS1 | CZS2 | CZSe1 | CZSe2 | CZSe3 |
| dot | C = cadmium | | Se = Selenium | | |
| | | | Ultraviolet-visible (UV-VIS) spectra | | |
| Predicted | ultraviolet | blue | green | Yellow | Orange |
| Color range | | | | (near- | (near-infrared |
| | | | | infrared | (NIR)) |
| | | | | (NIR)) | |
| Optimal | | | | | |
| Quantum | | | | | |
| Dot | | | | | |
| Genetic engineering | C. necator- | C. necator- | C. necator- | Transformation | Transformation with |
| | Transformation | chromosome | Transformation | with | pBBR1-YFP plasmid |
| | with Plasmid | integration of 2,3- | with pBBRl-efe | pBBRl-efe | |
| | pJM20 contains | butanediol | | | |
| | the entire MK | (H16_BO2-20_int) | | | |
| | pathway (′tesA, | (operon comprising: | | | |
| | fadB, | alsS(acetolactate | | | |
| | Mlut_11700, | synsthase, | | | |
| | and fadM) | BSU36010) and | | | |
| | under the | alsD(acetolactate | | | |
| | control of BAD | decarboxylase, | | | |
| | promoter. P_{BAD} | BSU36000) from | | | |
| | (araBp; | B. subtilis and | | | |
| | arabinose | sadh(secondary | | | |
| | promoter) is | alcohol | | | |
| | regulated by | dehydrogenase, | | | |
| | the addition | AAA23199.2) from | | | |
| | and absence | C. beijerinckii) | | | |
| | of arabinose. | pBAD | | | |
| | | promoter with araC | | | |
| | | gene was obtained | | | |
| | | from pCM291rfp | | | |
| | | plasmid | | | |
| Product(s) | Biodiesel: | Gasoline: 2,3- | Ethylene | Fertilizer: | Degradable |
| | C₁₁-C₁₅ methyl | butanediol (BDO) | (C₂H₄) | ammonia (NH₃) | bioplastics: PHB |
| | ketones (MKs) | | | and hydrogen | |
| | | | | (H₂) | |
| optimal or | | | CZS-C. necator | | wild-type C. |
| example | | | IPZS-C. necator | | necator-pBBR1- |
| configuration | | | | | YFP-CZS2: FIG. 5 |
| | | | | | in Tab 2 |
| Notes | 10-50% biofuel | 10-50% biofuel | 10-50% biofuel | 10-50% biofuel | exhibit up to 150% |
| | production, | production, | production, | production, | the PHB yield of |
| | compared to | compared to their | compared to | compared to their | wild-type cells. |
| | their native | native conditions | their native | native conditions | up to around 100 |
| | conditions | | conditions | | mg PHB/g cell dry |
| | | | purple photons | | weight (CDW) in |
| | | | showing the | | one day. |
| | | | highest | | |
| | | | efficiency | | |
| | | | compared to the | | |
| | | | blue and green | | |
| | | | photons. | | |
| | | | IPZS QDs | | |
| | | | (broader | | |
| | | | absorption | | |
| | | | spectrum) with | | |
| | | | the C₂H₄ | | |
| | | | producing C. | | |
| | | | necator strain | | |
| | | | Possibility of | | |
| | | | using sunlight | | |

| Product(s) | Isopropanol (IPA) | Formic acid (FA) |
|---|---|---|
| Bacteria species | Wild-type and genetically- | Azobacteria (A.) vinelandii |
| | engineered Cupriavidus (C.) | DJ995 and Wild-type and |
| | necator strains, e.g. C. | genetically-engineered |
| | necator H16 | Cupriavidus (C.) necator |
| | | strains, e.g. C. necator H16 |
| Enzyme pathways linked to | Propanol | Formic acid |
| photoamplification | | |
| Predicted Quantum-dot | IPZS-InP@ZnS | CZTS-Cu₂ZnSnS₄@ZnS |
| | core-shell QDs | |
| | In = Indium | |
| Predicted Color range | Red | Dark purple |
| | (near-infrared (NIR)) | |
| Optimal Quantum Dot | CZS (CZS2 or CZSe1 or | Not available |
| | CZSe2) or IPZS | |
| | But low amounts with each | |
| Optimal color range | Not available | Not available |
| Genetic engineering | C. necator-chromosome | C. necator-Transformation |
| | integration of H16_IPAl- | with pBBRl-yfp plasmid |
| | 10_int | |
| | (operon comprising: bktB(β- | |
| | ketothiolase, H16_A1445) | |
| | derived from C. necator H16; | |
| | ctfAB(Succinyl-CoA | |
| | transferase, AJ000086) from H. | |
| | pylori, adc(acetoacetate | |
| | decarboxylase, CA_P0165) | |
| | from C. acetobutylicum, and | |
| | sadh(secondary alcohol | |
| | dehydrogenase, AAA23199.2) | |
| | from C. beijerinckii. | |
| | pBAD | |
| | promoter with araC gene | |
| | was obtained from | |
| | pCM291rfp plasmid | |
| optimal or example | — | — |
| configuration | | |
| Notes | 10-50% biofuel production, | — |
| | compared to their native | |
| | conditions | |

Choice of QD Core with Desirable Redox Potential.

One initial step towards the development of such living organisms is in vivo site-specific self-assembly, for chemically and energetically coupling the QDs and specific proteins within the synthetic bacteria. To accomplish the desired reaction driven by QD excitation, the reaction center and attachment site for the QD were identified. We designed these nanorgs by appropriately choosing the QD size and material ((Ref. 5, 6) (core-shells, since different materials were required for energetic alignment and chemical coupling/biocompatibility), QD surface charge and ligands (Reference Nos. 7, 8), and the desired site-specific attachment (9). The core QDs were selected using their size- and material-tunable conduction/valence bandedge positions (FIG. 1B) and absorption spectrum, covering the whole ultraviolet-visible-near infrared (UV-VIS-NIR) spectra (FIG. 1A, 1C). To ensure proper energetic alignment and efficient electron injection from the conduction band of photoexcited QDs to selected enzymes (e.g., the molybdenum-iron nitrogenase (MFN) enzyme (Reference Nos. 3, 10, 11) in A. vinelandii, [Fe—S] clusters in the hydrogenases and quinones of C. necator), we conducted in situ experiments with the purified enzyme-QD biohybrids for light-induced catalysis. Our choice of QDs was driven by their strong light absorption in the specific wavelength range (FIG. 1A), easy control of their size (Ref. 12), and tunability of their quantum-confined conduction/valence band positions (Ref. 13).

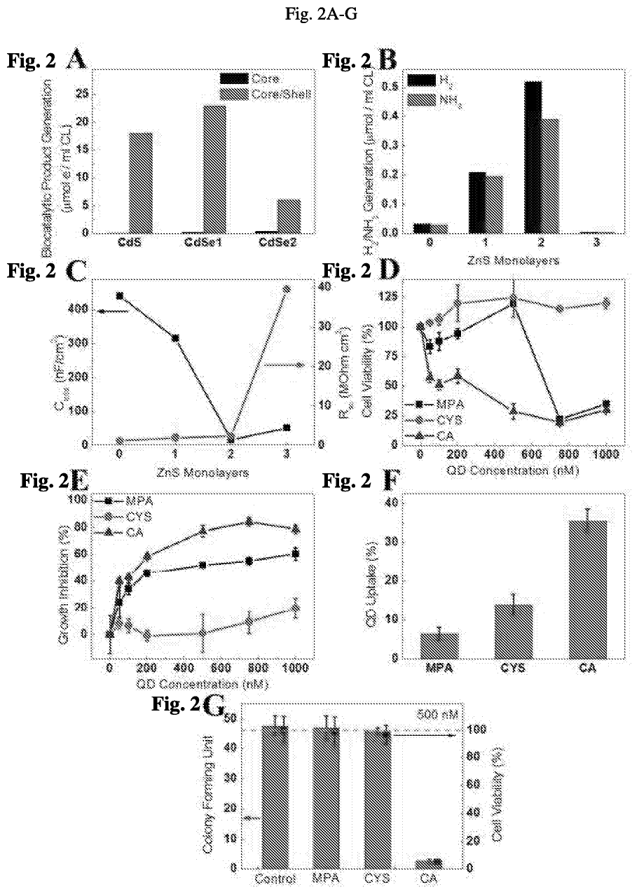

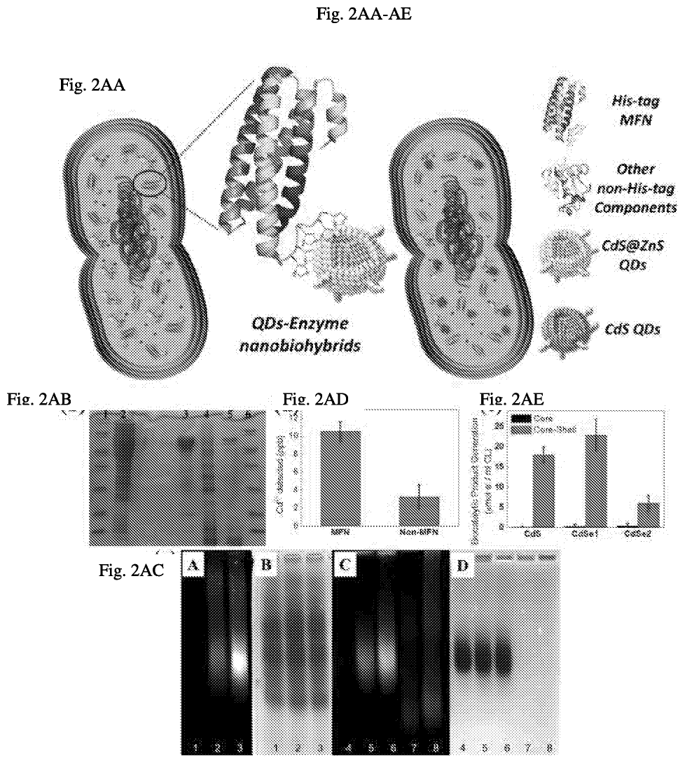

Different sizes of CdS QDs (FIG. 12A) were investigated to absorb the ultraviolet, CdSe QDs (FIG. 12B) to absorb the visible, and InP and CZTS QDs to absorb the near-infrared (NIR) photons. These QDs were specifically chosen to energetically match the reduction potential of the enzymes (Ref. 14, 15), using detailed electrochemical measurements of the QDs (reference Nos. 16-18) (FIG. 1B, FIG. 13A-G, Table S3). While the QDs had the desired electrochemical potential, simply mixing the QDs with the purified enzymes led to low or insignificant photocatalytic activity in QD-enzyme biohybrids (FIG. 2A, MFN), likely due to poor charge injection efficiency of the photogenerated electron (FIG. 12D, FIG. 12E-I, Table S4). Therefore, we developed a robust and tunable platform to evaluate affinity attachment to the desired enzymes, to ensure efficient charge injection and good biocompatibility.

The first step towards the development of such living organisms is in vivo site-specific self-assembly, for chemically and energetically coupling the QDs and specific proteins within the synthetic bacteria. While chemical conversion in biohybrid systems can also be achieved using the formation of intermediates using inorganic catalysis or electrochemistry, followed by utilization of these intermediates by bacteria,⁹⁻¹² direct chemical conversion of inexpensive substrates using light in a bacteria as a platform is still challenging. Such a platform method, envisioned as non-genetically encoded (enzyme regeneration would lead to loss of photosensitization), self-assembled nano-biohybrid enzymes in nanorgs, could even allow selectively triggering cell function for diagnostic evaluation or therapy. To accomplish the desired reaction driven by QD excitation, the reaction center and attachment site for the QD were identified. We designed these nanorgs by appropriately choosing the QD size and material (Ref. 13, 14) (core-shells, since different materials were required for energetic alignment and chemical coupling/biocompatibility), QD surface charge and ligands, (Ref. 15, 16) and the desired site-specific attachment. (Ref. 17). The core QDs were selected using their size- and material-tunable conduction/valence bandedge positions (FIG. 1b) and absorption spectrum, covering the whole ultraviolet-visible-near infrared (UV-VIS-NIR) spectra (FIG. 1a,c). To ensure proper energetic alignment and efficient electron injection from the conduction band of photoexcited QDs to selected enzymes (e.g., the molybdenum-iron nitrogenase (MFN) enzyme (Ref. 3, 18, 19) in A. vinelandii, [Fe—S] clusters in the hydrogenases and quinones of C. necator), we conducted in vitro experiments with the purified enzyme-QD biohybrids for light-induced catalysis. Our choice of QDs was driven by their strong light absorption in the specific wavelength range (FIG. 1a), easy control of their size, (Ref. 20). and tunability of their quantum-confined conduction/valence band positions, (Ref. 21). We investigated different sizes of CdS QDs (FIG. 12a) to absorb the ultraviolet, CdSe QDs (FIG. 12b) to absorb the visible, and InP and CZTS QDs to absorb the near-infrared (NIR) photons. These QDs were specifically chosen to energetically match the reduction potential of the enzymes, (Ref. 22, 23) using detailed electrochemical measurements of the QDs (Ref. 24-25) (FIG. 1b, FIG. 13A-M, Table S3). While the QDs had the desired electrochemical potential, simply mixing the QDs with the cell lysate led to low or insignificant photocatalytic activity in QD-enzyme biohybrids. Therefore, we developed a robust and tunable platform to evaluate affinity attachment to the desired enzymes, to ensure efficient charge injection and good biocompatibility.

### Affinity Binding and Self-Assembly.

Ensuring affinity attachment for chemical coupling and self-assembly between the QDs and the targeted enzymes inside the bacteria (FIG. 2a), (Ref. 27) (19), we screened common biocompatible QD shell materials using large particles for their selectivity in attaching to specific sites on our target proteins. Here, metal-histidine affinity was investigated. We conducted protein-binding tests using the cell lysate (produced from genetically-engineered A. vinelandii DJ995 strain, which contains a His-tagged MFN¹⁸) with ZnS and CdS, by incubating them in room temperature followed by eluting the trapped components with imidazole. SDS-PAGE with Coomassie staining for the released protein indicates single band (FIG. 2b) with ZnS, showing its selective binding to His-tagged MFN. And no such selectivity was seen with CdS. We have also demonstrated such site-specific binding by using fluorescent CdSe@ZnS core-shell QDs with the cell lysate, by running the mixture through the agarose gel using electrophoresis. Majority of observed fluorescence (FIG. 2c) and cadmium (FIG. 2d) detected from His-tagged MFN band indicates the specific binding between them. Since simply mixing QDs with the desired electrochemical potential with the cell lysate led to low or insignificant photocatalytic activity in QD-enzyme biohybrids (FIG. 2e, MFN), we evaluated potential role for a shell around the QD core. By changing the shell thickness around the QDs, we observed that the low photocatalytic activity of the core could be due to poor charge injection efficiency of the photogenerated electron, as shown with strong reduction of sub-bandgap/trap state recombination (FIG. 12d), clear decrease in interfacial charge injection and capacitance due to trapped charges (FIG. 13e-i) and slower open-circuit potential decay (FIG. 13i, compiled data in Table S4). Using histidine (His)-tagged MFN (10) (produced from the genetically engineered A. vinelandii bacteria) as a test case, strong and selective binding was seen from the cell lysate (CL) with ZnS, as shown in the single stained band from protein electrophoresis (FIG. 13A). However, the CdS particles (core material) showed weaker and non-specific binding to numerous proteins.

To maintain the desired energetic coupling of photoelectron production in CdS and the site-selective MFN binding with ZnS, CdS@ZnS core-shell quantum dots were designed to selectively trap the His-tagged MFN from the cell lysate (prepared from the A. vinelandii DJ995 bacteria) and conduct light-induced in situ redox reactions.

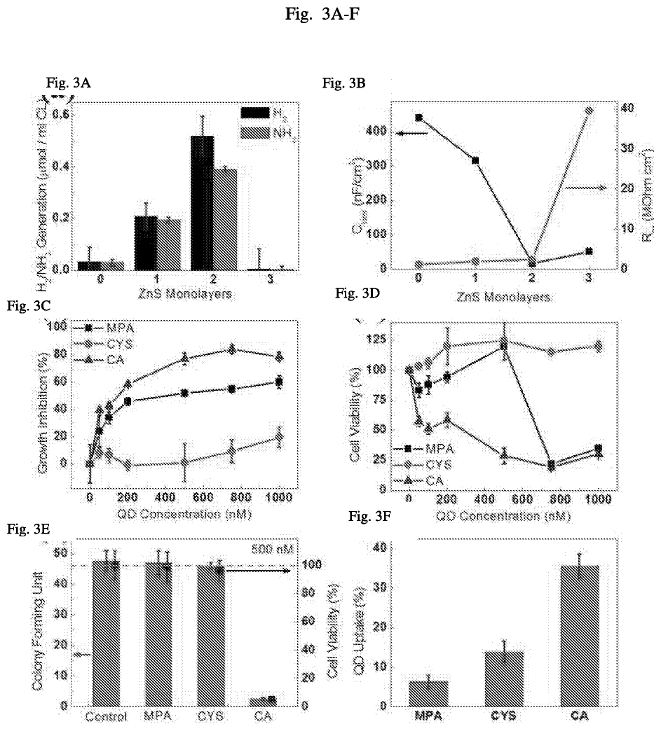

Optimizing the injection of photogenerated electrons from different core QDs to the enzyme active site, the core-shell QDs were synthesized using a layer-by-layer deposition technique (6¹⁴), with precise control of the ZnS shell thickness (FIG. 12C, FIG. 12D, Table S1). While increased shell thickness ensured site-selective attachment, biocompatibility, improved charge injection of the photogenerated electrons to the active enzyme site, and increased surface passivation reducing surface states/defects; thick ZnS shells also served as a barrier for charge injection from different QDs cores. To realize an optimal design, we coupled CdS@ZnS QDs with nominal x monolayer (ML) ZnS shells (CZS-xML, x=0 to approximately 3, capped with 3-mercaptopropionic acid (MPA) ligand) with the cell lysate and tested their photocatalytic efficiency in H₂ and NH₃ production. We used light-induced (400 nm) H₂O and N₂—H₂O reduction in pH=7.4 media, utilizing either L-ascorbic acid or HEPES as a sacrificial agent. Here, we observed a significant enhancement of H₂ and NH₃ generation with the QDs-cell lysate biohybrids, generating a maximum of 615 nmol/ml cell lysate for H₂ production from H2O reduction and 527/337 nmol/ml cell lysate H₂/NH₃ production from N₂—H₂O reduction (net production, with correction using the reaction phase containing QDs, in 30 min) using the biohybrids with 2 ML ZnS-coated QDs (FIG. 3a, FIG. 13B, FIG. 13C). As a comparison, QDs without MFN attachment (zero ML shell) or with thick shells (three MLs) show a negligible yield. The affinity binding of His-tagged MFN to zinc-rich QDs surface was also confirmed by control experiments (FIG. 13D-F), where the addition of imidazole (coordinates with Zn²) or increasing media acidity (protonates histidine) inhibits such interactions (19²⁷) and hence no H₂ or NH₃ production was observed (same as the systems with QDs as control). Optimal design of a 2 ML thick CdS@ZnS QD-MFN biohybrid was also evident by electrochemical impedance spectroscopy (FIG. 3b, FIG. 13G, FIG. 13H small total capacitance and charge transport resistance), open-circuit potential decay (FIG. 13I, Table S4, reduced non-radiative charge recombination) and photoluminescence (FIG. 12D, removal of surface states).

### QD Biocompatibility and Ligands.

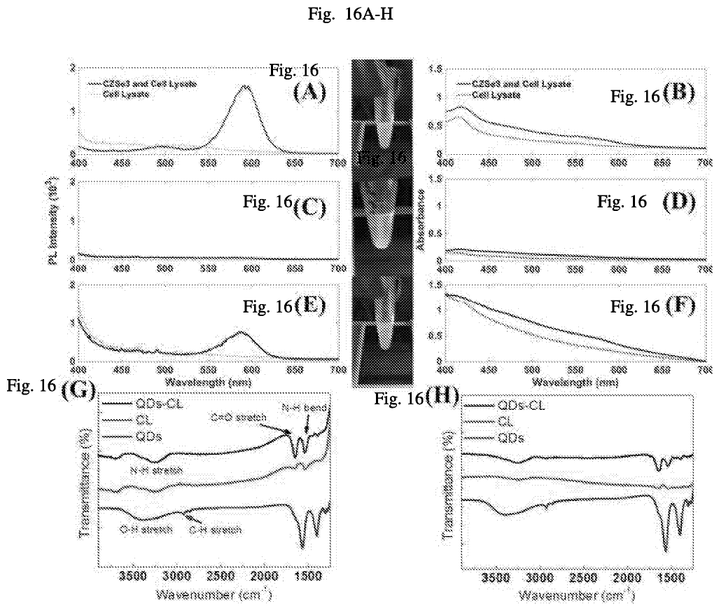

The chemical attachment of QDs (capped with 3-mercaptopropionic acid or cysteine) to the proteins was demonstrated by Fourier-transformed infrared (FTIR) spectra of coupled QDs-CL after washing (to remove any unbound/weakly bound cellular component in CL, FIG. 16G, FIG. 16H), showing characteristic peptide vibration modes and the disappearance of O—H stretching in the QDs-protein complex. Binding of cellular proteins to the QDs was also confirmed by UV-VIS analysis of QDs-CL, purified using centrifugation (FIG. 16A-F). To further investigate the affinity of QDs binding QDs-CL mixtures were separated by gel electrophoresis. Here, highly fluorescent ZnS-capped QDs were detected mainly from the His-tagged MFN band (FIG. 2Ac), where selective QD attachment was confirmed by elemental analysis (FIG. 2Ad) of the His-tagged MFN band and the non-His-tagged protein bands. To further demonstrate the causative link between affinity attachment and the photogenerated electron driven catalytic reduction of air-water using nanorgs, we conducted measurements using a different strain of A. vinelandii (DJ1003) and C. neactor (pBBRl-YFP), which produces apo-nitrogenase enzyme lacking the MoFe cofactor and pBRl-efe plasmids (targeted enzymes), and observed significant decrease in generation of products (FIG. 3A). This demonstrates the importance of simultaneous optimization of surface tuning, photophysics, and charge tunneling (across QD-shell) in designing highly efficient biohybrids. Using this evidence for affinity attachment, in vitro studies with QDs-cell lysate (to decouple the role of QD transport), we demonstrated the simultaneous optimization of surface tuning, photophysics, and charge tunneling (across QD-shell) in designing highly efficient biohybrids.

Another aspect of the QD surface related to nanorg development was the ligand and the overall charge on QDs (9, 20-23^{17,28-31}), which affected their biocompatibility, viability, and the uptake of designed QDs for intracellular self-assembly. Several attempts to combine the desired functionality of QDs with the synthetic versatility of the designed bacteria have relied on cell-free extraction of the enzymes and their coupling with QDs. These approaches showed limitations such as loss of enzyme activity or even deactivation (3, 10, 24^{3-6,18,32}) low enzyme concentrations, issues with scale-up, and low-activity and TON for chemical conversion. Using QDs capped with similar-sized ligands as well as different surface charge (negatively-charged MPA, positively-charged cysteamine (CA), and zwitterion cysteine (CYS)), we tested the viability of bacteria with QDs using three different methods. First, when monitoring cell growth by optical density, we observed that bacteria with CYS-capped QDs exhibited growth similar to no treatment, MPA-capped QDs impaired growth moderately, and CA-capped QDs strongly inhibited growth, especially at high concentrations (FIG. 3c, FIG. 18a-d). Under light irradiation with non-growing nanorg cells in the photocatalytic media, cell viability was almost unaffected (FIG. 18j-l)) with both the CYS-capped QDs and the low concentration MPA-capped QDs. With the higher concentration MPA-capped nanoparticles and even the low (50 nM) concentration of CA-capped QDs, a significant decrease in cell viability was observed. Low cell viability could render the cell unable to remove the oxygen in the air and cause deactivation of the oxygen-sensitive enzymes ((reference Nos. 3, 10, 24). This leads to low NH₃ production in light-induced air-water reduction using the QDs-living bacterial biohybrids and can be checked by performing the photocatalytic test in a different atmosphere. We observed a significant decrease (by one magnitude) of NH₃ yield in air-FhO reduction compared to pure N₂ (oxygen-free)-H₂O (FIG. 22c, d). The second test utilized on the nanorgs to measure the cell viability was the resazurin dye assay (13), performed after the photocatalytic test, which also demonstrated a high cell viability with both zwitterion and (low concentration) negatively-charged QDs (FIG. 22c, d). A more detailed investigation with colony forming unit analysis (CFU) also showed the same results (FIG. 3e), with the highest viability for zwitterion and negatively-charged QDs. Using the cell size of A. vinelandii (2.7-6.6×10¹⁵ liters), the cell viability, even at high QD concentrations (>1000 nM), and modest uptake with zwitterion QDs (approximately 14%, FIG. 2 FIG. 3f), we estimated approximately 10⁴ QDs per cell, which guarantees enough QDs in the cell to couple with available MFN enzymes. QD uptake by the cells was also visualized using laser-scanning confocal microscopy, showing a uniform distribution of QDs inside the bacteria (FIG. 18A-L). Uniform distribution of QDs throughout the cells (monitored using fluorescent QD in a confocal microscope) proves intracellular QD uptake, enabling self-assembly of QDs with enzymes. While the cellular uptake with positively charged CA-capped QDs was much higher than both negative or zwitterions ligands with similar sizes, the strong non-specific attachment of QDs to negatively-charged cell organelles (like, for e.g., DNA, RNA, proteins) could be responsible for the low cell viability, especially at high CA-capped QD concentrations.

Following the design and self-assembly of appropriate QD-bacteria biohybrid nanorgs (QDs with two monolayers thick ZnS shell, capped with cysteine ligand), we tested their ability to fix the energy of incident light-photons into specific chemical bonds using renewable and inexpensive substrates/feedstocks, like, for e.g., air, water, and CO₂. Control experiments with either no QDs, light irradiation, or the cells, show no NH₃ or C₂H₄ production in A. vinelandii and C. necator strains (FIG. 22A, FIG. 22B). The same tests conducted under lack of available substrate/feedstock (argon atmosphere, so no N₂ or CO₂ substrate available) also showed no product generation (FIG. 3B, FIG. 22C). These ruled out the detection/production of NH₃ from either a cellular source (nitrogen-containing proteins, amino acid, DNA, etc.) or the media. Further controls included lack of product generation without chemical coupling (FIG. 2A), sudden turn-off of light-driven chemical generation by decoupling the QD-enzyme (FIG. 18B, FIG. 18C), and lack of significant product formation with synthetic constructs which lack the site for affinity QD attachment or charge injection of photogenerated electrons to drive biocatalysis (FIG. 3A).

### Light-Triggered Enzyme Activation

Further experiments also demonstrated the fixing of incident photon energy, as chemical fuels provide detailed measurements of light-intensity dependent chemical generation (FIG. 23E), a direct correlation between cell viability of the nanorgs and product yield (FIG. 23B-D), and improvement of product formation with nanorg cell optical density leading to higher light absorption (FIG. 23A). The addition of an ionophore (such as 2,4-dinitrophenol (2,4-DNP)) into the photocatalytic system further verified chemical generation via direct transfer of photogenerated electrons from QDs to the enzyme. Continuous chemical generation with a minor decrease in yield (FIG. 3C, FIG. 22E) indicates a direct electron injection from QDs to enzyme instead of an ATP-dependent charge transfer via NADPH, for NH₃/C₂H₄ production. To further verify strong chemical and electrochemical coupling between QD-enzyme biohybrids in nanorgs, the addition of sacrificial donors had a minor effect on the chemical generation (FIG. 3D), whereas a much stronger quenching of photogenerated charge carriers and chemical generation would be expected in case of uncoupled QDs or redox shuttles. Intracellular uptake and chemical attachment in nanorgs was also verified using larger nanoparticles, which cannot penetrate the bacterial cell membrane. By using these larger “microparticles” of the same materials, negligible product yields were observed (section: Photocatalytic test with the microparticles), providing further evidence for the importance of affinity binding to specific sites as a necessary step for such photon-driven biocatalysis.

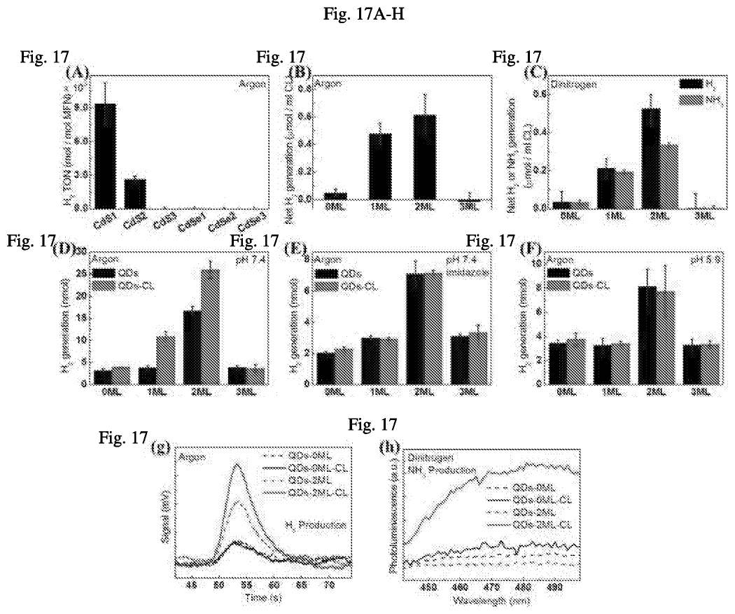

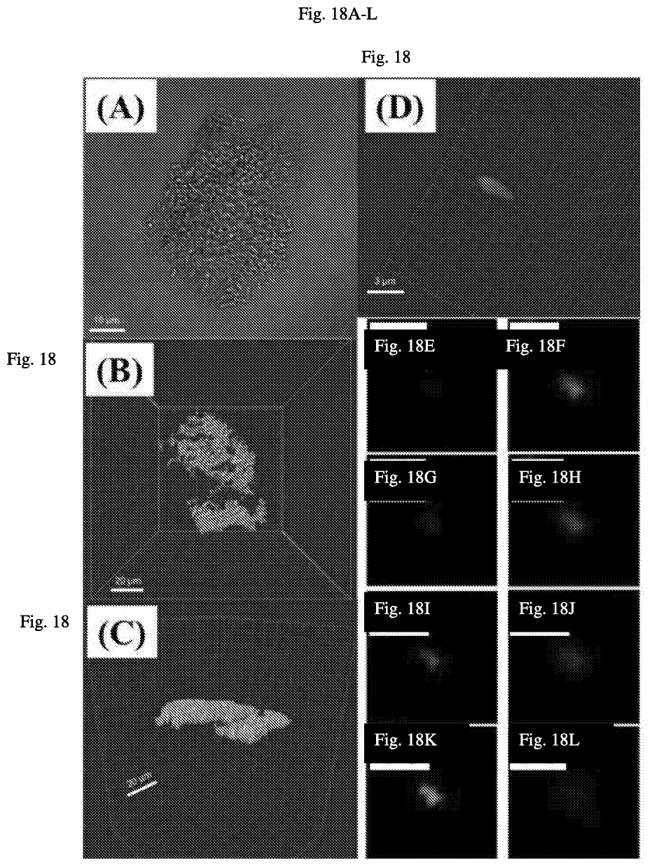

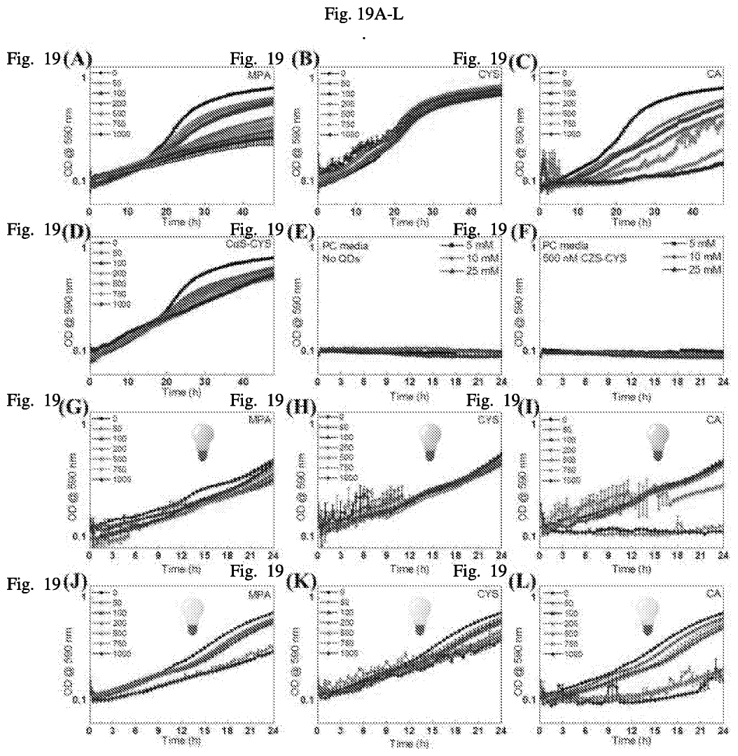

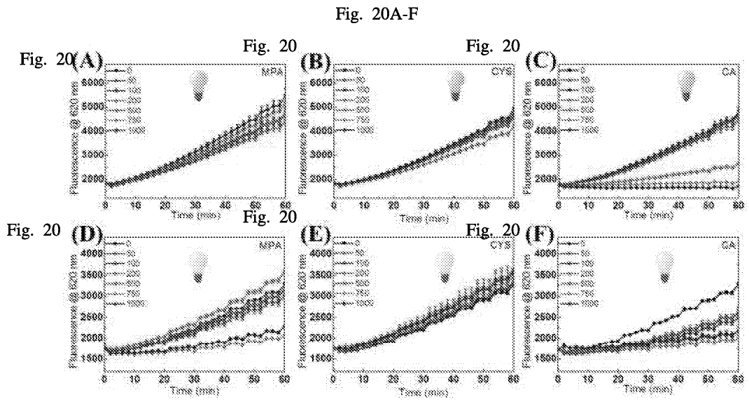

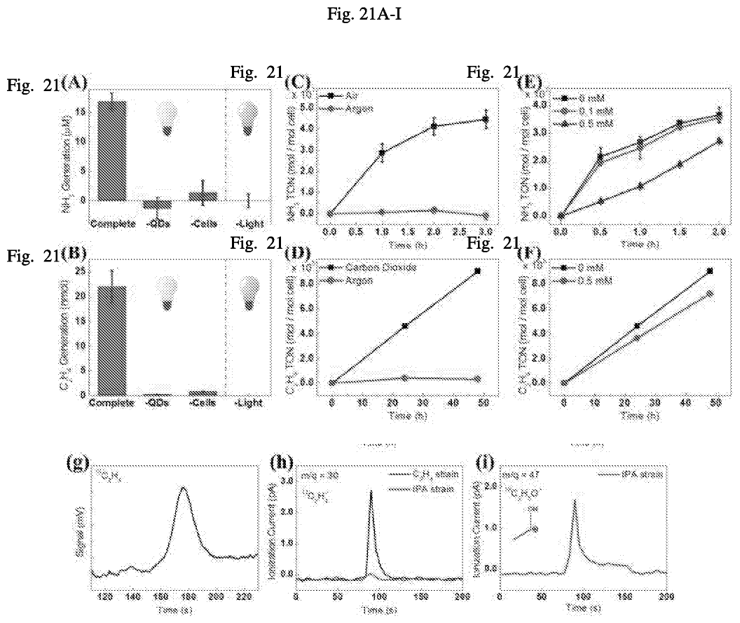

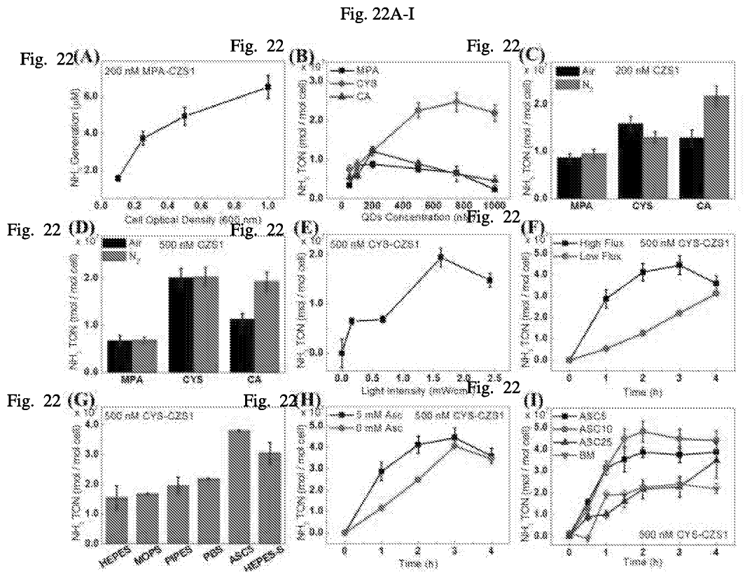

Following the design and self-assembly of appropriate QD-bacteria biohybrid nanorgs (QDs with two monolayers thick ZnS shell, capped with cysteine ligand), we tested their ability to fix the energy of incident light-photons into specific chemical bonds using renewable and inexpensive substrates/feedstocks, like, for e.g., air, water, and CO₂. Control experiments with either no QDs, light irradiation, or the cells, show no NH₃ or C₂H₄ production in A. vinelandii and C. necator strains (FIG. 21a,b). Using strains with decreased nitrogen fixation capability (A. vinelandii DJ1003, which produces a His-tagged apo-nitrogenase with no MoFe cofactor) and no ethylene pathway (C. necator pBBR1-YFP, with the lack of “efe” gene for ethylene production), significant decrease of ammonia or no ethylene production (FIG. 4a) was seen. Similar ammonia production tests were also performed by using CdS QDs (without ZnS shell) and an A. vinelandii strain producing non-His-tagged MFN (details in the supporting information), where no site-specific zinc-histidine binding was utilized. In these controls, the ammonia yield also decreased significantly (FIG. 4b) to approximately ⅓, compared to the zinc-histidine binding. The same tests conducted under lack of available substrate/feedstock (argon atmosphere, so no N₂ or CO₂ substrate available) also showed no product generation (FIG. 4c, FIG. 21c). Isotope (¹³C) labelling tests (mass spectra, FIG. 21g,h,i) also proves biochemical and biofuel production from ¹³CO₂ as the substrate. These ruled out the detection/production of NH₃ and other biofuels from either a cellular source (proteins, amino acid, DNA, etc.) or the media. Further controls included lack of product generation without chemical coupling (FIG. 2e), sudden turn-off of light-driven chemical generation by decoupling the QD-enzyme (FIG. 17e, f), and lack of significant product formation with synthetic constructs which lack the site for affinity QD attachment or charge injection of photogenerated electrons to drive biocatalysis (FIG. 4a). Further experiments also demonstrated the fixing of incident photon energy, as chemical fuels provide detailed measurements of light-intensity dependent chemical generation (FIG. 22e), a direct correlation between cell viability of the nanorgs and product yield (FIG. 22b-d, see descriptions herein for detailed explanations), and improvement of product formation with nanorg cell optical density leading to higher light absorption (FIG. 22a). The addition of an ionophore (such as 2,4-dinitrophenol (2,4-DNP)) into the photocatalytic system further verified chemical generation via direct transfer of photogenerated electrons from QDs to the enzyme. The continuous chemical generation with a minor decrease in yield (FIG. 4d, FIG. 21e) indicates a direct electron injection from QDs to enzyme for NH₃/C₂H₄ production. Furthermore, the addition of sacrificial donors had a minor effect on the chemical generation, which strengthens the conclusions from control experiments pointing to the role of QD-enzyme binding (FIG. 4e). Intracellular uptake and chemical attachment in nanorgs was also verified using larger nanoparticles, which cannot penetrate the bacterial cell membrane. By using these larger “microparticles” of the same materials, negligible product yields were observed, providing further evidence for the importance of affinity binding to specific sites as a necessary step for such photon-driven biocatalysis.

After confirming the light-induced redox reaction by direct electron injection in nanorgs, we optimized the conditions further for chemical generation via enzyme activation, utilizing factors including bacterial cell density, irradiation intensity, sacrificial donor concentration (to improve) TOF of enzymes), and QD capping ligands and concentration (FIG. 22A-I). We observed a clear correlation between NH₃ production and cellular uptake and cell viability in A. vinelandii DJ995 (also confirmed by parallel experiments in air and a pure nitrogen atmosphere, FIG. 22C, FIG. 22D). Using these conditions (detailed as described herein), we extended our test with various QDs covering absorption spectrum from near-UV, visible, and near-IR.

These QDs form nanorgs with a high TON for NH₃ (FIG. 5a), up to 10⁷ (with CZS, CZSe1, and IPZS QDs).

The lower TON for the other nanorgs is mainly due to an unfavorable redox potential match or low absorptivity (especially for CZTS). We also demonstrated the flexibility of the nanorg platform by utilizing direct electron transfer to the living bacteria for different desired fuel generation. By coupling the QDs with a variety of bacterial strains (A. vinelandii DJ995, wild-type and genetically modified C. necator strains), we were able to create nanorgs for H2, NH₃, FA, C₂H₄, IPA, BDO, MKs, and PHB production (FIG. 5b), with high turnover frequencies up to 10⁷ per hour. These nanorgs were able to accumulate different amounts of these chemicals (FIG. 5c), with up to around 100 mg PHB/g cell dry weight (CDW) in one day. Production of H₂ and NH₃ with time (FIG. 5d, represented as absorbed photon) shows a maximum internal quantum efficiency of about 13.1%. The decrease in quantum efficiency (or the saturation of NH₃/H₂ generation) with higher photon numbers was mainly due to accumulation of the products (functioning as inhibitors), and a replenishment of the reaction media (FIG. 23) showed an almost 100% recovery in NH₃ production. As a comparison, with lower absorbed photons, C₂H₄ production (FIG. 5e, with CZS QDs) kept increasing with no sign of the saturation effects seen in the NH₃ production (less inhibition by C₂H₄ in the gas phase). However, high photon input (with IPZS QDs) does not help increase the yield, and the quantum yield for C₂H₄ production is about one magnitude lower than the NH₃. This could be due to the un-optimized C₂H₄ producing C. necator strains (where a small portion of electrons are converted to C₂H₄ due to the complex regulation of native pathways required to produce substrates for the heterologously expressed ethylene-forming enzyme (Reference No. 25)), compared to the N₂-fixing A. vinelandii strains for NH₃ production.

### Characterization of Biocatalytic Conversion.

Optimizing the turnover number of light-driven chemical generation using nanorgs, we investigated the turnover frequency of the enzymes by comparing the photon flux for activation of the self-assembled QD-enzyme biohybrids, activated using light for converting photons to chemical bonds. First, we estimated the photon flux per biohybrid enzyme, by determining the light flux (approximately 1.6 mW/cm²) and using photon energy (approximately 2 eV) to obtain photon flux (5×10¹⁵ photons/sec/cm²). Using the optical density of the cells and the resulting nanorgs per unit area (approximately 10⁸ nanorgs/cm²) and the estimated number of biohybrid enzymes (approximately 10,000 biohybrid enzymes/nanorg), we obtained the photon flux per biohybrid enzymes (approximately 5000 photons/biohybrid enzyme/sec). Comparing this to enzyme turnover (for MFN enzyme, 3000 nmol/mg/sec for 250 kDa enzyme≈(approximately equal to) 750/sec), we estimated an approximately 6-fold incident photons/enzyme turnover, thereby highlighting the mismatch between the number of biohybrid enzymes available for chemical generation in the nanorgs and the high incident flux of light. This would limit the maximum possible quantum efficiency to approximately 16-20%, further highlighting the high efficiency of enzyme activation using light (13% in our experiments), and the resulting biofuel and chemical generation using nanorgs. This could be optimized by utilizing synthetic biology tools to upregulate the enzymes, thereby coupling the photon flux to enzyme turnover, or reducing the photon flux. To study photon-energy related fuel production, we chose the nanorgs made from IPZS QDs (broader absorption spectrum) with the C₂H₄ producing C. necator strain, using the same test under different light sources (AM 1.5 (with 400 nm long pass filter), white, purple, blue, and green LED). All these light sources are able to excite the nanorgs for C₂H₄ production (FIG. 5f), with the purple photons showing the highest efficiency compared to the blue and green photons. Moreover, high production with AM 1.5 irradiation indicates the possibility of using sunlight as an energy source to power the nanorg factories for solar fuel production. To demonstrate the efficiency of our sugar-free nanorg system in different light-excited redox reactions, we compared the chemical production yield in our nanorgs (with wild-type and C. necator strains expressing heterologous (exogenous) genes for the production of C₂H₄, IPA, BDO, or MKs) test with the yield in their natural growth (organolithotropic with fructose/glycerol or formate) conditions (FIG. 4G, FIG. 5g). Even for the un-optimized C₂H₄, IPA, BDO, and MK producing strains, we observed a 10-50% biofuel production, compared to their native conditions.

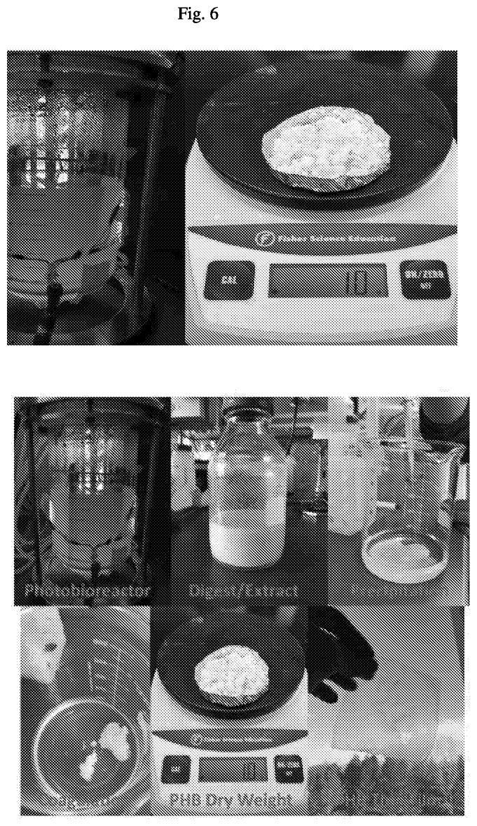

Most notably, when comparing PHB production (a native metabolite produced by C. necator), nanorgs exhibit up to 150% the PHB yield of wild-type cells. Further, the production was easily scaled up from several milliliter tests to liters by simply using a conventional bioreactor with LED panels (FIG. 5, FIG. 6). Using the wild-type PHB producing strains in the photobioreactor (approximately 4 L) with a similar condition (as in the 5 ml test scale), we were able to obtain gram-scale production of PHB, with no noticeable change of yield compared to small-scale test. The scaled-up production indicated the potential capability of our system in further scaling up, from lab scale (several liters) to a pilot scale plant (approximately 1000 L), and eventually even to a commercial level (>40,000 L) with little change of the configuration. With the replacement of LED lights to sunlight, the solar-powered, green product reactor for renewable generation of these targeted biofuels and bioproducts can also be realized commercially. These results demonstrate the potential application of our nanorgs platform in direct, scalable, and renewable generation of fuels from sunlight, using air or CO₂, and the ability to activate a range of targeted enzymes externally, using electromagnetic stimuli.

Biochemical conversion can also be realized by using electricity. In electro-biochemical synthesis,³⁴ the bacteria are immobilized on a modified electrode, which can inject electrons for downstream enzymatic reactions. The two processes: electro-biochemical synthesis and photo-biochemical synthesis, can be compared as photovoltaic driven-electrochemical synthesis and photocatalysis. The electro-biochemical approach circumvents several requirements of QDs-bacteria interactions, including QD uptake and low QD toxicity, making the design easier. However, electro-biochemical synthesis is limited to some special microbes that can exchange electrons with electrodes, and the requirements of immobilization and using redox mediators. Furthermore, there are no mechanisms in specifically targeting a desired enzyme or metabolic pathway for selective biochemical conversions. Therefore, photo-biochemical synthesis by using a designed QD-microbe biohybrid can offer additional advantages over other methods.

In conclusion, we have demonstrated a method for the formation of a living QD-bacterial nano-biohybrid nanorgs via the design of appropriate QDs and facile mixing, self-assembly, and affinity binding to the desired enzymes. Using a range of different light-absorbing QDs and targeted enzymes in different bacterial strains, we demonstrate the broad applicability of the proposed direct activation of the enzyme and the generation of biofuels and chemicals from non-photosynthetic microbes by simply suspending them in buffered water and bubbling air and/or CO₂. Large turnover numbers and frequencies along with the high quantum efficiency for the direct conversion of light into chemicals were obtained for biofuel precursors and specialty chemicals including MKs, BDO, H₂, IPA, NH₃, FA, and PHB, demonstrating the potential and a possible application of the proposed method.

The biochemical conversion yields of the proposed method to simply utilize formed nanorgs, CO₂ and water, in absence of sugar, even exceeded the natural yields in growing media (>150%), limited by the enzyme turnover. While maximum achievable quantum efficiency of light-to-chemical conversion can be 16-20% in the nanorgs, due to slow enzymatic conversion (˜1.3 msec) compared to absorbed light flux in the spontaneously self-assembled nano-biohybrids, high conversion efficiencies of light-activated chemical conversion (13%) highlights the potential of such simple platform for making self-assembled nanorgs and ability of electromagnetic enzyme activation. Further, such catalytic conversion can be optimized by upregulating the desired enzymes using tools in synthetic biology, and better matching the incident photon flux with the achievable turnover of the enzymes, for improved energy conversion.

This technique can easily be scaled up; be extended by improved screening for affinity binding to the proteins, expanding the scope of making nanorgs with other prokaryotes and eukaryotes; testing theories in molecular biology; and developing new diagnostic and therapeutic methods using other external stimuli, e.g. sound waves; magnetic field, etc.

TABLE S1
Total Cd and Zn (in ppb) determined from ICP-MS and
determination of the thickness of the ZnS shell (Real layer number).

| | | Total | | Real |
|---|---|---|---|---|
| CdS@ZnS | Total Cd (ppb) | Zn (ppb) | Dtotai (nm) | layer (ML) |
| OML | 469720 | 1427 | — | — |
| 1 ML | 94441 | 25677 | 3.94 | 0.6 |
| 2 ML | 38230 | 46066 | 4.91 | 2.2 |
| 3 ML | 243618 | 577668 | 5.74 | 3.5 |

TABLE S2
Core-shell (ZnS, 2ML) extinction coefficient at individual specific wavelengths.

| | CZS1 | CZS2 | CZSel | CZSe2 | CZSe3 | IPZS | CZTS |
|---|---|---|---|---|---|---|---|
| wavelength | 418 | 437 | 500 | 525 | 580 | 610 | 400 |
| (nm) | | | | | | | |
| Extinction | 385,676 | 573,697 | 68,488 | 67,792 | 120,000 | 1,272,300 | ~10⁴ |
| coefficient | | | | | | | |
| (M⁻¹ cm⁻¹) | | | | | | | |

TABLE S3
Bandedge and bandgap information of CdS and CdSe QDs obtained
from optical (UV-VIS) and electrochemical (DPV) measurement.

| | E_{CB} (V) | E_{VB} (V) | E_{g, ec} (eV) | E_{g, op} (eV) | D (nm) |
|---|---|---|---|---|---|
| CdSl | −0.83 | 2.25 | 3.08 | 3.06 | 3.55 |
| CdS2 | −0.75 | 2.20 | 2.95 | 2.94 | 4.17 |
| CdS3 | −0.61 | 2.21 | 2.82 | 2.79 | 5.06 |
| CdSel | −0.72 | 1.89 | 2.61 | 2.50 | 2.30 |
| CdSe2 | −0.53 | 1.85 | 2.38 | 2.38 | 2.59 |
| CdSe3 | −0.31 | 1.88 | 2.19 | 2.07 | 4.58 |

Note:
E_{CB}, E_{VB} (vs. NHE) are the conduction band and valence band position, respectively, from DPV measurements. E_{g, ec} is the electrochemical bandgap determined from the conduction and valence band position (E_{g, ec} = E_{VB} − E_{CB}). E_{g, op} is the optical bandgap from UV-VIS measurements (E_{g, op} (eV) = 1239.8/A (nm), where A is the wavelength of the first excitation peak). D is the diameter of the nanoparticles determined from the optical bandgap.

### IV. Cellular Enzyme Preparation and Characterization.

A. A. vinelandii DJ995 Bacteria Growth and Cell Lysate Preparation.

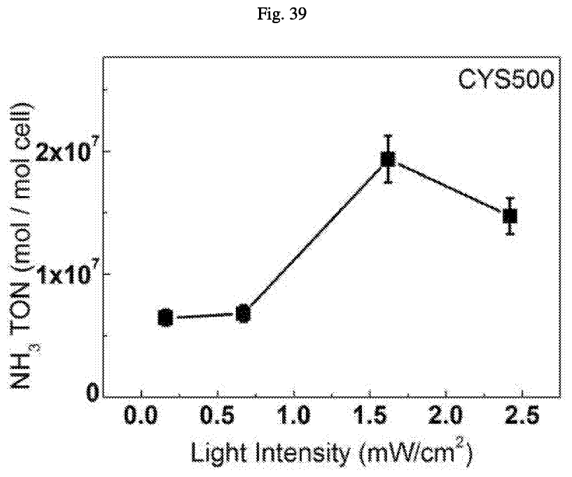

In one embodiment, A. vinelandii DJ995 bacteria (wild type, with a histidine tagged MoFe nitrogenase on the C-terminus of the a-subunit), producing MoFe nitrogenase with 7× histidine tag on the C-terminal of the a-subunit was used. The cells were grown (31 s) in a nitrogen-free modified Burk media (500 ml for each batch, in a 2 L Erlenmeyer flask) with air bubbling (approximately 1 LPM) and shaking (approximately 300 rpm) for 24 hours (to an optical density of −1.5 at 600 nm). The resulting cells (dark brown color as shown in FIG. 39B, left) were centrifuged (precipitated) at 6,000 rpm and washed twice with equivalent buffer (25 mM Tris-HCl, 0.5 M NaCl, pH 7.9) and stored at −80° C. The cell lysate was prepared using ultrasonication (anaerobic and 4° C. condition). The frozen cells (approximately 8 gram) were anaerobically thawed and suspended in 16 ml fully degassed equivalent buffer (with 0.2 mM PMSF and 2 mM sodium dithionite), and the resulting cell suspension was ruptured in a side-arm test tube (flushed with UPC argon) using an ultrasonic probe at full power (Fisher Sonic Dismembrator Model D100, with 1/1 min sonication/rest for 10 cycles). The cell suspension was cooled using an ice-water bath and the sonication was taken at 1 min sonication and 1 min rest cycle for 10 cycles. The cells were anaerobically transferred to an argon-flushed ultracentrifuge tube (Beckmann Coulter #355618) and centrifuged at 30,000 rpm (Beckmann Coulter L8-70M, with the Ti-45 rotor) for 30 min. The dark brown supernatant (FIG. 39B) was dropped into liquid nitrogen (LN₂) using an air-tight syringe and stored as pellets (in LN₂) prior to use.

1. Cell Lysate Activity Determination.

The cell lysate activity (Table S5, in the unit of nmol H₂/NH₃ per milliliter cell lysate per minute) in both enzymatic proton reduction (H₂ generation) and the dinitrogen-water reduction was determined by a modified method reported by Dean et al, (Refs. 32S7 47).

2. Proton Reduction.

Enzymatic proton reduction was measured in a 25 ml septum sealed vial under a UPC argon atmosphere. One (1) ml from the reaction phase containing 25 mM TES (pH 7.4), 2.5 mM ATP, 5.0 mM MgCb, 30 mM creatine phosphate and 0.125 mg creatine phosphokinase (CPK) was fully degassed, sodium dithionite (solid) was then added to a final concentration of 20 mM. The headspace was charged with argon, and the vial was kept in a 30° C. water bath. The reaction was initiated by injection of 50 μl freshly thawed cell lysate (pellets stored in LN₂) and terminated at 15 min by injecting 0.25 ml 2.5 M H₂SO₄. The headspace gas was analyzed by gas chromatography (SRI 8610C) with a molecular sieves 5 A column and a thermal conductivity detector (TCD) (sampling volume: 0.1 ml). The coefficient between peak area and amount of H2 (nmol) was determined by pure H2.

3. N₂—H₂O Reduction.

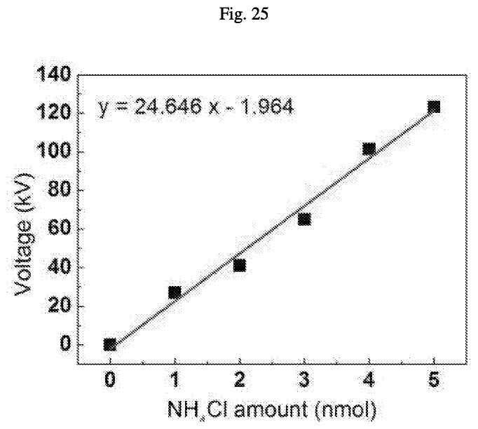

Enzymatic dinitrogen-water reduction was conducted using a method similar to the one used for proton reduction, with the replacement of the headspace gas by UHP N₂ and the buffer by 35 mM HEPES (pH 7.4, to avoid interference in the NH₃ assay). 0.25 ml 0.4 M EDTA (pH 8.0) was injected after 15 min to terminate the reaction. The amount of NH₃ was determined using the o-phthalaldehyde fluorescence method (Refs. 3, 48). 25 μl from the reaction phase was added to 0.5 ml of assay reagent (pH=7.3) containing 20 mM o-phthalaldehyde, 0.2 M sodium phosphate, 5% ethanol and 3.4 mM mercaptoethanol. The mixture was maintained in the dark for at least 30 min and the emission was measured at 472 nm with a 410 nm excitation. NH₃ calibration curves were obtained by using NH₄CI in the same assay. Different concentration of NH4CI was used to obtain the calibration curve (FIG. 25).

B. MoFe Nitrogenase (MFN) Purification.

The MFN protein from A. vinelandii DJ995 bacteria has a 7× histidine tag on the C-terminus of the a-subunit, allowing it to be purified using immobilized metal affinity chromatography (IMAC) (10, 25). The zinc ion was selected as the binding metal due to the use of sodium dithionite (DTT) reducing agent in the buffer. The column (Hitrap IMAC FF 1 ml, GE Healthcare) was charged with zinc (0.1 M ZnSO4/O and equilibrated with fully degassed equivalent buffer (with 2 mM DTT, pH 7.9). The cell lysate was loaded on to the column and washed with equivalent buffer and washing buffer (25 mM Tris-HCl, 0.5 M NaCl, 20 or 0.2? mM imidazole (PMSF) and 2 mM DTT, pH 7.9) to remove non-specific proteins. The His-tagged MFN protein was eluted using the elution buffer (25 mM Tris-HCl, 0.5 M NaCl, 250 mM imidazole and 2 mM DTT, pH 7.9). The elution (dark brown color) was stored as small pellets in LN2, for future use. Protein purity and concentration were determined using sodium dodecyl sulfate-polyacrylamide gel electrophoresis (SDS-PAGE) and Bradford assay, respectively.

C. C. necator Culture.

Wild-type and genetically-engineered C. necator strains^{S1,S8} were utilized for PHB (e.g. H16+pBBR1-PphaC-YFP) plasmid, (pBBRl-yfp) plasmid, etc., ethylene (pBBRl-efe) plasmid, isopropanol (H16_IPAl-10_int) chromosome integration, and 2,3-butanediol (H16_BO2-20_int) chromosome integration. The strains were grown at 30° C., with shaking at 200 rpm, in the minimal salt media (MSM) supplied with 0.2% fructose and 0.2% glycerol.

Strains for methyl-ketone (MK) production was grown in MSM supplied with 2% fructose, and the production of MK was realized via induction with 0.2% L-arabinose after 24 h inoculation (count as t=0).

D. Construction of C. necator Plasmids and Strains.

Plasmids were constructed in the pBBR1-MCS2 backbone. E. coli strains were propagated at 37° C. in lysogeny broth (LB). Where necessary, medium was solidified with 1.5% (wt/vol) agar and supplemented with 50 μg/ml kanamycin. The promoter from the phaC gene in C. necator was used to drive transcription of YFP (control), Ethylene-forming enzyme (efe, from Pseudomonas syringae). Ref S9. Conjugation was performed by transformation of plasmids into the mobilizing strain S-17 E. coli followed by incubation of the transformed cells overnight with C. necator on LB, followed by selection on LB+15 μl gentamicin (to select against S-17 cells) and 300 μl kanamycin. Ref. S10. Transformation of plasmids was verified by colony PCR and/or plasmid extraction and restriction digest validation.

E. Analysis of the Products Generated from C. necator.

For PHB, isopropanol (IPA), and 2,3-butanediol (BDO) production, the culture was sampled (1 ml) at a specific time point for analysis. For methyl (MK) production, after induction with L-arabinose (0.2% w/V), 1 ml culture was sampled for assay. C₂H₄ production was conducted in a septum-sealed vial, with 5 ml liquid phase containing OD600=1.0 cells in MSM media (with 40 mM formic acid). The headspace (25 ml) was sampled at a specific time point.

For PHB analysis (Ref. 33), 1 ml culture was centrifuged, and the pellet (washed once with MSM media) was mixed with 0.4 ml 3:7 HCkMeOH and 0.6 ml 1,2-dichloroethane (DCE) and incubated at 100° C. for two hours with gentle shaking (every 15 min). After cooling to room temperature, 0.3 ml DI water was added with vigorous shaking and the bottom layer (DCE layer) was injected into the GC (1 μl, oven temperature: 140° C.) for analysis.

For IPA and BDO analysis, 1 ml cell suspension was lysed with an ultrasonic probe (3 cycles, 1 min each) followed by centrifuging at 15,000 rpm for 10 min. The supernatant was directly injected (1 μl) into the GC. For IPA, a constant oven temperature (140° C.) was used, whereas programmed temperature ramping (140° C. for 2 min, and 10° C./min ramping to 200° C.) was used for BDO analysis.

For MK analysis (Ref 33), 5 ml cell suspension was mixed with 2 ml hexane followed by vigorous shaking for 30 min. After centrifugation (5,000 rpm, 5 min), the upper hexane layer was collected and concentrated (using N₂ flow) to 100 μl. One (1) μl of the hexane layer was injected for GC analysis (180° C. constant oven temperature).

The generation (accumulation) of the corresponding products, together with the bacteria cell optical density (at 600 nm) are shown in FIG. 16A-H.

TABLE S4
Carrier lifetime and coefficient extracted from a bi-exponential
fit of the open circuit potential (OCP) decay curve.

| CdS@ZnS | A₁ (V) | t₁ (s) | A₂ (V) | t₂ (s) |
|---|---|---|---|---|
| OML | −2.301 | 1482 | −0.158 | 5680 |
| 1 ML | −46.82 | 271.7 | −0.0604 | 4734 |
| 2 ML | −1.190 | 544.0 | −0.204 | 3958 |
| 3 ML | — | — | — | — |

TABLE S5
Cell lysate activity for enzymatic proton and dinitrogen-water reduction.

| | Product | Cell Lysate Activity (nmol/ml CL/min) |
|---|---|---|
| Proton reduction | H₂ | 260.4 |
| Dinitrogen-water | H₂ | 108.7 |
| reduction | NH₃ | 112.5 |

### V. QDs-Enzyme Complex Preparation And Characterization.

A. UV-VIS Determination of QDs-Protein Binding.

Further evidence supporting QD-attachment is provided by the UV-VIS measurements of solutions containing CL and a 500 nM concentration of CZSe3. Samples were incubated at room temperature for 30 min in 35 mM HEPES buffer prior to performing the following experiment.

Initially, strong PL and absorbance of the QDs in FIG. 16A-B is observed with minimal contribution from CL. After centrifuging at 5000 rpm for 5 min, the isolated pellet containing CL proteins is clearly luminescent due to attachment of the water-soluble QDs, which would normally not precipitate from solution at such a mild rpm. The PL and absorption spectra of the supernatant confirm this (FIG. 16C-D), showing that a very small amount of QDs are present in the supernatant (not attached to the CL proteins). Spectra of the redispersed QDs-CL pellet (FIG. 16E-F) shows confirmation that QDs are retained within the pellet by CL.

B. Fourier-Transformed Infrared Spectroscopy (FTIR).

Thermo Nicolet 6700 FTIR instrument was used for infrared spectroscopy measurements, using a germanium attenuated total reflectance (ATR) accessory. QDs-cell lysate, QDs, and cell lysate samples were prepared for the test. Cell lysate (prepared from A. vinelandii DJ995 or C. necator pBBRl-efe) in Tris buffer was desalted (using 10 kDa centrifugal device) and washed with DI water twice to remove interfering Tris components. QDs-cell lysate complex was prepared by mixing the cell lysate with MPA-coated CZSe₃ and incubated at room temperature for 30 min, followed by centrifugation at 15,000 rpm, the resulting pellets were washed twice with DI (deionized) water. No precipitate was observed when cell lysate or MPA-coated QDs suspension (in pH 11 water) was centrifuged at 15,000 rpm up to 30 min. The pellets formed in the mixture are due to the formation of QDs-protein complex. Samples were drop-casted on clean glass slides followed by air-drying.

FTIR was used to show proteins coupled to the QDs. The QDs are able to trap the proteins from the cell lysate, as shown by the similarity of the QDs-CL and CL spectra (FIG. 16G-H), with characteristic C=0 stretching (1655 cm⁻¹), N—H bending (1540 cm⁻¹), and N—H stretching (3250 cm¹, broad due to hydrogen bond formation) indicating the presence of bound proteins (amide). Coupling of the protein to the QDs is further proven by the disappearance of the O—H stretching (3395 cm¹, broad) in the MPA-capped QDs, where the —OH group from the original carboxyl terminated QDs was replaced by the —NH bond through amidation with the amine bond.

C. SDS-PAGE Protein Electrophoresis: Determination of Protein Bound to CdS or ZnS.

To evaluate the selectivity of CdS and ZnS in protein binding, cellular protein trapped by their corresponding particles were analyzed by sodium dodecyl sulfate-polyacrylamide gel electrophoresis (SDS-PAGE).

1. Selective Protein Binding to CdS or ZnS.

CdS and ZnS particles were synthesized using the reaction of 0.1 M Cd²⁺ (CdCl₂) or Zn²⁺ (ZnSO₄) with 0.1 M S²⁺ (Na₂S). The particles were washed with water (twice) and equivalent buffer (twice) and suspended in equivalent buffer. About 10 mg CdS or ZnS were charged with 1 ml cell lysate and incubated at 4° C. for 1 hour. The mixture was then centrifuged and washed with equivalent buffer (0.3 ml, twice) and wash buffer (0.3 ml) and re-suspended in 0.1 ml equivalent buffer.

2. Determination of Protein Bound to CdS or ZnS.

The proteins bound to CdS or ZnS were determined using SDS-PAGE. The CdS and ZnS particles with trapped (bound) proteins were boiled with SDS sample buffer and centrifuged at 10,000 rpm to remove the particles. The samples were loaded on the 12% SDS-PAGE gel, and the electrophoresis was run using constant voltage (200 V), followed by staining with Coomassie G250 to show the protein bands (FIG. 2AB). Cell lysate and purified MoFe nitrogenase were also tested as references.

Selective His-tagged protein binding can be clearly seen with ZnS particles, showing an almost single protein band (FIG. 2AB, lane 5 FIG. S6A, lane 5). Compared to the purified MFN (FIG. 2AB, lane 3), its selectivity in His-tagged MFN is as good as the commercial Zn-IMAC column. On the other hand, CdS showed almost no selectivity in protein binding (FIG. 2AB, lane 4). This test shows that ZnS-coated CdS nanoparticles for selective MoFe nitrogenase binding for photocatalytic H₂ or NH₃ production is preferred.

The A. vinelandii MoFe protein has two alpha (MoFe co-factor) and two beta (P-cluster) subunits, with molecular weights of 55.3 (plus approximately 1 for 7× histidine tag) and 59.5 kDa, respectively.

D. Agarose Gel Electrophoresis (AGE).

Agarose Gel Electrophoresis (AGE) was performed to prove the selective coupling between the ZnS-coated QDs and His-tagged MFN. Different proteins in the cell lysate are separated based on their molecular weight and charge, and the coupling of proteins to the QDs could show a change in the electrophoretic migration pattern. To use visible QDs, we used MPA-capped CdSe@ZnS QDs (CZSe1 and CZSe2 with peak emission at 540 and 570 nm, two monolayer ZnS coating) instead of non-fluorescent CdS@ZnS QDs for fluorescent detection of the QDs. The fresh-thawed cell lysate was mixed with QDs and incubated at room temperature for half an hour, followed by loading on 1% agarose gel. The electrophoresis was performed under constant voltage (200 V) mode for 1 hour. The gel was then gently rinsed with DI water and imaged using Gel Doc EZ imager on a UV tray (Bio-Rad), with excitation at 470 nm and emission at 570 nm, (FIG. 2AC: panels A and C). Protein locations were indicated (FIG. 2AC, panels B and D) by using Coomassie brilliant blue G250 staining as above, and as described herein. Control experiments with one each of cell lysate, QDs-purified MFN mixture, purified MFN, and QDs were also performed on the same gel.

Fluorescence images clearly shows the existence and the position of QDs in the gel (FIG. 2Ac, panel A) The migration of the QDs depends mainly on their size and surface charge, and a larger size QDs (FIG. 2AC, panel C, lane 8) show slower migration compared to the smaller ones (FIG. 2AC, panel C, lane 7). Coupling between the QDs and the purified MFN (formation of QDs-MFN complex), FIG. 2AC, panel C, lanes 5 and 6, resulted in a large lag in electrophoretic migration due to a significant increase in particle (QDs-protein corona) size. We have also shown selectivity in the binding of QDs with a specific protein, by comparing the electrophoretic migration patterns of QDs-cell lysate and QDs-purified MFN in the fluorescence and staining images. Multiple bands in the staining image (right figure, lane 1 to 3) indicate the existence of different proteins (with varied molecular weight) in the cell lysate, while the fluorescence mainly originates from the single band of MFN, as indicated by the same emission position of QDs-cell lysate (lane 2 and 3) and QDs-purified MFN (lane 5 and 6). This proved that the majority of the QDs are bound to the MFN, and this selectivity between ZnS-coated QDs and His-tagged MFN is based on the zinc-histidine affinity. To further verify that the QDs can selectively couple the His-tagged MFN, the fluorescent band was cut for orbitrap mass spectroscopy to identify the proteins. As shown in the attached “Protein Mass Spec.xlsx” file, the two major peptides detected (with highest MS/MS count) is the nitrogenase beta chain and alpha chain, demonstrating that the ZnS-coated QDs preferentially trap the His-tagged proteins.

E. Inductively-Coupled Plasma Mass Spectroscopy (ICP-MS).

ICP-MS was used to determine the level of cadmium to quantify the efficiency and selectivity of QDs-protein coupling. The CZSe2-cell lysate lane (FIG. 2Ac, lane 3) from the agarose gel was utilized, and the MFN band and one non-MFN band was excised from the gel and digested with nitric acid, in a final sample volume of 0.5 ml. Cell lysate lane (lane 1) with corresponding bands were used as control (baseline for the cadmium level). Selectivity of His-tagged MFN attachment to the ZnS-coated QDs was seen in FIG. 2Ad, FIG. 176C, as the cadmium level (ppb) in the MFN band is more than three times the level in the non-MFN band.

F. QDs-MFN Biohybrid for Light-Induced Proton Reduction: CdX:Nitrogenase Biohybrid Photocatalytic Proton Reduction.

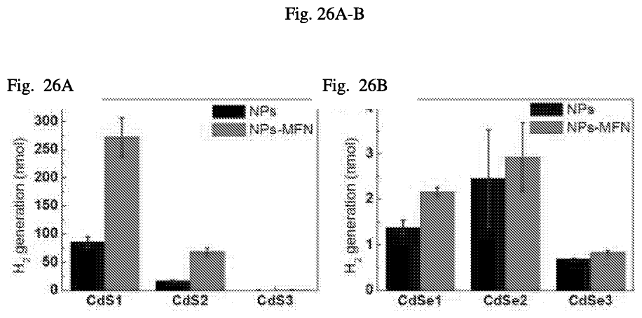

MPA-capped CdS or CdSe QDs were anaerobically mixed with the purified nitrogenase. The mixture was incubated at room temperature for about 10 min and diluted with fully degassed 100 mM L-ascorbic acid (pH=7.4). The mixture (with 200 nM QDs and 66 nM nitrogenase) was anaerobically transferred to several argon-purged 2 ml GC vials (with a small magnetic stirrer, 0.3 ml liquid volume). Light-induced proton reduction was performed by irradiating the system with a 400 nm LED panel at 1.6 mW/cm². Headspace gas was sampled after 30 min irradiation. The net H₂ turnover number (TON) are shown in FIG. 17A and FIG. 26A, for examples. FIG. 26A-B and FIG. 17A.

G. Light-Induced Redox Reaction with QDs-CL Mixture.

Here, the QDs refer to CZSI with nominal 0 to approximately 3 monolayer ZnS shells, synthesized from 3.55 nm CdS QDs, and capped with MPA ligands.

Light-induced proton reduction reaction was taken in a 2 ml vial under stirring, with a total reaction volume of 0.3 ml. The reaction phase contained 200 nM QDs, 100 mM ascorbic acid (pH 7.4) was vacuum-degassed and charged with argon (approximately 1.7 ml headspace). Anaerobically thawed cell lysate (15 μl) was swiftly injected into the vial with an air-tight syringe. The mixture was incubated at 30° C. for 10 min, followed by irradiating using a 400 nm LED panel (with −1.6 mW/cm² at reaction site) for 30 min. The headspace gas was analyzed by gas chromatography (0.1 ml sampling) using the above-mentioned method, and as described herein. FIG. 17B.

The light-induced N₂—H₂O reduction was done in a similar condition, with the replacement of ascorbic acid by 300 mM HEPES and the headspace gas by UHP grade N₂. H₂ and NH₃ were analyzed using the methods mentioned above, and as described herein, (FIG. 17C).

To evaluate how the selective binding of the His-tagged MFN to the ZnS-coated QDs would effect H₂ production, the reaction was performed in media with the addition of 250 mM imidazole and with a higher acidity (pH 5.9). In both cases, the selective binding was interrupted.

The total amount of H₂ produced from the QDs-cell lysate systems (xQDs-CL) was compared to the QDs (xQDs) (x=0-3 indicating the number of ZnS shells) systems. The OQDs-CL shows (FIG. 17D) a minor increase in H₂ production compared to 0NP, this is due to non-selective binding (through electrostatic interactions) of the QDs to both active MoFe nitrogenase and non-active cell components. These two different bindings will either enhance or decrease H₂ production due to the catalytic effects and potential surface reaction site blockage, and hence no obvious improvement in H₂ generation was observed. Due to the difficulty in tunneling electrons through the thick ZnS barrier layer, 3QDs-CL also show a low H₂ yield. However, a significant increase in H₂ production was observed in 1 QDs-CL (by 2.9 folds) and 2QDs-CL (by 1.6 folds) compared to 1QDs and 2QDs, respectively, with the highest rate of H2 production being 3467 nmol/ml CL/h for 2QDs-CL. The improvement in H2 yield could be due to the site-selective His-tagged MFN to the QDs zinc-rich surface binding, which was also confirmed by control experiments, with the addition of imidazole (at a high concentration of 250 mM, pH 7.4) or using a higher acidity (pH 5.9) environment. Such selective binding is prevented by either competitive imidazole-zinc coordination or the protonation of histidine, and as expected, no improvement in H₂ production was seen between xQDs-CL and xQDs ((FIG. 17E-F.

For the light-induced dinitrogen-water reduction with QDs:cell lysate biohybrids, both H₂ and NH₃ were generated (FIG. 17). Similar to the proton reduction, a significant increase of H₂ and NH₃ yield was observed in 1 QDs-CL and 2QDs-CL systems, with maximum H₂ and NH₃ production rates of 1587 and 693 nmol/ml CL/h for 2QDs-CL. No improvement of H₂ generation was observed in 0 QDs-CL and 3QDs-CL. The difference of 1:2 in the ratio of H₂ to NH₃ is probably due to direct proton reduction from the QDs to the biohybrids.

### VI. Interactions Between QDs and the Living Bacteria.

In this section, and as described herein, cellular uptake (elemental analysis and confocal microscopy) and a series of viability tests (cell growth assay, resazurin dye assay, CFU assay) were performed to study the interactions between the QDs (CZSI with nominal 2ML ZnS shell, if not specified) and the bacteria cells (A. vinelandii and C. necator). Nitrogen-free Burk media or photocatalytic media (35 mM HEPES and 5 to approximately 25 mM L-ascorbic acid, pH=7.4) were used for A. vinelandii test, and minimal salt media (MSM) was used for C. necator test.

A. Cellular QDs Uptake Assay.

A. vinelandii DJ995 cells were collected at mid-log phase (OD600 approximately 1.0) and washed twice with ASC5 media (35 mM HEPES, 5 mM L-ascorbic acid, pH=7.4). Mixture with 200 nM MPA, CYS, or CA-coated QDs and OD6oo approximately 1.0 bacteria cells in ASC5 media (total volume 200 μl) were incubated at 30° C. for 30 min. The mixture was then centrifuged at 6,000 rpm and the cell pellets were quickly washed with ASC5 media (0.5 ml, twice) and finally re-suspended in 0.5 ml ASC5 media. The amount of Cd (in ppb) is measured by ICP-MS. The Cd level in the QDs suspension (1, 10, 100, 1000 nM) was also measured. A calibration curve was used to correlate the QDs concentration (in nM) to the Cd level (in ppb).

The QDs uptake (FIG. 2F) is calculated by counting the percentage of QDs (in numbers) consumed by the cells:

QDs uptake (%)=ICP−MS measured QDs concentration×500×100%

### Initial QDs Concentration ×200

B. Laser-Scanning Confocal Microscopy.

A. vinelandii cells were taken from growth media and centrifuged at 4,000 rpm for 2 min, washed twice with 25 mM HEPES buffer at pH 7.4, and resuspended in the same HEPES buffer to an OD of 0.1. MPA-capped CZSe were added to cells in HEPES buffer for a final concentration of 250 nM. The QDs-cell mixture was vortexed briefly and placed in a shaker at 37° C. and 225 rpm for one hour. The mixture was then centrifuged, washed with HEPES buffer three times, resuspended to an OD of 0.1, and deposited into a well-plate treated with poly-L-lysine. Samples were incubated at room temperature for 30 min, after which the wells were rinsed twice with DI water. 0.1 mL 4% paraformaldehyde solution was added to each well and allowed to incubate for 10 min at room temperature. After fixation, wells were rinsed with DI water twice and evacuated of liquid prior to imaging. Samples were imaged using Nikon AIR laser scanning confocal microscope with immersion oil and a 100× oil objective. Z-scan images were taken using a step-size of 0.25 m with an x-y resolution of 200 nm.

Confocal images were recorded (FIG. 18A-L) to provide direct evidence that CZSe3 penetrate and reside within the cells after incubation. Bright emission was seen under TRITC laser, with the general shape corresponding to the profile of the cell, indicating the internalized CZSe3 QDs. 3D single cell rendering with several Z-scan images of the same cell (from the focal plane at the bottom of the cell to the top) demonstrate that the QDs are internalized into A. vinelandii cells instead of aggregating on the cell membrane. Internalization of the other QDs used in this study is feasible, considering their smaller sizes compared to CZSe3 QDs.

FIG. 18A also depicts a large cluster of cells in transmission mode with no excitation. When the same cluster is illuminated with TRITC laser, the internalized CZSe3 show bright emission (FIG. 18B and FIG. 18C), which take on the general shape of the cells within the cluster. Further evidence of QD internalization is demonstrated in the 3D rendering of a single cell (FIG. 18D) where the QDs occupy the full volume of the cell. Several Z-scan images of the same cell are shown in FIG. 18E-L) where FIG. 18E begins with the focal plane at the bottom of the cell and ends at the top (FIG. 18L). These series of images demonstrate that the A. vinelandii cells are capable of internalizing CZSe3. Internalization of the other QDs used in this study is feasible, considering CZSe3 have the largest diameter.

C. Photocatalytic Reaction for Nanoparticle-Cell Lysate (NP-CL) Biohybrid.

Photocatalytic proton reduction reaction was taken in a 2 ml vial under stirring, with a total reaction volume of 0.3 ml. The reaction phase contained 200 nM nanoparticles, 100 mM ascorbic acid (pH 7.4) was vacuum-degassed and charged with argon (−1.7 ml headspace). Anaerobically thawed cell lysate (15 ul) was swiftly injected into the vial with an air-tight syringe. The mixture was incubated at 30° C. for about 5 min, followed by irradiating using a 400 nm LED panel (with about 1.6 mW/cm² at reaction site) for 30 min. The headspace gas was analyzed by gas chromatography (0.1 ml sampling) using the method mentioned above. Reaction media with the addition of 250 mM imidazole and with higher acidity (pH=5.9) were also used.

Photocatalytic N₂ reduction was taken in a similar condition, with the replacement of ascorbic acid by 300 mM HEPES and the headspace gas by UHP grade N₂. H₂ and NH₃ were analyzed using the methods mentioned herein.

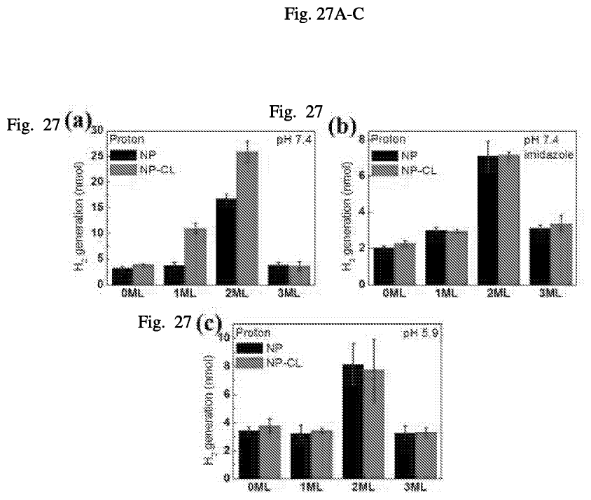

The total amount of hydrogen produced from nanoparticle-cell lysate systems (xNP-CL) was compared with the nanoparticle- (xNP) systems (x=0˜3 indicating the number of ZnS shells. The ONP-CL shows (FIG. 27a) a minor increase of hydrogen production compared to ONP due to non-selective binding of nanoparticles to both active MoFe nitrogenase and non-active cell components through electrostatic interactions. These two different bindings will enhance and decrease hydrogen production due to catalytic effects and surface reaction site blocking, respectively, and hence no obvious improvement of Ff₂ generation. And due to the difficulty of electron tunneling through the thick ZnS barrier layer for electron injection to MoFe nitrogenase, 3NP-CL also shows low hydrogen yield. On the other hand, a significant increase of hydrogen production was observed in 1NP-CL (by 2.9 folds) and 2NP-CL (by 1.6 folds) compared to 1NP and 2NP, respectively. The highest Ff₂ generation rate reaches 3467 nmol/ml CL/h in 2NP-CL. Site-selective binding of His-tagged MoFe nitrogenase on the zinc-rich surface could be one explanation of this high yield. Control experiments with imidazole addition (at a high concentration of 250 mM, pH=7.4) or using higher acidity (pH=5.9) environment were performed. Imidazole could competitively bind to zinc and block the available sites for histidine attachment. In lower pH media, histidine is protonated and not able to coordinate with zinc. And as expected, in both cases no change of hydrogen production (FIG. 27b, FIG. 27c) between xNP-CL and xNP was observed.

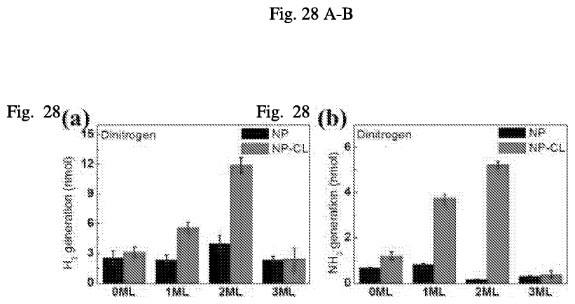

In the case of dinitrogen reduction with MoFe nitrogenase, both H2 and NH₃ were generated (FIG. 28A-B). Similar to proton reduction, a significant increase of H2 and NH₃ yield was observed in 1 NP-CL and 2NP-CL, with maximum hydrogen and ammonia production rate of 1587 and 693 nmol/ml CL/h in 2NP-CL. And no improvement of hydrogen yield was observed in ONP-CL and 3NP-CL. Compared to the enzymatic dinitrogen reduction with MoFe nitrogenase, the deviation of H2 to NH₃ ratio from 1:2 is probably due to direct hydrogen generation from nanoparticles.

D. Cell Growth Curve Measurement.

For the following test using living cells, if not specified, the nanoparticles refer to CdS@ZnS nanoparticles with nominal two-monolayer ZnS shell (CZS). The photocatalytic media refers to 35 mM HEPES buffer (pH=7.4) with 5, 10 or 25 mM L-ascorbic acid (ASC5, ASC10, and ASC25, respectively) as sacrificial hole quencher.

Cell growth measurement was performed in either Burk media or photocatalytic media (PCM), with a variation of QDs (nanoparticle) capping ligands (MPA, CYS, and CA) and concentration (50, 100, 200, 500, 750, and 1000 nM). A. vinelandii DJ995 bacteria grown in nitrogen-free Burk media were harvested at OD₆₀₀ approximately 1.0 (mid-log phase) and washed twice and re-suspended in the Burk media or photocatalytic media. The cell growth curve (by measuring the optical density at 590 nm) was taken in the 96 well microplate (30° C., vigorous shaking) and monitored using a microplate reader (TECAN GENios) controlled by Megellan 7.2 software. Cells treated with QDs in photocatalytic media followed by growing in nitrogen-free Burk media were also tested to evaluate their viability after QDs treatment. The cells were first incubated in photocatalytic media (with QDs) for two hours (either in the dark or under 1.6 mW/cm², 400 nm irradiation), followed by washing and re-suspending in Burk media for the growth. For all these cell growth measurements, the initial cell OD₆₀₀ is 0.1.

The inhibition of cell growth indicates the QDs toxicity, which is strongly ligand-dependent (FIG. 18A-D. While MPA- or CA-capped CZS show significant inhibition of cell growth, no such inhibition was seen with CYS-capped CZS. And a decrease of cell toxicity was observed with ZnS shell, as shown in the decrease of cell growth inhibition with CYS-coated CZS (FIG. 18B) compared to CYS-capped CdS QDs (FIG. 18D). The ZnS shell could possibly prevent the leakage of Cd²⁺ ions from the CdS cores and hence reduce their toxicity.

No cell growth was observed (FIG. 18E-F in the photocatalytic media (non-growing media). The bacteria stay dormant and can (partially) resume growth once re-suspended in Burk media, as seen in FIG. 18G-L.

Cell treatment in the dark revealed the capping ligand-dependent cell viability. While no remarkable cell viability loss was seen with MPA or CYS-capped QDs, a decrease of cell viability was observed with CA-coated QDs at higher concentration (small decrease at 500 nM and a complete cease of cell growth at 750 or 1000 nM). This indicates that dark cytotoxicity is very low for MPA or CYS-capped QDs, where the nanoparticle surface is negatively charged or has zwitterion characters, respectively. On the other hand, CA-capped QDs with positive surface non-selectively bind to all cell components, showing high cell toxicity.

The photo-toxicity of these QDs is similar to their dark toxicity. While CYS-capped QDs show no loss of cell viability up to 1000 nM, some cell viability loss was observed with a high concentration (750 and 1000 nM) MPA-capped QDs treatment. And almost complete ceasing of cell growth in higher concentration CA-capped QDs demonstrates their high toxicity.

D. Resazurin Dye Cell Viability Assay.

Cell viability assay was performed in a 96 well microplate using resazurin dye as an indicator. The bacteria cells (OD600 approximately 1.0) were for two hours treated QDs in the dark or under light (1.6 mW/cm², 400 nm irradiation) irradiation, followed by washing (twice) and re-suspended in the nitrogen-free Burk media (OD600=1.0), as mentioned in the cell growth curve measurements. Resazurin was added to a final concentration of 0.1 mg/ml, and the fluorescence (excited at 485 nm) was measured at 620 nm using the microplate reader.

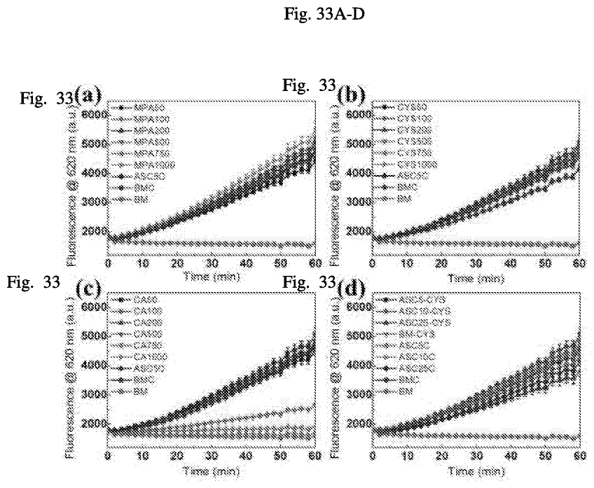

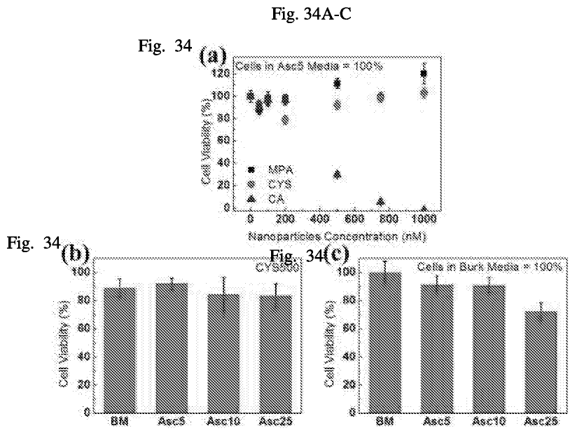

As shown in FIG. 20A, FIG. 33 and FIG. 34A-C, no dark toxicity was seen with MPA and CYS-capped QDs up to 1000 nM.

For CA-capped QDs, cell toxicity was initially seen at 500 nM, and complete loss of cell viability was observed with higher concentrations (750 and 1000 nM). Compared with cell treatment in different media charged with or without CYS-coated nanoparticles (500 nM), a very small decrease of cell viability is shown. Furthermore, cell treatment (without nanoparticles) in different media shows no statistical loss of cell viability with ASC5 and ASC10 compared to cells without treatment (directly growth in Burk media). With L-ascorbic acid at higher concentration (25 mM), a small inhibition effect was observed.

Conclusions obtained from cells treated with QDs (nanoparticles) under light irradiation (FIG. 20D-F) (FIG. 21D-F) are similar to the correspondent cell growth curve measurement (FIG. 19A-L), where CYS-capped QDs show non-toxic characteristics and MPA-coated QDs show some toxicity at high concentration (750 and 1000 nM). For cell treatment with CA-capped QDs, significant loss of cell viability was seen even at low concentration. And compared to resazurin cell viability test with cell treatment in dark, significant decrease of cell viability starts even at low concentration CA-coated nanoparticles, while cell viability is not completely lost even at high nanoparticle concentration, as also seen in the cell growth measurement. Another interesting phenomenon is the cell treatment in different media with or without nanoparticles. While in dark condition, no different of cell viability is seen with media variation, an increase of cell viability in light irradiation condition was observed with increase L-ascorbic acid concentration. This can be explained by L-ascorbic acid as a better sacrificial agent (quencher) for holes compared to sucrose in Burk media. Under 400 nm light irradiation, the photo-generated holes, which are highly cytotoxic, can be easily quenched by L-ascorbic acid and such effect is facilitated with increasing L-ascorbic concentration. Therefore, higher cell viability was observed in ASC25 media.

E. Colony forming unit (CFU) assay.

CFU assay was also used for evaluating the cell viability. Cell cultures were collected at OD600=1.0 from nitrogen-free Burk media and washed twice with ASC5 media. Mixtures with OD600=1.0 bacteria cell, 500 nM MPA, CYS or CA-capped QDs in ASC5 media were incubated at 30° C. for 2 hours in the dark, followed by washing (twice) and re-suspending in the same amount of nitrogen-free Burk media. 10 ul of the suspension with different cell OD600s (1, 10⁻², 10⁻⁴, 10⁻⁶, 10⁻⁸) was inoculated on the B-plate (nitrogen-free Burk media with agar, in a squared petri dish) and incubated under 30° C. The CFU was counted by naked eyes (FIG. 2G). CFU from OD6oo=10⁻⁴ suspension is presented in FIG. 2G, with the calculated cell viability using cells treated with no nanoparticles in ASC5 media as 100%.

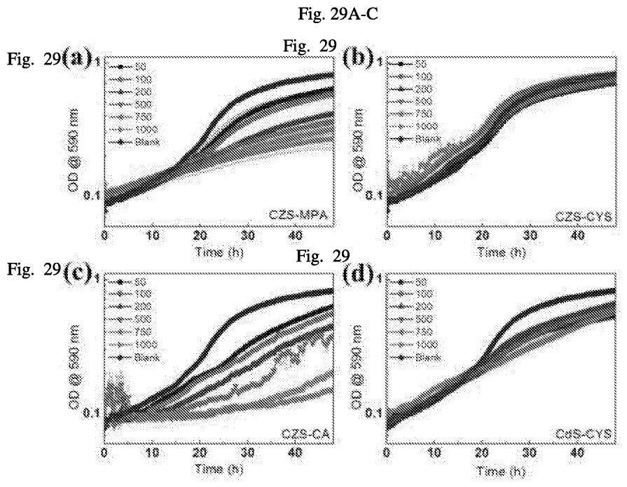

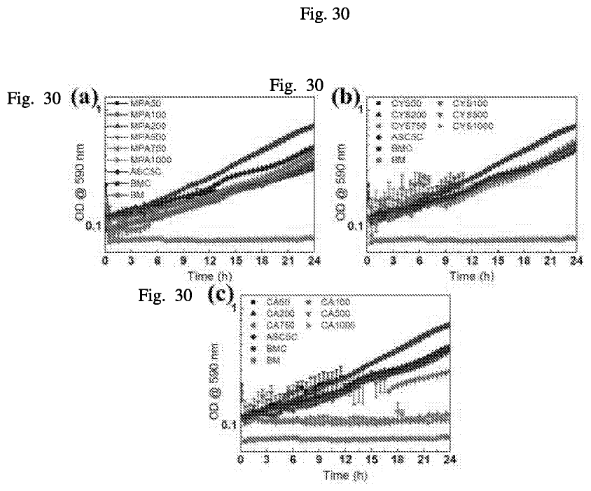

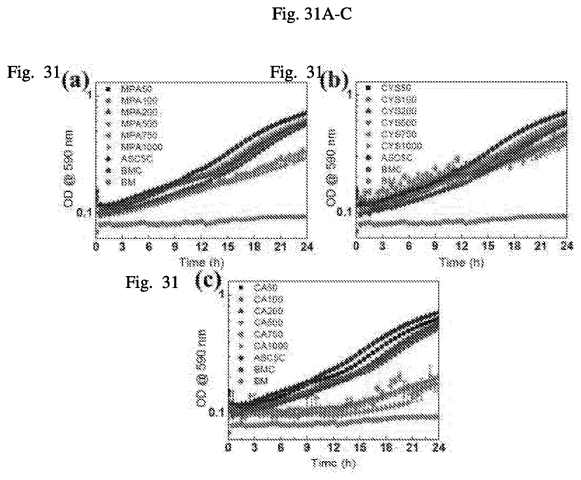

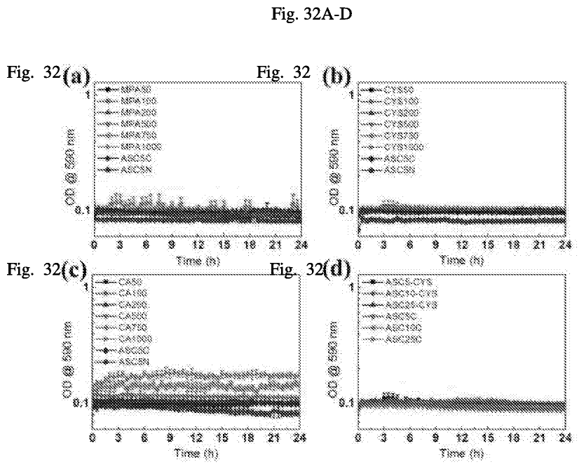

Similar to the cell growth and resazurin cell viability test mentioned herein (FIG. 29-FIG. 35), with MPA and CYS-coated nanoparticles (500 nM) treatment, no decrease of CFU is seen compared to treatment in the same media (ASC5) without nanoparticles. And the highly toxic CA-coated nanoparticles render the CFU to a very low value, showing about 5% cell viability compared to no nanoparticle treatment.

F. In-Vivo Photocatalytic Ammonia and Hydrogen Generation Test.

In vivo photocatalytic reactions were conducted in either 96 well microplates or small test tubes and tested either in the air or pure dinitrogen atmosphere. The mixtures basically contain Azotobacter vinelandii DJ995 cells, nanoparticles, and photocatalytic media and were incubated at 30° C. for 30 min and 150 mixture was added to the wells and a LED panel with 400 nm emission was used to irradiate the system through the cover, in a top-down mode. Ammonia production was determined using fluorescence assay described above, with the same mixture without irradiation (dark) as a baseline. To optimize the condition for ammonia yield, variations of cell optical density (ODeoo), capping ligands of the nanoparticles, nanoparticle concentration, and irradiation intensity were used.

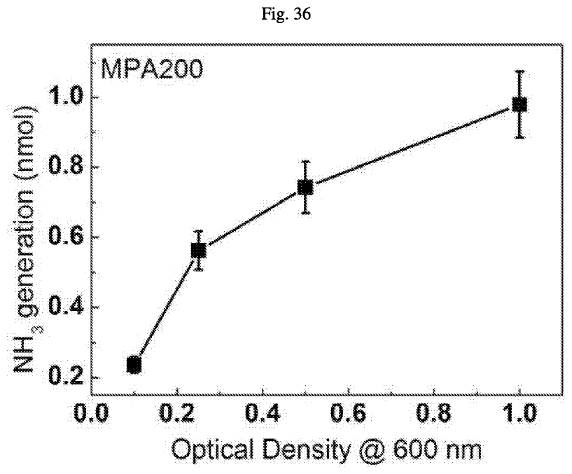

First, 200 nM MPA-coated nanoparticles were used in ASC5 (5 mM L-ascorbic acid, 35 mM HEPES, pH 7.4) and irradiated with 400 nm light at 1.6 mW/cm² for 1 hour. The Azotobacter vinelandii DJ995 culture from the Burk media was centrifuged at 6000 rpm and washed twice with ASC5. The cells were added to the above suspension with final OD600 from 0.1 to 1.0. The net ammonia production is shown in FIG. 36.

The ammonia production increases with cell optical density, but not linearly. As from Azotobacter vinelandii DJ995, OD₆oo=1.0 is at the mid-log phase of its growth and cells will start lysing at higher density. Therefore, we will use OD₆oo=1.0 for our following optimization.

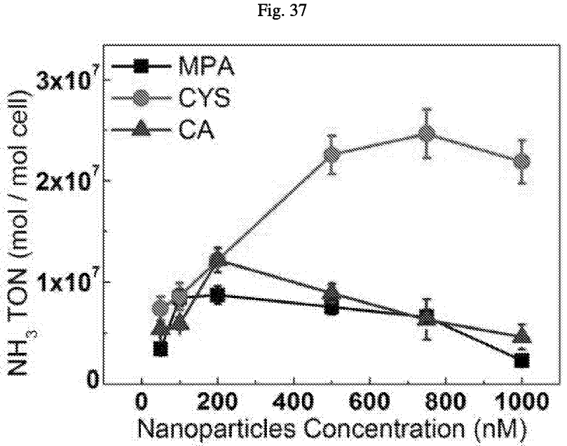

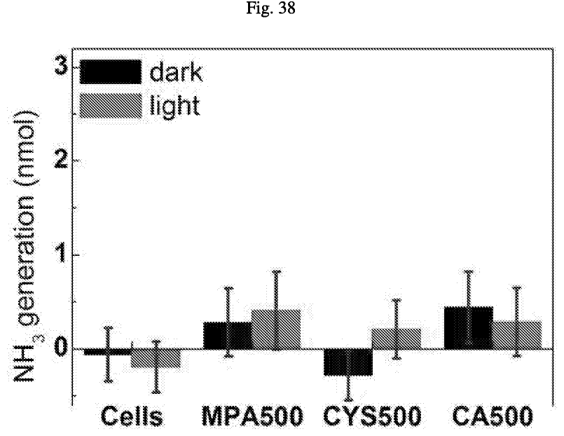

With fixed cell optical density (OD₆oo=1.0), the nanoparticles with different capping ligands (MPA, CYS, CA) and concentrations were used. The ammonia generation is presented in FIG. 37. For MPA and CA-coated nanoparticles, the ammonia yield increases with nanoparticle concentration and have a peak value when 200 nM nanoparticles were used. The yield then drops down with higher nanoparticle concentration. As for CYS-coated nanoparticles, ammonia production levels off at 500 nM nanoparticle concentration, with no further increase or decrease at higher concentration. This concentration and capping ligand-dependent ammonia yield can be related to varieties of factors, including the cell viability and nanoparticle uptake. To ensure high-efficiency photoelectron transfer from nanoparticles to MoFe nitrogenase, higher nanoparticle uptake is required to have more nanoparticles specifically bind to the active enzyme. Meanwhile, the cells should also be at the living condition, where oxygen in the air is consumed without diffusing into the reactive center to deactivate the oxygen-sensitive nitrogenase. With the cell viability measurement (FIG. 31, FIG. 34A-C) with cells treated in photocatalytic media under irradiation, the decrease of ammonia yield at high concentration (MPA and CA-coated nanoparticles) is due to partial loss of cell viability. Though cells have very high uptake for positively charged (CA-coated) nanoparticles, high nanoparticle toxicity at an even low concentration (50 nM) is the main reason for low ammonia yield. Decent uptake and non-toxic character of CYS-coated nanoparticle ensure high ammonia yield. While at lower nanoparticle concentration (50-500 nM) where MPA and CYS-coated nanoparticles show minor loss of cell viability, higher ammonia yield with CYS-coated nanoparticles is mainly due to higher nanoparticle uptake. No further increase of ammonia yield starts beyond 500 nM with CYS-coated nanoparticles could be limited by the amount of bacteria cells.

Control experiments with the removal of some components (cells or nanoparticles) from the mixture were also taken. As shown in FIG. 38, no difference of ammonia production between dark and light. Therefore, we ruled out the possibility of ammonia generation from the bacteria cells or nanoparticles.

Irradiation intensity-dependent ammonia yield is also measured, with 500 nM CYS-coated nanoparticles and ODeoo⁼1-0 bacteria cells. Light intensity at reaction site from 0.16 to 2.42 mW/cm² was used in this assay. With low irradiation intensity, the ammonia yield is low (FIG. 38) due to the limit of photo-induced electrons produced from nanoparticles. However, at high-intensity irradiation, there is a small decrease of ammonia production and this could probably be related to decreasing of cell viability under strong near-UV light irradiation. In the other tests, optimal light intensity (1.6 mW/cm²) was used.

With fixed bacteria, cell optical density (OD600=1.0) and nanoparticle concentration (200 and 500 nM), photocatalytic dinitrogen reduction was taken in the air or pure dinitrogen atmosphere. A small test tube with 150 1 mixture was sealed with a septum and for replacing air with pure dinitrogen gas, the headspace air was vacuumed and recharged with UHP grade N2 using a syringe needle connected to the Schlenk line. The vacuum degassing and N2 recharging were repeated for three cycles to ensure low 02 level in the reaction system. As shown in FIG. 11a, b, no difference of ammonia production was observed between microplate assay and test tube assay in the air, showing no dependence on ammonia production with the experimental setup. No change of ammonia yield was seen in the air or pure dinitrogen when MPA and CYS-coated nanoparticles at 200 and 500 nM were used, which indicates that nitrogen source (dinitrogen in the air or pure dinitrogen) is not a limiting factor. However, with CA-coated nanoparticles, ammonia production increases by almost one fold. This could be explained by the protection of the oxygen-sensitive nitrogenase under an inert atmosphere. Low cell viability in media with CA-coated nanoparticles could render the nitrogenase vulnerable to oxygen toxification as described previously. N₂ protection could be the main reason in higher ammonia production and this also reflects the importance of cell viability in in-vivo light-driven air-water reduction.

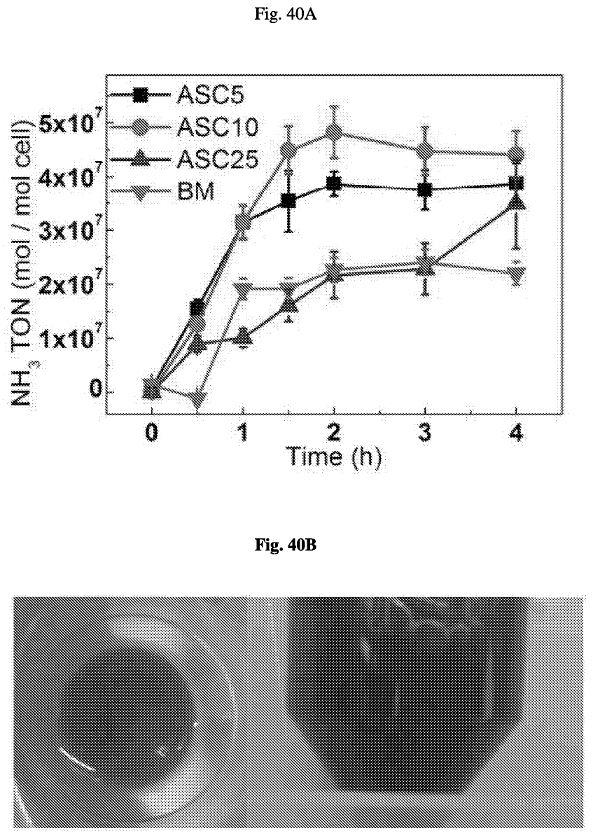

With the above optimization, time-dependent ammonia production was measured using photocatalytic ASC5, ASC10, ASC25 (35 mM HEPES with 5, 10, 25 mM L-ascorbic acid, pH=7.4) and Burk media. The reaction mixture was scaled up from 150 1 to 1 ml to allow multiple sampling. The photocatalytic test was taken in a small test tube covered with aluminum foil and magnetically stirred to ensure enough air supply. 25 microliter reaction phase was sampled at certain time point for ammonia assay. As shown in FIG. 40A, net ammonia generation increases with time and levels off at about 1.5 hours for photocatalytic reaction in ASC5 and ASC10 media. Ammonia yield is lower with ASC25 media but doesn't show saturation up to 4 hours. Unlimited sacrificial agent (L-ascorbic acid) supply could be one explanation and lower cell viability in high concentration (25 mM) L-ascorbic acid can be the reason of lower ammonia production. Furthermore, total ammonia production in Burk media is half compared to ASC5 or ASC10. As from the cell growth curve (FIG. 29-FIG. 32), cells keep growing in Burk media but stay dormant in photocatalytic media. The consumption of generated ammonia could be the main reason of lower ammonia production. This phenomenon was also reported by Harwood et. al., where methane production is much higher in photo synthetic bacteria R. palustris (light-driven CO2 reduction by nitrogenase) with non-growing cells compared to growing cells.

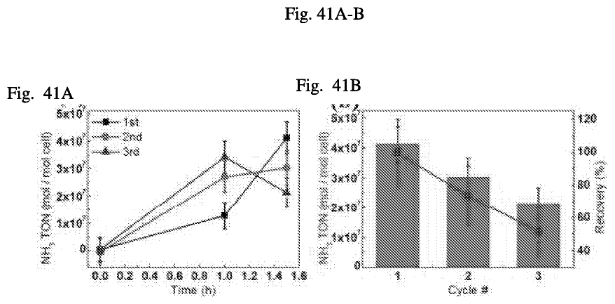

The saturation of ammonia production could be due to depletion of the reducing agent (L-ascorbic acid or sucrose in photocatalytic or Burk media) or an increase of ammonia (inhibitor for MoFe nitrogenase) level in the reaction phase. To prove this assumption, ammonia was removed by separation the cells with centrifugation and replace the reaction phase with new media with nanoparticles every 1.5 hours. As shown from FIG. 41A, ammonia production (partially) resumed with new media, though with decreased yield, as shown in the recovery (FIG. 41b) of ammonia TON from 100% to −75% and −50% in the second and third cycle, respectively. The decrease of recovery could be caused by the loss of cells or cell viability during the long period photocatalytic reaction or with repeated centrifugation and washing.

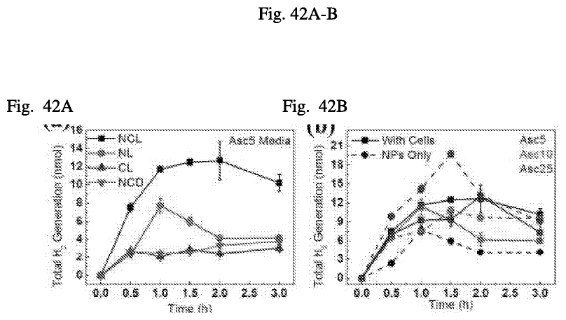

We tested the cell-nanoparticle system in photocatalytic hydrogen production in the air atmosphere. The photocatalytic reaction (1 ml total volume) was performed in a small test tube as described above, with a rubber septum to retain the gas phase used for hydrogen quantification with gas chromatography. Headspace gas (total volume: 7 ml) was sampled at the certain time point and 0.1 ml gas was injected for hydrogen detection. Reaction phase contains 500 nM CYS-coated nanoparticles and OD6oo⁼LO bacteria cells in photocatalytic media (ASC5, ASC10, and ASC25). The result is presented in FIG. 42. Saturation of hydrogen production (FIG. 42a) is seen at 1.5 hours, similar to the case of ammonia production. Control experiments with complete mixture (cells with nanoparticles in ASC5 media) kept in dark and with the removal of nanoparticles do not show detectable H₂ production. And with nanoparticles alone, the H₂ yield is low in ASC5 media. However, with increasing L-ascorbic acid in the media (FIG. 42b), hydrogen production with nanoparticles increases and surpasses the yield with nanoparticle-cell mixture. An increase of hydrogen production with nanoparticles is due to higher quenching rate of photogenerated holes with higher concentration L-ascorbic acid. Due to low nanoparticle uptake, most of the nanoparticles will favor direct charge injection to water for hydrogen production and interaction between non-uptaken nanoparticles will, on the other hand, hamper the hydrogen generation, as shown in lower H₂ yield of nanoparticle-cell compared to the nanoparticle-system in ASC25 media.

The turnover number of the ASC5-CYS500 system for ammonia and hydrogen production was calculated, taking the number of cells (4.5×10⁸/ml×1 ml=4.5×10⁸ at ODgoo⁼1.0) into account. The result is presented in FIG. 11d. Turnover frequency within 1 hour (linear accumulation of ammonia and hydrogen) was calculated to be 8.73×10³ s⁻¹ and 4.35×10³ s⁻¹ for ammonia and hydrogen generation, respectively.

### VII. Formation of QDs-Living Bacteria Nano-Biohybrid (Nanorgs) for Light-Induced Air-CO₂—Water to Fuel Production.

A. Experiment and Control Groups.

The QDs-living bacteria nano-biohybrids were formed by mixing the bacteria cells with QDs in the suitable media followed by incubating at room temperature for about half an hour. Either open-air or septum-sealed reactors were used for photocatalytic air-water or CO₂-water reduction tests. Typically, air-water reduction (NH₃ production) using A. vinelandii was performed in either a 96 well microplate (150 ul total reaction volume, for end-point assay) or a small test tube (1 ml total reaction volume, for kinetics study). CO₂-water reduction using A. vinelandii or C. necator was performed in either a 2 ml GC vial (0.3 ml total reaction volume, for end-point assay) or 30 ml septum-sealed vial (5 ml total reaction volume, for kinetics study). Experiment group for light-induced air-water reduction includes bacteria cells (OD600=1.0), 500 nM CZS QDs (CYS-capped), photocatalytic media (HEPES+L-ascorbic acid), air atmosphere, 1.6 mW/cm² 400 nm LED irradiation. For CO₂-water reduction, the headspace was purged with CO2 for 15 min. Gentle agitation was applied to facilitate gas-liquid phase contact. Control groups with the removal of one or more components were used to prove the product generation from light-activated nanorgs instead of from media or contamination.

1. Plasmid Construction.

Primers were designed to amplify the efe gene with flanking Ndel and BamHI sites at the 5′ and 3′ ends respectively. The efe gene from Pseudomonas syringae pv. phaseolicola was synthesized by Eurofins (e.g. Eurofins Genomics LLC, Louisville Ky. 40299, USA) and was used as a template for PCR amplification utilizing exemplary primers EFEPF ′5 TTT CCC CAT ATG ATG ACC AAC CTA CAG ACT TT 3′ and EFEPR 5′ GGG AAA GGA TCC TCA TGA GCC TGT CGC GCG GG 3′. Both the efeP and YFP plasmids were constructed in the pBBR1-MCS2 backbone. The promoter from the phaC gene in C. necator was used to drive transcription of YFP (control), and the ethylene-forming enzyme (efe, from Pseudomonas syringae). The pBBRl vector was digested with Ndel and BamHI and the efe gene was ligated in to the vector downstream of the Pphac promoter, resulting in the construction of pBBRl-efe. Transformant colonies were selected on Kanamycin (50 mg/ml). Growing transformants were screened via colony PCR utilizing EFEPF and EFEPR Transformants were screened via colony PCR utilizing EFEPF and EFEPR. Sequences, plasmids, transformants, etc., were verified by Sanger sequencing (Source Bioscience). Expression of EFE in C. necator was confirmed via Western blots with EFE antibody (Thermofisher).

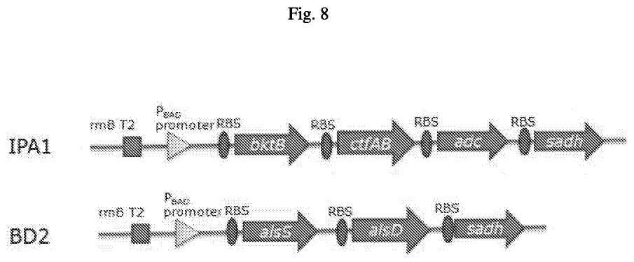

The IPA and 2,3-BDO strains were constructed as shown in FIG. 8, and integrated into the genome of C. necator.

2. Integrated Operons of IPA and 2,3-BDO.

In one embodiment, genes were codon optimized for expression in C. necator H16 (e.g. strain). In one embodiment, bktB(β-ketothiolase, H16_A1445) is native; ctfAB(Succinyl-CoA transferase, AJ000086) from H. pylori, adc(acetoacetate decarboxylase, CA_P0165) from C. acetobutylicum, sadh(secondary alcohol dehydrogenase, AAA23199.2) from C. beijerinckii, alsS(acetolactate synsthase, BSU36010) and alsD(acetolactate decarboxylase, BSU36000) from B. subtilis. pBAD promoter with araC gene was obtained from pCM291rfp plasmid (1) The operons were integrated by a two step homologous recombination using sacB as the selection marker as described previously (2, S11) replacing the native PHB operon (phaCAB).

Primers used for plasmid construction are shown in Table S6. Genes were assembled using the USER assembly method, with the USER cloning kit (New England Biolabs, NEB).

Briefly, a suicide vector pL03(J) (Ref 3) was used for gene deletion and integration. 700 bp upstream of phaC and downstream sequence of phaBl genes were used as homologous regions. The IPA1 and BD2 operons were cloned in between the homologous arms. Alternatively, operons starting from the rrnB T2 terminator were cloned in between the homologous arms.

Suicide vectors, including those described; designed and constructed herein, were transformed via conjugation using E. coli S-17 cells carrying the desired vector and C. necator strains using the protocol described herein and in (Reference (Ref.) No. 2).

Conjugation was performed by transformation of the IPA1 and BD2 suicide plasmids into the mobilizing strain S-17 E. coli followed by incubation of the transformed cells overnight with C. necator on LB, followed by selection on LB+15 μl gentamicin (to select against S-17 cells) and 12.5 μg/ml tetracycline. In some embodiments, transconjugants were selected on minimal media plates with 0.4% fructose and appropriate antibiotics.

A second recombination step was carried out by inoculating single colonies from the first cross-over into low salt LB medium with 15% sucrose without antibiotics overnight. Sucrose resistant colonies were plated onto low salt LB agar plates with 15% sucrose and then single colonies were selected for further screening. Colonies which did not exhibit antibiotic resistance were selected and successful integration of the IPA1 and BD2 operons was confirmed via PCR, utilizing primers flanking upstream and downstream of the homologous sequences. In some embodiments, colonies which did not exhibit antibiotic resistance were selected and successful integration or gene deletions were analyzed using PCR with primers flanking upstream and downstream of the chosen homologous sequences.

Integration of both the IPA1 and BD2 operons and subsequent deletion of the PHB1 operon was confirmed using Sanger sequencing (Eurofins genomics GmbH). Integration or gene deletions were also confirmed using Sanger sequencing (Eurofins genomics GmbH).

For production of the methyl ketones, the plasmid pJM20 was conjugated into C. necator as detailed in Muller et. al., (Ref 33, and as described herein).

TABLE 2
Primers used for plasmid construction.

| | Sequence |
|---|---|
| Primer | (5′->3′) |
| U-ara-F | 1. gggaaagilaacgtt |
| | atgacaacttgacgg |
| | ctac |
| U-ara-R | 2. atatctccUtcttaa |
| | aagatcttttgaatt |
| | ccc |
| U-alsS-F | 3. aggagataUacatat |
| | gaccaaggccaccaa |
| | ggaacag |
| U-alsS-R | 4. acttaacilagcgta |
| | cgtcacagcgccttg |
| | gtcttcatcagc |
| U-alsD-F | 5. agttaagUataagaa |
| | ggagatataacatga |
| | agcgcgagtcgaaca |
| | tccag |
| U-alsD-R | 6. atggttgUcctcctt |
| | tctcgagtcattccg |
| | gcgagccctcg |
| U-sADH-F | 7. acaaccaUgaaggga |
| | tcgccatgctg |
| U-sADH-R | 8. ggagacaUcctaggt |
| | cacaggatcaccacg |
| | gccttg |
| phaC-up-F | 9. acgcgccgatgaaca |
| | ggtc |
| phaB-dn-R | 10. tgctcatcatgccct |
| | gcatcatcg |
| phaC-up-sacl-F | 11. ttattgagctcacgc |
| | cggtcgcttctactc |
| | ctatc |
| phaB-dn-pacl-R | 12. attatattaattaat |
| | cgatgtagttgctca |
| | tcatgccctg |
| phaCB-ov-spel-F | 13. acggcagagagacaa |
| | tcaaatcactagtcc |
| | taggcctgccggcct |
| | ggttcaaccag |
| phaCB-ov-spel-R | 14. ctggttgaaccaggc |
| | cggcaggcctaggac |
| | tagtgatttgattgt |
| | ctctctgccgt |
| U-bktB-F | 15. aggagataUacatat |
| | gacccgtgaagttgt |
| | tgttg |
| U-bktB-R | 16. aacttctcctUtacg |
| | tacgttagatacgtt |
| | caaaaattgctgcaa |
| | tacc |
| U-ctfAB-F | 17. aaggagaagtUacca |
| | tgaacaaggtgatca |
| | cggacc |
| U-ctfAB-R | 18. acttaactagcUcga |
| | gtcacagatgcacct |
| | cgaactcg |
| U-adc-F | 19. agctagttaagUata |
| | agaaggagatataac |
| | atgctgaaggacgag |
| | gtgatc |
| U-adc-R | 20. atggttgUcctcctt |
| | tggatcctcacttca |
| | gatagtcgtagatca |
| | cttcgg |
| U-ctfAB-F | 21. aaggagaagtUacca |
| | tgaacaaggtgatca |
| | cggacc |
| U-ctfAB-R | 22. acttaactagcUcga |
| | gtcacagatgcacct |
| | cgaactcg |
| U-adc-F | 23. agctagttaagUata |
| | agaaggagatataac |
| | atgctgaaggacgag |
| | gtgatc |
| U-adc-R | 24. atggttgUcctcctt |
| | tggatcctcacttca |
| | gatagtcgtagatca |
| | cttcgg |

3. Transformation of C. necator.

In preferred embodiments, plasmids were transformed into C. necator by electroporation. However, it is not meant to limit the method of transformation. As one example, both the pBBR1 efep and pBBR1 YFP plasmids were transformed into C. necator by electroporation. Cells were made competent by growing a 10 ml overnight culture in SOB (Hannahan's broth) media in a Falcon™ tube at 30° C. at 200 rpm with 10 μg/ml gentamycin. The overnight culture was then used to inoculate a 50 ml culture to an OD₆₀₀ of 0.05 with 10 μg/ml gentamycin in SOB media and incubated at 30° C. at 200 rpm until they reached an OD₆₀₀ of 0.3-0.4. Cells were then washed three times with 10 ml ice cold 1 mM HEPES at 4° C. Cells were pelleted by centrifugation at 8000 RPM for 5 minutes at 4° C. The cells were resuspended in 200 ul of 1 mM HEPES. 100-500 ng of pBBRl-efe was added to 100 μl of competent cells in a pre-chilled electroporation cuvette and left on ice for 5 minutes. Electroporation was performed at 2.5 kV, 200Q and 25 uF by a Bio-Rad gene pulser. After electroporation, 0.9 ml SOC media was added to the cells and the cells were transferred to an eppendorf tube and incubated at 30° C. at 200 rpm for 2 hours for outgrowth. After 2 hours of outgrowth dilutions of cells were plated onto LB agar plates with the appropriate antibiotics (300 μg/ml kanamycin in the case of pBBRl-efe) and incubated at 30° C. Transformants appear after 48 hours and were confirmed by amplification of the efe gene by colony PCR.

4. Ethylene Measurements to Confirm Productivity in pBBR1 Efe.

The pBBR1 efe strain was tested for ethylene production using gas chromatography (GC). A single colony was used to inoculate liquid FGN medium, supplemented with 300 μg/mL kanamycin and the cultures was grown for 24 hrs at 30° C. Cultures were then diluted to an OD600 nm of 0.08 in 10 mL fresh FGN medium supplemented with kanamycin (300 ug/ml). 3 mL aliquots were grown overnight in triplicate in 10 mL rubber-capped GC serum bottles at 30° C., 200 rpm. 2 mL of the headspace was collected with a gas syringe after 4, 8, 12, 24, 48 and 72 hrs and analyzed using a Trace™ 1300 gas chromatograph (Thermo Scientific™) under the following conditions: column size: 0.53 mm×40 mm; solid phase: Porapak N column; column temperature: 60° C.; carrier gas: helium and detector: TCD. OD values were determined using a spectrophotometer set at the wavelength λ=600 nm. Ethylene production peaked at 24 hours at 300 nmol/OD₆₀₀/mL ethylene. No ethylene was detected in the control strain pBBR1YFP.

5. Analytical Methods to Confirm Productivity in C. necator IPA1 and BD2.

The C. necator IPA-1 and BD2 strains were both cultivated in minimal media with a C:N (carbon to nitrogen) of 60 (mol C/mol N), utilising fructose as the sole carbon source to induce the stringent response in 50 ml shake flasks at 200 rpm, 30° C. L-Arabinose was added at a final concentration of 1 g/L (0.1% w/v) to the cultures for IPA-1 and BD2 gene induction. Supernatants from the cultivations were obtained by centrifuging the culture samples for 5 min at 13000 rpm. R,R-2,3-BDO and isopropanol were analysed using HPLC with Aminex HPX-87H column (Bio-Rad, Hercules, Calif.), 5 mM H₂SO₄ as mobile phase, equipped with UV and RI detectors. The flow rate of the mobile phase was 0.5 mL/min with a column temperature of 50° C. Quantifications were performed from the standard curves obtained using standards purchased from Sigma Aldrich. Biomass growth was quantified based on optical density measurement at 600 nm using a spectrophotometer. R, R-2, 3-BDO was produced at 0.045 g/L in the BD2 strain and isopropanol was produced at 0.11 g/L in the IPA-1 strain. No R, R-2, 3-BDO and isopropanol could be detected in the control strains.

6. Dark Vs. Light Test.

Control tests were performed with the removal of light irradiation. The lack of any detectable levels of NH₃, H₂, HCOOH, PHB, C₂H₄, IPA, or BDO demonstrates that these products were not generated from the natural metabolism of the bacteria. It also rules out the possibility of any organic compounds (HEPES, L-ascorbic acid, etc.) acting as a source for product generation.

7. Photocatalytic Test with Bacteria Cells or QDs.

Control tests carried out with either no QDs or no bacteria cells showed negligible (FIG. 21A, 21B) amounts of NH₃ or C₂H₄ production.

8. Photocatalytic Test in an Argon Atmosphere.

To further clarify the origin of the nitrogen and carbon in the NH₃ and C₂H₄ products, the same tests were performed in an argon atmosphere instead of air or CO₂. The cell cultures (A. vinelandii or C. necator) were bubbled with argon for 1-2 hours to remove any remaining intracellular nitrogen or CO₂ gas. The collected cells were washed twice and suspended in argon-purged reaction media. The photocatalytic reactions were carried out in the same condition in a septum-sealed vial with argon headspace. No NH₃ or C₂H₄ was detected up to 3 hours and 48 hours, respectively (FIG. 4C, FIG. 21C, 21D). This further proves that the nitrogen and carbon in the generated NH₃ or C₂H₄ is limited to coming from air and CO₂ instead of from the reaction media or other contaminants.

9. Photocatalytic Test with the Microparticles.

To prove our assumption that the photocatalytic air-water and CO₂-water reduction reaction was performed intracellularly, we compared the parallel tests using the sub-5 nm QDs (q-CZS and q-CZSe, as used in the experiment groups) and their bulk counterpart (b-CZS and b-CZSe, acting as the control groups) for both NH₃ and C₂H₄ generation. Bulk CdS was synthesized using chemical precipitation method with 0.1 M CdCl₂ and 0.1 M Na₂S solution. Similarly, bulk CdSe was synthesized with CdCl₂ and Na₂SSeO₃ (by dissolving Se powder in hot Na₂S₂O₃ solution). The b-CZS and b-CZSe were synthesized by coating two monolayer ZnS on the bulk CdS or CdSe with SILAR. Photocatalytic tests were done with replacement of 500 nM QDs with 0.2 mg/ml b-CZS or b-CZSe. No or negligible production of NH₃ or C₂H₄ indicates the necessity of intracellular coupling of QDs with related enzymes for catalytic fuel generation.

10. Photocatalytic Test with the Addition of 2,4-dinitrophenol (DNP).

To prove the direct electron transfer from the QDs to MFN (in A. vinelandii) or hydrogenase (in C. necator) to initiate the air-water or CO₂-water reduction reaction, instead of an indirect, ATP-dependent NADPH-mediated process, control experiments with the addition of ionosphere (DNP) at different concentration were conducted (S12, 34). Ionophores can disrupt the cell membrane potential and inhibit the formation of ATP, thus blocking the NH₃ or C₂H₄ generation under natural production conditions (with consumption of sugar). We have observed (FIG. 4d, FIG. 21E, F) continuous NH₃ and C₂H₄ generation even with a high concentration (0.5 mM) of 2,4-DNP, which demonstrates the direct electron injection-driven production.

11. Photocatalytic Tests with Different Bacteria Strains.

To further prove photocatalytic product generation, we compared different strains of A. vinelandii and C. necator, where the control groups have different metabolic pathways resulting in much lower or no desired product generation. For NH₃ production, A. vinelandii DJ1003 strain (same as DJ995 but also contains an insertion and deletion mutation within nifB) was used as a control, the strain produces apo-nitrogenase (lack of MoFe cofactor) with a low or no nitrogen fixation capability (urea as a nitrogen source is hence required in the culture). The same test was conducted within 2-hours and resulted in trace amounts of NH₃ generation (FIG. 4A), which could come from the urea residue. Similarly, wild-type C. necator (PHB producing, pBBRl-yfp) strain showed no C₂H₄ generation in the same test compared to the genetically engineered C2H4 producing strain (pBBRl-efe).

To prove the effects of zinc-histidine binding in in vivo ammonia production, nanorgs made from A. vinelandii DJ995 and A. vinelandii Wards42 (produces a nitrogenase without histidine tag) with CdS (without ZnS shell) and CZS (with two-monolayer ZnS shell) were also tested. As shown in FIG. 4b, without site-specific zinc-histidine coupling, ammonia yield is ⅓ to approximately ½ compared to the case with such binding.

12. ¹³C Isotope Labeling Tests.

¹³C isotope labeling tests were used to further prove biofuel production from inorganic ¹³CO₂. The tests were performed similar to the other nanorg tests, with the replacement of non-labeled CO₂ with ¹³C-bicarbonate. Typically, 20 mM NaH¹³CO₃ were supplied, with headspace charged with argon. Headspace gas was monitored using a mass spectrometer (Balzers Prisma QME-200) with QUADSTAR software. The fragments (m/q=30 and 47 for ¹³C2H₄⁺ and ¹³C₂H₅O⁺, respectively, FIG. 21G-I) indicate the production of ethylene and isopropanol with their corresponding nanorgs from ¹³CO₂.

B. Experimental Parameter Optimization for Improving Product Yield.

The efficiency for in vivo photocatalytic air-water or CO₂-water reduction using the QDs-living bacteria biohybrid (nanorgs) depends on many factors. These factors can be briefly classified as the intrinsic QDs part (light absorption, electronic transport, etc.), intrinsic living bacteria part (enzymatic activity, etc.), and the interactions between them (electronic coupling, cellular toxicity, etc.). Experimentally, factors including QDs surface functioning, QDs concentration, bacteria cell optical density, use of electron donor, irradiation intensity, etc. can be varied to obtain optimal product generation. The optimization was performed on NH₃ producing strains with nanorgs prepared from CZS1 QDs and A. vinelandii DJ995 bacterial cells. The results can guide the optimization of other QDs and bacteria strains for other fuel production.

1. Bacteria Cell Optical Density.

Firstly, in one embodiment, mixtures with variations in cell optical density (OD₆₀₀=0.1 to approximately 1.0) were tested, with 200 nM MPA-capped QDs in ASC5 media. Photocatalytic reactions were performed in air, with 400 nm light irradiation at 1.6 mW/cm² for 1 hour. As shown in FIG. 22A, NH₃ production increases with optical cell density, but not linearly. Optical density of 1.0 was used for further tests.

2. QDs Capping Ligands and QDs Concentration.

With fixed cell optical density (OD₆₀₀=1.0), QDs with different capping ligands (MPA, CYS, CA) and concentrations were tested. As presented in FIG. 22B, MPA and CA-capped QDs show a small increase of NH₃ yield with concentration (<200 nM) but drop down with higher QDs concentration (>500 nM). For CYS-capped QDs, a significant NH₃ production was observed with no further decrease at higher concentration. And generally, CYS-capped QDs show much higher NH₃ production compared to MPA and CA-capped QDs. This concentration and capping ligand-dependent NH₃ yield can be explained by the QDs uptake and cell viability. The cell viability-dependent NH₃ production is also demonstrated by performing the same tests in a pure nitrogen atmosphere. As shown in FIG. 22C-D, no change of NH₃ yield was seen when performing the assay in air or pure dinitrogen using MPA and CYS-capped QDs, which indicates that the nitrogen source (dinitrogen in the air or pure dinitrogen) is not a limiting factor. For CA-capped QDs, a significant decrease of NH₃ yield was observed when the same test was performed in air and compared to pure dinitrogen. This could be explained by the cell viability loss with CA-capped QDs, where the oxygen-sensitive nitrogenase is no longer protected by the bacteria cell, leading to a lower NH₃ yield.

3. Irradiation Intensity.

NH₃ production with varying light intensity (from 0.16 to 2.42 mW/cm² at reaction site) was also tested (500 nM CYS-capped QDs and OD₆₀₀=1.0 bacteria cells, 1 hour irradiation time).

Low NH₃ yield with low irradiation intensity is (FIG. 22E) due to the limitation of photo-induced electrons produced from QDs. On the other hand, a small decrease of NH₃ production with high-intensity irradiation could probably be related to the decrease of cell viability under strong near-UV light irradiation. In other tests, optimal light intensity (1.6 mW/cm²) was used. To further investigate the evolution of NH₃ with time under different light intensity irradiation (0.67 and 1.6 mW/cm²), the NH₃ production was monitored using both high and low photon flux. Though the generation of NH₃ is slower with lower light irradiation (FIG. 22), (FIG. 22F), the total NH₃ reached similar level after 4 hours. On the other hand, NH₃ production with high-intensity irradiation leveled off within 2 hours, and the accumulation of NH₃ (reaching the tolerance of this bacteria strain) can explain this saturation.

4. Effect of the Electron Donor.

To investigate the function of the sacrificial quencher (L-ascorbic acid, and sulfide here), we performed the same photocatalytic test in the different medias or buffers, (e.g. HEPES buffer ((e.g. 4-(2-hydroxyethyl)-1-piperazineethanesulfonic acid buffer)), MOPS (e.g. 3-(N-morpholino)propanesulfonic acid) buffer, PIPES (e.g. 1,4-Piperazinediethanesulfonic acid) buffer, PBS) with the removal of L-ascorbic acid, using the optimized CYS-capped QDs at 500 nM for 2 hours. As shown in FIG. 22G, lack of any sacrificial quencher, meant the NH₃ TON is about half compared to the same test in ASC5 or HEPES-S media. Furthermore, no significant difference in NH₃ yield was detected in different quencher-free medias. This could be explained by the sacrificial agent and its ability to facilitate electron injection to MFN and thus maintain cell viability.

To further understand the function of the sacrificial donor in photocatalytic NH₃ production, the time evolution of NH₃ was monitored in the media (HEPES) with or without the ascorbic acid. Interestingly, the total amount of NH₃ in the two tests reached the same level (FIG. 4e, FIG. 22H) regardless the existence of quencher after 3-hour reaction, which ruled out the necessity of using sacrificial quencher in photocatalytic NH₃ production. On the other hand, faster NH₃ generation (till the saturation point) was observed with the addition of ascorbic acid, which can be attributed to faster/easier hole removal (and hence facilitate electron transfer from the QDs to the nitrogenase). Overall, the sacrificial donor could have a more pronounced effect on reaction kinetics rather than thermodynamics.

Similar tests were also performed in Burk media and photocatalytic media supplied with 5, 10, and 25 mM L-ascorbic acid (ASC5, ASC10, and ASC25). As shown in FIG. 22I, FIG. S11I, NH₃ generation increases with time and levels off at 1.5 hours in ASC5 and ASC10 media. NH₃ yield is lower with ASC25 media but shows no saturation up to 4 hours, which can be explained by the lower cell viability due to L-ascorbic acid inhibition (lower yield) and the unlimited sacrificial hole quencher supply (no saturation). Furthermore, total ammonia production in Burk media is halved compared to ASC5 or ASC10 in the photocatalytic media. The growing cells (in Burk media) consumed the ammonia produced in the reaction resulting in lower photocatalytic ammonia production compared to the non-growing cells in the photocatalytic media. This phenomenon was also reported by Harwood et al., where methane production by photosynthetic bacteria R. palustris (light-driven CO₂ reduction by nitrogenase) is much higher with non-growing cells compared to growing cells³³.

C. Recovery Tests.

The saturation of NH₃ production could be due to the depletion of the sacrificial hole quencher (L-ascorbic acid) or the accumulation of NH₃ (an inhibitor for MoFe nitrogenase) in the reaction phase. To prove this hypothesis, the bacteria cells were separated by centrifugation and recharged with new QDs-containing media for photocatalytic reaction every 1.5 hours. Taking the cell loss due to centrifugation, the cell OD were re-measured before each cycle, and the NH₃ TON and recovery were normalized to the corrected cell OD (measured OD for the three cycles were 1.00, 0.728, and 0.565, respectively). NH₃ production was resumed after each cycle, and no obvious yield loss was seen (almost 100% recovery as shown in FIG. 23). This indicates that the reduction in the NH₃ production is due to the accumulation of NH₃ (as nitrogenase inhibitor), this could be resolved by using a continuous flow reactor or though constructing high NH₃ tolerant strains.

D. Extension to Other QDs and Bacteria Strains for Other Solar Fuel Production.

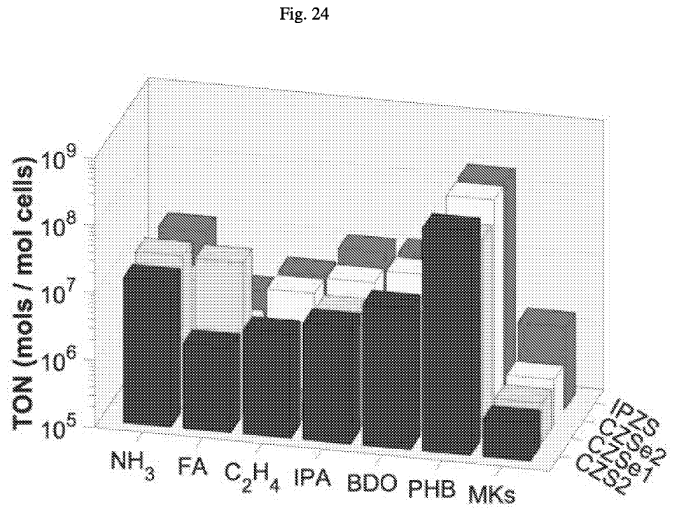

The knowledge obtained from the optimization of NH₃ production can be utilized to conduct other nanorgs tests with different combinations of QDs and bacteria strains, with the benefits of extending the light absorption spectra from near-UV, to visible, to near-IR using different QDs or different excitation sources, and obtain different fuels using different genetically modified strains. Some of the results are presented in FIG. 5 and FIG. 24.

E. Estimation of the Internal Quantum Efficiency (IQE).

The internal quantum efficiency (IQE), defined as the ratio of electron production to the total amount of photon absorbed, was estimated based on some of the following parameters:
a. NH₃ and H₂ turnover frequency: 8730 and 4350 s⁻¹, respectively.
b. CZS QDs: 500 nM concentration (c), the extinction coefficient at 418 nm ε(418)=385676 M⁻¹cm⁻¹ and from the UV-VIS of CZS QDs, ε(400)/ε(418)=0.85. Therefore, ε(400)=327825 M⁻¹cm⁻¹.
c. Cell OD₆₀₀=1.0, corresponding to 4.5×10⁸ cell/ml for Azotobacter vinelandii.
d. Light source: 400 nm LED, with 1.6 mW/cm² irradiation intensity (I).
e. Reactor: a glass vial with ˜ 1 cm inner diameter (d), with total reaction volume (V) of 1 ml. Therefore, the irradiation cross section S=πd²/4=0.52 cm² and the light path b=V/S=1.91 cm.
f. The cellular uptake of the CZS QDs is 14%.

Based on the Lambert-Beer's law, the light absorbed A=ε(400)bc=0.313, and the transmittance and can be calculated by A=2−log(% T), and T=48.6% and the absorbed part is 1−T=51.4%.

The incident photon number can be calculated from the irradiation intensity:

$$N = \frac{total energy}{photon energy} = \frac{intensity * area * time}{hc / λ} = \frac{ISt}{hc / λ}$$

And the incident photon flux (F_{inc}=N/t) is F_{inc}=^{IS}λ/hc=1.676×10¹⁴ s⁻¹.

The absorbed photon by the nanorgs is F_{abs}=F_{inc}×(1−T)×uptake=1.2×10¹⁴ s⁻¹.

The total electrons produced from the nanorgs can be calculated from the TOF of NH₃ (3 electrons per NH₃ molecule) and H₂ (2 electrons per H₂ molecule), and for one cell, the electron flux is F_{e/cell}=3×TOF(NH₃)+2×TOF(H₂)=34890 s⁻¹.

With OD₆₀₀=1.0 cell in 1 ml total volume, the total electron flux is Fₑ=1.5×10¹³ s⁻¹.

Therefore, IQE=Fₑ/F_{abs}×100%=13.1%.

F. Scaled-Up PHB Production in a Photobioreactor.

To investigate the scaling up of solar-biofuel production, the nanorg test scale was amplified from 5 ml to several liters. The scaled-up production was conducted in a benchtop bioreactor (BioFlo/CelliGen 115). The fermentation (approximately 4 L) was conducted in the MSM media with 200 nM Cys-capped QDs (CZS2 in this case). Wild-type C. necator pBBRl-yfp strain obtained from FGN media (OD₆₀₀ between 2 and 3) was washed twice with MSM media and re-suspended in the fermentation media with an initial OD₆₀₀ of 1.695. The media was flushed with CO₂/O₂ mixture (4:1, 0.5 SLPM (standard litre per minute)) for 15 min before irradiation. The temperature was maintained at 30° C., and the agitation was kept at 200 rpm. The pH was monitored during the whole process (approximately 6.0), and CO₂/O₂ was recharged every 12 hours. Five 400 nm LED panels (2 on the side wall and 3 at the bottom) were used as irradiation source. The fermentation was stopped after 68 hours, and the nanorgs were collected using centrifugation (7,000 rpm). After washing twice with D.I. water, PHB from the nanorgs was extracted with sodium hypochlorite/chloroform mixture (35) and the chloroform layer obtained from centrifugation was pulled to 70% methanol for precipitating the PHB, which were collected and dried in 70° C. overnight. The whole process was shown in FIG. 24. The dry PHB, fermentation samples before and after irradiation were subjected for GC (gas chromatograph) analysis to evaluate the recovery and purity of the PHB. Here, we obtained a total of 1.0 gram of PHB, with purity of approximately 88% (GC analysis). GC analysis of the cells yields a total PHB of 637.5 mg and 1105 mg before and after irradiation (recovery approximately 80%), indicating a net 467.5 mg production of PHB (converting CO₂ to PHB with light-activated nanorgs). Using the PHB solution (dissolved in glacial acetic acid with gentle heating), we have demonstrated the use of PHB for making a plastic thin film with simple casting.

For scaling up the process for commercial production, our process in lab scale (several liters) is amenable to a pilot scale plant (approximately 1000 L), and eventually even to a commercial level (>40,000 L) with little change of the configuration. The cells with biofuel or bioproduct can simply be filtered using commercial membranes, reuse the buffered water, and simply lyse the cells using detergent (sodium dodecyl sulfate) solution, followed by a charged filtration membrane to capture/recycle all the QDs (due to their surface charge). Once QDs are filtered, the remaining solution with biodegradable plastic, biofuel, or other specialty chemicals as bioproducts can be precipitated (as depicted in FIG. 6) to obtain the final product, and then packaged/formed into a product, to directly convert CO₂ into fuels/biodegradable plastics.

TABLE 3
Abbreviations.

| | |
|---|---|
| (TMS)₃P | 1. Tri s (trimethylsilyl)phosphine |
| 2,4-DNP | 2. 2,4-dinitrophenol |
| AGE | 3. Agarose gel electrophoresis |
| ATP | 4. Adenosine 5′-triphosphate |
| ATR | 5. Attenuated total reflectance |
| BM | 6. Burk media |
| CA | 7. Cysteamine |
| CB | 8. Conduction band |
| CdX | 9. CdS or CdSe QDs, X = S or Se |
| CFU | 10. Colony forming unit |
| CL | 11. Cell lysate |
| CPK | 12. Creatine phosphokinase |
| CYS | 13. L-cysteine |
| CZS | 14. CdS@ZnS core-shell QDs |
| CZSe | 15. CdSe@ZnS core-shell QDs |
| CZTS | 16. Cu₂ZnSnS₄@ZnS QDs |
| DDT | 17. 1-dodecanethiol |
| DPV | 18. Differential pulse voltammetry |
| DTT | 19. Sodium dithionite |
| EDTA | 20. Ethylenediaminetetraacetic acid |
| EIS | 21. Electrochemical impedance spectroscopy |
| FTIR | 22. Fourier-transformed infrared spectroscopy |
| FTO | 23. Fluorinated-tin oxide |
| GC | 24. Gas chromatography |
| ICP-MS | 25. Inductively-coupled plasma-mass spectrometry |
| IMAC | 26. Immobilized metal affinity chromatography |
| IPZS | 27. InP@ZnS core-shell QDs |
| LED | 28. Light emission diode |
| LN2 | 29. Liquid nitrogen |
| LPM | 30. Liter per minute |
| MFN | 31. MoFe nitrogenase |
| ML | 32. Monolayer |
| MOPS | 33. 3-(N-Morpholino)propanesulfonic acid |
| MPA | 34. 3-mercaptopropionic acid |
| NADPH | 35. Nicotinamide adenine dinucleotide phosphate, reduced |
| NHE | 36. Normal hydrogen electrode |
| OA | 37. Oleic acid |
| OCP | 38. Open circuit potential |
| OD | 39. Optical density |
| ODE | 40. 1-octadecene |
| OLA | 41. Oleylamine |
| PBS | 42. Phosphate-buffered saline |
| PIPES | 43. 1,4-Piperazinediethanesulfonic acid |
| PL | 44. Photoluminescence |
| PMSF | 45. Phenylmethylsulfonyl fluoride |
| QD | 46. Quantum dot |
| QDs-CL | 47. Quantum dots-cell lysate mixture |
| QDs-MFN | 48. Quantum dots-MoFe nitrogenase biohybrid |
| rpm | 49. Revolutions per minute |
| SDS—PAGE | 50. Sodium dodecyl sulfate-polyacrylamide gel |
| | electrophoresis |
| TBP | 51. Tributylphosphine |
| TCD | 52. Thermal conductivity detector |
| TES | 53. N-[Tris(hydroxymethyl)methyl]-2-aminoethanesulfonic |
| | acid |
| TOF | 54. Turnover frequency |
| TON | 55. Turnover number |
| Tris | 56. Tri s (hydroxymethyl)aminomethane |
| UHP | 57. Ultrahigh purity |
| UPC | 58. Ultrapure carrier |
| UV-VIS | 59. Ultraviolet-visible |
| VB | 60. Valence band |

Described herein are compositions and methods used during the development of the present inventions, including but not limited to embodiments of: Quantum dots (QDs) Synthesis; CdS and CdSe QDs; InP@ZnS QDs (IPZS); Cu2ZnSnS4 QDs (CZTS); ZnS shell growth; Ligand exchange; Characterization of QDs, including but not limited to: Optical spectroscopy; Elemental analysis, Electrochemical analysis, and Zeta potential measurements; Cellular enzyme preparation and characterization; Azobacter vinelandii DJ995 bacteria growth and cell lysate preparation; Cell lysate (CL) activity determination; MoFe nitrogenase (MFN) purification; Cupriavidus necator (C. necator) culture; Analysis of product generated from C. necator culture; QDs-enzyme complex preparation and characterization; UV-VIS determination of QDs-protein binding; Fourier-Transformed Infrared Spectroscopy (FTIR); SDS-PAGE protein electrophoresis; Agarose Gel Electrophoresis (AGE); Inductively-coupled plasma mass spectroscopy (ICP-MS); QDs-MFN biohybrid for light-induced proton reduction; Light-induced redox reaction with QDs-CL mixture; Interactions between QDs and the living bacteria; Cellular QDs uptake assay; Laser-scanning confocal microscopy; Cell growth curve measurement; Resazurin dye cell viability assay; Colony forming unit (CFU) assay; Formation of QDs-living bacteria nano-biohybrid (nanorgs) for light-induced air-CO2-water to fuel production; Experiment and Control Groups; Experimental parameter optimization for improving product yield; Recovery tests; Extension to other QDs and bacteria strains for other solar fuel production; Estimation of the internal quantum efficiency (IQE); and Scaled-up PHB production in a photobioreactor.

Results described herein include but are not limited to embodiments of: UV-VIS spectra and photoluminescence (PL) spectra of the QDs; Electrochemical characterization of the QDs; Zeta potential CYS-capped CZS; Natural growth and production with different C. necator strains; Proof of QDs and cellular protein binding; Light-induced H2 or NH3 production with QDs-MFN or QDs-CL biohybrids; Laser scanning confocal images; Cell growth curve assay; Cell viability tests with resazurin; Control experiments with A. vinelandii and C. necator; Optimization of experimental parameters for improved NH3 production; Recovery test for NH₃ production; TON of different fuels; and Scaled-up production of PHB, etc.

TABLE S1
Total Cd and Zn (in ppb) determined from ICP-MS and
determination of the thickness of the ZnS shell (Real layer number).

| CdS@ZnS | Total Cd (ppb) | Total Zn (ppb) | Dₜₒₜₐₗ (nm) | Real layer (ML) |
|---|---|---|---|---|
| 0 ML | 469720 | 1427 | — | — |
| 1 ML | 94441 | 25677 | 3.94 | 0.6 |
| 2 ML | 38230 | 46066 | 4.91 | 2.2 |
| 3 ML | 243618 | 577668 | 5.74 | 3.5 |

TABLE S1-2
First exciton peak position, extinction coefficient and real
thickness of CdS@ZnS nanoparticles.

| | First Exciton Peak | Extinction Coefficient |
|---|---|---|
| CdS@ZnS | (nm) | (microM⁻¹cm⁻¹) |
| OML | 405 | 0.491 |
| 1 ML | 418 | 0.400 |
| 2 ML | 418 | 0.386 |
| 3 ML | 418 | 0.386 |

TABLE S2
Core-shell (ZnS, 2ML) extinction coefficient at individual specific wavelengths.

| | CZS1 | CZS2 | CZSel | CZSe2 | CZSe3 | IPZS | CZTS |
|---|---|---|---|---|---|---|---|
| wavelength | 418 | 437 | 500 | 525 | 580 | 610 | 400 |
| (nm) | | | | | | | |
| Extinction | 385,676 | 573,697 | 68,488 | 67,792 | 120,000 | 1,272,300 | ~10⁴ |
| coefficient | | | | | | | |
| (M⁻¹ cm⁻¹) | | | | | | | |

TABLE S3
Bandedge and bandgap information of CdS and CdSe QDs obtained
from optical (UV-VIS) and electrochemical (DPV) measurement.

| | E_{CB} (V) | E_{VB} (V) | E_{g, ec} (eV) | E_{g, op} (eV) | D (nm) |
|---|---|---|---|---|---|
| CdS1 | −0.83 | 2.25 | 3.08 | 3.06 | 3.55 |
| CdS2 | −0.75 | 2.20 | 2.95 | 2.94 | 4.17 |
| CdS3 | −0.61 | 2.21 | 2.82 | 2.79 | 5.06 |
| CdSe1 | −0.72 | 1.89 | 2.61 | 2.50 | 2.30 |
| CdSe2 | −0.53 | 1.85 | 2.38 | 2.38 | 2.59 |
| CdSe3 | −0.31 | 1.88 | 2.19 | 2.07 | 4.58 |

Note:
E_{CB}, E_{VB} (vs. NHE) are the conduction band and valence band position, respectively, from DPV measurements. E_{g, ec} is the electrochemical bandgap determined from the conduction and valence band position (E_{g, ec} = E_{VB} − E_{CB}). E_{g, op} is the optical bandgap from UV-VIS measurements (E_{g, op} (eV) = 1239.8/λ (nm), where λ is the wavelength of the first exciton peak). D is the diameter of the nanoparticles determined from the optical bandgap.

TABLE S4
Carrier lifetime and coefficient extracted from a
bi-exponential fit of the OCP decay.

| CdS@ZnS | A₁ (V) | t₁ (s) | A₂ (V) | t₂ (s) |
|---|---|---|---|---|
| 0 ML | −2.301 | 1482 | −0.158 | 5680 |
| 1 ML | −46.82 | 271.7 | −0.0604 | 4734 |
| 2 ML | −1.190 | 544.0 | −0.204 | 3958 |
| 3 ML | — | — | — | — |

TABLE S5
Cell lysate activity for enzymatic proton and dinitrogen-water reduction.

| | Product | Cell Lysate Activity (nmol/ml CL/min) |
|---|---|---|
| Proton reduction | H₂ | 260.4 |
| Dinitrogen-water | H₂ | 108.7 |
| reduction | NH₃ | 112.5 |

TABLE S6
Primers used for plasmid construction

| Primer | Sequence (5'---->3′) |
|---|---|
| U-ara-F | 1. gggaaagUaacgtta |
| | tgacaacttgacggc |
| | tac |
| U-ara-R | 2. atatctccUtcttaa |
| | aagatcttttgaatt |
| | ccc |
| U-alsS-F | 3. aggagataUacatat |
| | gaccaaggccaccaa |
| | ggaacag |
| U-alsS-R | 4. acttaacUagcgtac |
| | gtcacagcgccttgg |
| | tcttcatcagc |
| U-alsD-F | 5. agttaagUataagaa |
| | ggagatataacatga |
| | agcgcgagtcgaaca |
| | tccag |
| U-alsD-R | 6. atggttgUcctcctt |
| | tctcgagtcattccg |
| | gcgagccctcg |
| U-sADH-F | 7. acaaccaUgaagggc |
| | ttcgccatgctg |
| U-sADH-R | 8. ggagacaUcctaggt |
| | cacaggatcaccacg |
| | gccttg |
| phaC-up-F | 9. acgcgccgatgaaca |
| | ggtc |
| phaB-dn-R | 10. tgctcatcatgccct |
| | gcatcatcg |
| phaC-up-sacI-F | 11. ttattgagctcacgc |
| | cggtcgcttctactc |
| | ctatc |
| phaB-dn-pacI-R | 12. attatattaattaat |
| | cgatgtagttgctca |
| | tcatgccctg |
| phaCB-ov-speI-F | 13. acggcagagagacaa |
| | tcaaatcactagtcc |
| | taggcctgccggcct |
| | ggttcaaccag |
| phaCB-ov-speI-R | 14. ctggttgaaccaggc |
| | cggcaggcctaggac |
| | tagtgatttgattgt |
| | ctctctgccgt |
| U-bktB-F | 15. aggagataUacatat |
| | gacccgtgaagttgt |
| | tgttg |
| U-bktB-R | 16. aacttctcctUtacg |
| | tacgttagatacgtt |
| | caaaaattgctgcaa |
| | tacc |
| U-ctfAB-F | 17. aaggagaagtUacca |
| | tgaacaaggtgatca |
| | cggacc |
| U-ctfAB-R | 18. acttaactagcUcga |
| | gtcacagatgcacct |
| | cgaactcg |
| U-adc-F | 19. agctagttaagUata |
| | agaaggagatataac |
| | atgctgaaggacgag |
| | gtgatc |
| U-adc-R | 20. atggttgUcctcctt |
| | tggatcctcacttca |
| | gatagtcgtagatca |
| | cttcgg |

TABLE S7
Error bars for ratio of light-driven production
to natural production (in percentage).

| | PHB | C2H4 | MKs | IPA | BDO |
|---|---|---|---|---|---|
| CZS2 | 167 ± 18 | 106 ± 5 | 26 ± 2 | 12 ± 1 | 20 ± 1 |
| CZSe1 | 95 ± 7 | 3.2 ± 0.2 | 32 ± 1 | 12 ± 1 | 10 ± 1 |
| CZSe2 | 148 ± 18 | 78 ± 5 | 35 ± 3 | 17 ± 1 | 19 ± 2 |
| IPZS | 148 ± 16 | 86 ± 8 | 156 ± 14 | 25 ± 2 | 19 ± 2 |

VIII. Quantum Dot-Azotobacter vinelandii Living Nano-Biohybrid Organisms Cause Light-Driven Air-Water Reduction: Solar-Powered Living Factories.

Many naturally occurring and synthetic bacteria can accomplish industrially relevant reactions, like, for e.g., conversion of nitrogen to ammonia in ambient conditions, using chemical energy to generate electrons and reduce readily available chemical feedstocks, and can be labeled as living factories. Refs. 1-6 However, they derive the chemical energy needed sometimes from valuable food stocks, thereby reducing their attraction for energy conversion to useful solar or biofuels. Inorganic photocatalysts directly derive energy from sunlight to generate photoelectrons for reduction of inexpensive and abundant chemical feedstocks like, for e.g., air, water, and carbon-dioxide, but their lack of selectivity, low efficiency, and sometimes use of conditions such as high-temperature and pressure limit their widespread application. Refs. 7-18.

Combining these desired functionalities of direct stimuli-activations via light, voltage, or magnetic field, with the versatility of designing desired synthetic metabolic networks in living cells can provide an unprecedented platform for designing and creating multifunctional living nano-biohybrid organisms (or nanorg's), and for specific applications as living solar-powered factories for direct energy conversion to solar fuels. Ref^{19.}

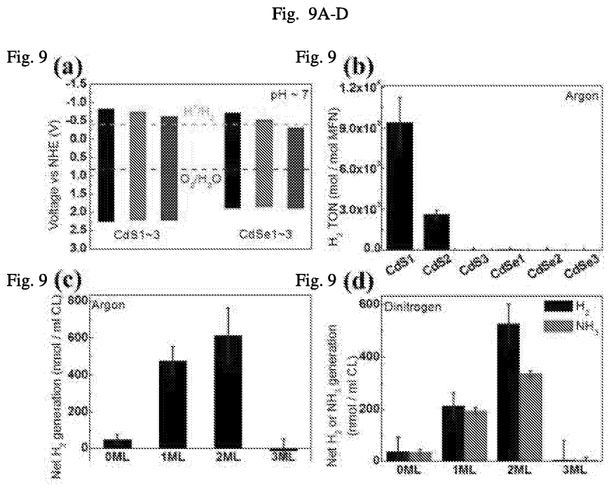

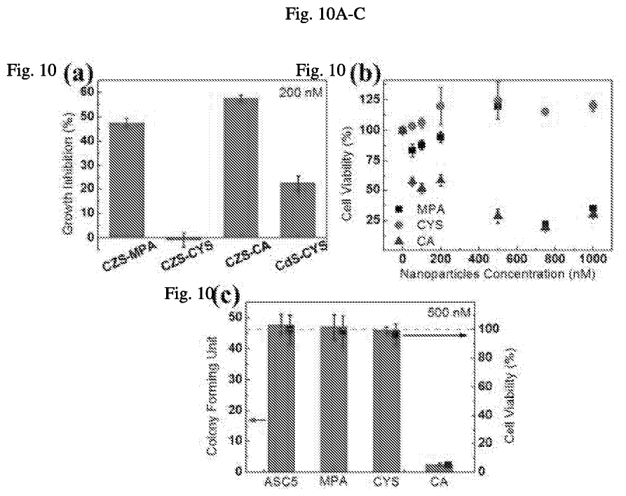

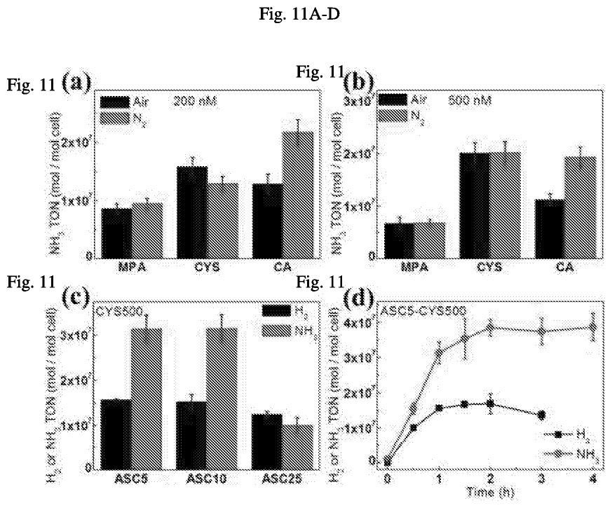

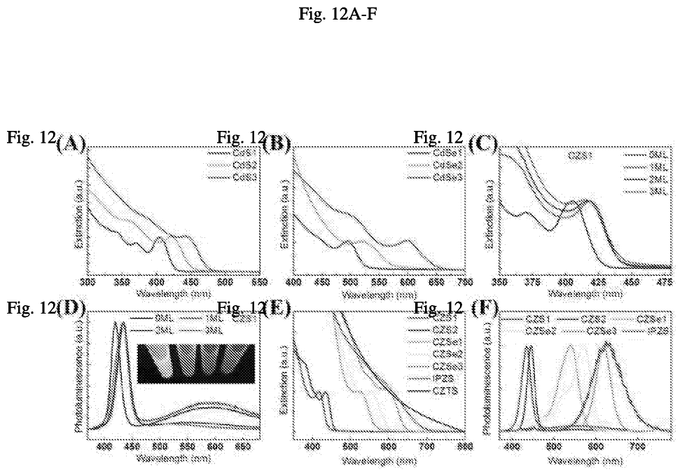

One initial step towards development of such living organisms is chemical coupling, site-specific self-assembly²⁰⁻²⁴ from dispersion, and energetic coupling between QDs and synthetic bacteria by appropriately choosing QD size and material (core-shells, if different materials required for energetic alignment and chemical coupling/biocompatibility), QD surface charge and ligands, and desired site-specific attachment. To ensure good energetic alignment and efficient electron injection from the conduction band of photoexcited QDs to molybdenum-iron nitrogenase (MFN) enzyme in Azotobacter vinelandii²⁵ for multielectron reduction of water to hydrogen, we conducted in-situ experiments with MFN from cell-lysate with different cadmium chalcogenide QDs. Since the choice of chalcogen virtually fixes the valence band state and any change in size and therefore quantum confinement tunes the conduction band position,²⁶ we identified different sizes of cadmium sulfide (CdS, FIG. 12a) and cadmium selenide (CdSe, FIG. 12b) QDs can energetically match the reduction potential for the MFN enzyme. Using detailed electrochemical measurements²⁷⁻²⁹ of these QDs, we identified that 3.6, 4.2, and 5.0 nm diameter CdS QDs had the desired electrochemical alignment (FIG. 9a), and while small CdSe QDs (2.3 nm) also had desired electrochemical potential, poor charge injection efficiency of photogenerated electron, strong electron-hole recombination, and smaller electron lifetime lead to lower number of photogenerated electrons from CdSe NPs to be utilized by MFN enzyme (FIG. 13A-D, 13J, 13E, 13K, 13F). Consequently, using water reduction and generation of hydrogen in these in-situ measurements, we observed much lower photocatalytic activity from these nano-biohybrids (FIG. 9b, FIG. 26A-B).

To identify suitable chemical coupling (and ensure biocompatibility, discussed later) and design site-specific attachment and self-assembly in chosen QDs, we tested large CdS and ZnS nanoparticles. These particles were suspended with cell lysate prepared from Azotobacter vinelandii DJ995 bacteria followed by separation and the resulting protein-bound particles were analyzed using gel electrophoresis (SDS-PAGE) to identify the type of enzymes attached to the nanoparticle surface (FIG. 2AB). While CdS nanoparticles showed non-specific attachment of different enzymes, ZnS nanoparticles selectively attached MFN, thereby identifying the suitable choice of material for the QD surface-MFN interaction. We synthesized CdS QDs with different ZnS shell thickness (FIG. 12C-D) and conducted in-situ testing of biohybrid formed based on the coordination between zinc ion and histidine (Histidine-tagged MFN covalently binds to the ZnS surface of the CdS@ZnS core-shell nanoparticles) under 400 nm light irradiation. Using water reduction to hydrogen and dinitrogen-water reduction for hydrogen/ammonia production, different ZnS shell thickness demonstrated thickness-dependent photocatalytic performance. ZnS shell thickness affects the photogenerated electron transport characteristics and kinetics in CdS core. While increased shell thickness causes surface passivation to reduce surface states/defects, it also serves as a barrier for charge injection of photogenerated electron from the core. For the optimal design of biohybrid, CdS@ZnS core-shell QDs with nominal x monolayers (x=0˜3) of ZnS shell and 3-mercaptopropionic acid (MPA) capping ligand were used to realize formation of MFN biohybrid in pH 7.4 media. Using our in-situ testing of hydrogen and ammonia generation under irradiation, with L-ascorbic acid or HEPES as sacrificial agent, significant enhancement of water and dinitrogen reduction (FIG. 27A, FIG. 28A-B) was observed in 1 and 2 monolayer thick ZnS shell, with maximum 615 nmol/ml cell lysate/h hydrogen generation rate in water reduction and 527/337 nmol/ml cell lysate/h hydrogen/ammonia generation rate in dinitrogen-water reduction for 2 monolayer thick CdS@ZnS QD-MFN biohybrid. As a comparison, QDs without ZnS shell MFN enzyme attachment show negligible yield. The selective binding of Ffis-tagged MFN to zinc-rich nanoparticle surface is also confirmed by control experiments (FIG. 27B, FIG. 27c), where addition of imidazole (coordinates with Zn²) or increasing media acidity (protonates histidine) inhibits such interactions and hence no hydrogen production by the biohybrid was observed (same as using QD as control). Optimal design of 2 monolayers thick CdS@ZnS QD-MFN biohybrid was also evident by electrochemical impedance spectroscopy (FIG. 13I, L, G, M), small total capacitance and charge transport resistance), open-circuit potential decay (FIG. 13I, Table S4, reduced non-radiative charge recombination) and photoluminescence (FIG. 12C-D, removal of surface states), showing the importance of simultaneous optimization of surface tuning, photophysics, and charge tunneling (across QD-shell) in designing highly efficient QD-MFN biohybrids.

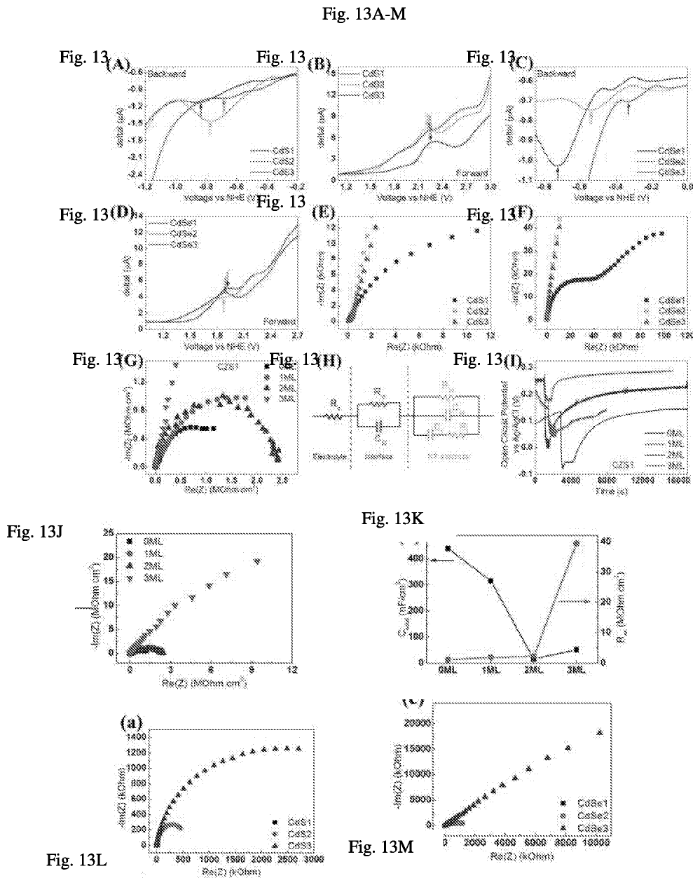

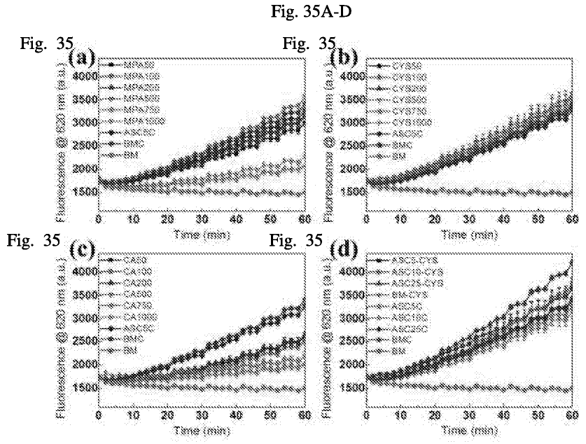

Another requirement for making living nano-biohybrid nanorg's was cell uptake³⁰⁻³³ and viability³⁴⁻³⁹ of designed QDs. An aspect of this, besides biocompatibility of ZnS coating, is the ligand and charge on QD surface. Using three-different similar-sized QD ligands with different surface charge: mercaptopropionic acid (MPA, negative charge), cysteamine (CA, positive charge) and cysteine (CYS, zwitterion), we tested cell viability of CdS@ZnS QDs using three-different methods.⁴⁰⁻⁴² First, using cell growth (monitored using optical density) in the growing media (Burk media) with nanoparticles, we have observed high growth inhibition for MPA- and CA-coated nanoparticles (FIG. 10a, FIG. 29), but no such inhibition for CYS-capped QDs even at high concentrations (similar to no treatment). Under light irradiation with non-growing nanorg cells in the photocatalytic media (FIG. 31), cell viability is almost not affected with CYS-coated nanoparticles and low concentration MPA-coated nanoparticles. With higher concentration MPA-coated nanoparticles and even low (50 nM) concentration of CA-coated QDs, a significant decrease in cell viability was observed. Low cell viability renders the cell unable to remove the oxygen in the air which causes deactivation of MFN enzyme reaction center leading to low ammonia yield (confirmed by conducting the same test in pure dinitrogen atmosphere and ammonia production was increased by almost one fold with CA-coated nanoparticles, FIG. 11a, FIG. 11b). The second measure of cell viability used was resazurin dye assay, which also demonstrated high cell viability for zwitterion and negative-charged QDs (FIG. 10b, FIG. 35). A more detailed investigation with colony forming unit analysis (CFU) also showed same results (FIG. 11c), with the highest viability for zwitterion and negatively charged QDs, followed by a strong reduction in the number of viable cells with CA-coating. While the cellular uptake with positively charged CA-coated QDs was much higher than negative or zwitterions ligands with similar sizes, strong non-specific attachment of QDs to negatively-charged cell organelles (like, for e.g., DNA, RNA, proteins) could be responsible for low cell viability, especially at high CA-coated QD concentrations.

Following design and self-assembly of appropriate living QD-Azotobacter vinelandii biohybrid nanorg's (CdS@ZnS with 2 monolayer shells, with cysteine ligand coating and site-specific attachment with histidine-tagged MFN enzyme), we tested their ability to fix light-energy into specific bonds using inexpensive chemical feedstocks like, for e.g., air and water. Optimized bacteria cell optical density (FIG. 36, OD₆oo=L0), QD concentration (FIG. 37), and irradiation intensity (FIG. 39, 1.6 mW/cm²) were used for tests, described herein.

While different capping ligands with CdS@ZnS QDs lead to different optimal QD concentrations for improved ammonia production (FIG. 38), due to different uptake and cell viability, the site-specific attachment of optimally designed CYS-coated CdS@ZnS (2 monolayer) QDs leads to moderate uptake compared to similar CA-capped QDs, but higher yield of ammonia generation with CYS-coated QDs (compared to MPA or CA ligands, FIG. 38), due to dual effects of cell viability and uptake with different ligand capping and resulting QD surface charge. With cysteine-coated CdS@ZnS2ML nanoparticles at 500 nM with cell optical density (OD600) at 1.0 and under 1.6 mW/cm² 400 nm light irradiation, hydrogen and ammonia production (using solar-driven air-water reduction) were also monitored in photocatalytic media with different L-ascorbic concentration (5, 10, 25 mM). The hydrogen generation (FIG. 42A-B) saturated at 1.5 hours, similar to the case of ammonia production. Comparison between photocatalytic reaction with QD and nanorg's show an interesting L-ascorbic acid concentration-dependent yield. Hydrogen production is higher with the nano-biohybrid mixture at the low L-ascorbic acid level, but surpassed by pure nanoparticles at higher L-ascorbic acid concentration, due to faster quench of photogenerated holes and decrease in cell viability with higher L-ascorbic acid concentrations. Ammonia yield (FIG. 40A) is also higher in media with lower L-ascorbic acid concentration. Turnover frequency calculated in the first 1 hour (linear accumulated of ammonia and hydrogen with time) is 8.73×10³ s″¹ and 4.35×10³ s″¹ for ammonia and hydrogen generation (in ASC5 media), respectively (FIG. 11d).

In conclusion, we have demonstrated the formation of a living QD-Azotobacter vinelandii DJ995 nano-biohybrid nanorgs via the design of appropriate QDs and facile mixing, self-assembly, and site-specific attachment of desired nanorg's. Based on the success of in-vitro testing, photocatalytic living cell ammonia and hydrogen production are realized in-vivo through air-water and water reduction using light irradiation in non-growing cells. We have shown the importance of optimal QD material and size design due to alignment and charge injection of a photogenerated electron to MFN-enzyme, the function of biocompatible ZnS shell in site-specific Histidine-tagged MFN enzyme binding, charge transport tuning, CdS cytotoxicity reduction. The cysteine-coated CdS@ZnS (2 monolayers thick) QDs showed sufficient cell uptake and cell viability of nano-biohybrids, to facilitate high-efficiency and high-selectivity in-vivo photocatalytic production of ammonia and hydrogen with an optimized turnover frequency (TOF) of 8.73×10³ s″¹ and 4.35×10³ s″¹, respectively. This could pave the way of designing highly efficient solar-powered living factories for solar fuel and solar fertilizer generator, using readily available chemical feedstocks. Furthermore, this idea could be extended as a platform technology to design, synthesize, and test other engineered living nano-biohybrid systems, with different combinations of semiconductor nanomaterials and synthetic microorganisms, to harness the power of desired biological processes with multifunctional properties of designer materials like, for e.g., external stimuli-activation with light, electrical pulses, or magnetic field, to wireless communication with living cells.

### IX. Gold Nanoclusters Cause Selective Light-Driven Biochemical Catalysis in Living Nano-Biohybrid Organisms.

Living nano-biohybrid organisms or nanorgs combine the specificity and well-designed surface chemistry of an enzyme catalyst site, with the strong light absorption and efficient charge injection (for biocatalytic reaction) from inorganic materials. Previous efforts in harvesting sunlight for renewable and sustainable photochemical conversion of inexpensive feedstocks to biochemicals using nanorgs focused on the design of semiconductor nanoparticles or quantum dots (QDs). However, metal nanoparticles and nanoclusters (NCs), such as gold (Au), offer strong light absorption properties and biocompatibility for potential application in living nanorgs. Here we show that optimized, sub-1 nanometer Au NCs-nanorgs can carry out selective biochemical catalysis with high turnover number (10⁸ mol/mol of cells) and turnover frequency (>2×10⁷ h⁻¹). While the differences of size, light absorption, and electrochemical properties between these NCs (with 18, 22, and 25 atoms) are small, large differences in their light-activated properties dictate that 22 atom Au NCs are best suited for forming living nanorgs to drive photocatalytic ammonia production from air. Further, by comparing the light-driven ammonia production yield between strains producing Mo—Fe nitrogenase with and without histidine tags, we demonstrate that preferential coupling of Au NCs to the nitrogenase through Au-histidine interactions is crucial for effective electron transfer and subsequent product generation. Together, these results provide the design rules for forming Au NCs-nanorgs, and may have implications for carrying out light-driven biochemical catalysis for renewable solar fuel generation.

Biochemical conversion of inexpensive feedstocks like, for e.g., air, water, and carbon dioxide (CO₂) into desired chemicals and fuels offers specificity and low cost, but typically requires energetic substrates such as sugar to supply the energy required for conversion. Inorganic catalysts can directly utilize sunlight for photocatalytic conversion at high efficiencies, but suffers from lack of specificity.¹⁻³ Recently, nano-biohybrid catalysts have been suggested an alternative, to combine the best properties of high turnover, efficiency, and selectivity, in a single biocatalyst both using in vitro⁴⁻⁷ and in vivo⁸⁻¹⁰ studies.

Moreover, living or whole-cell biohybrids offer additional advantage of self-replication or growth, avoiding enzyme deactivation, and enzyme regeneration and repair. Living nano-biohybrid organism, or nanorgs, combine these functionalities as a platform where targeted interfacial chemistry and specific enzyme attachment can be used to ensure facile uptake, self-assembly, and light-driven catalysis to specific chemicals or solar fuels.¹⁰

Further, these nanorgs do not require any energetic substrates like, for e.g., sugars, glucose, or NADH, and can directly trigger an energetically uphill biocatalytic transformation of inexpensive substrates (air, CO₂) using wireless transfer of energy from light (or sunlight), for a range of selective biochemical or fuel generation as living microbial factories. To further advance nanorgs beyond semiconductor nanoparticles, here we demonstrate nanorgs made from small, sub-1 nanometer Au NCs.¹¹⁻¹³ Besides the biocompatibility of Au and their large extinction coefficient,¹⁴⁻¹⁶ this work will also serve to advance expanding the nanorg platform to a wide range of materials and microbial platforms.

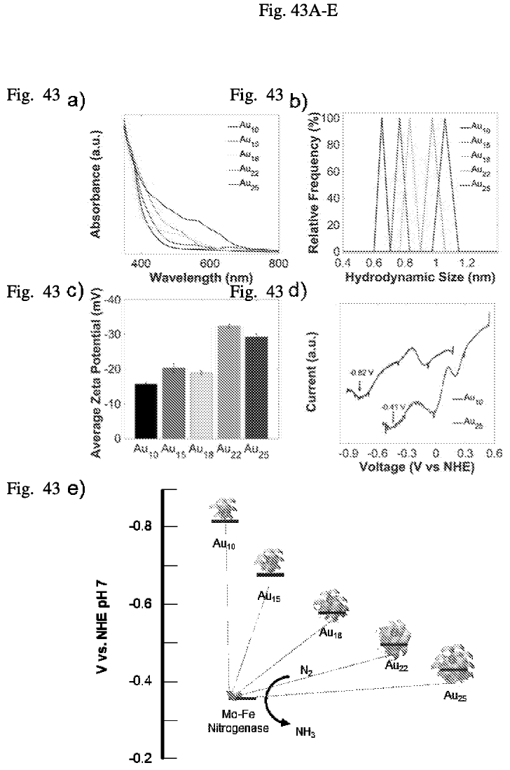

As described herein, nanorgs were engineered by screening different Au NCs¹⁷⁻²⁰ for biocompatibility with exemplary bacterial strains described herein. For providing good biocompatibility, facile uptake, low hydrodynamic radius, and efficient removal/clearance of unattached Au NC, we focused on Au NCs capped with the zwitterionic ligand, glutathione (GSH).²¹⁻²³ These atomically-precise Au NCs were synthesized using simple wet-chemistry, with tunable optoelectronic properties (FIG. 43a), hydrodynamic size (FIG. 43b), surface charge (FIG. 43c), and electrochemical properties (FIG. 43d) by modifying experimental factors, e.g. reaction pH, reducing agents, or reaction time. While these glutathione-capped Au NCs have similar sub-1 nm size, their optoelectronic and electrochemical properties, e.g. HOMO-LUMO state energies, bandgaps etc. were slightly different. To further understand how these differences in light absorption, charge injection could affect the production yield, we focused on visible or near-infrared light-activated Au NCs with 18, 22, and 25 Au atoms (labeled as Au₁₈, Au₂₂, and Au₂₅). The nitrogen-fixing bacteria, Azotobacter vinelandii, were selected to couple to these Au NCs to form Au NC-A. vinelandii nano-biohybrids. Under visible light irradiation, photoexcited electrons (from Au NCs) with suitable redox potential can directly inject to the nitrogenase in A. vinelandii, followed by conversion of dinitrogen to ammonia in ambient air (FIG. 43e).

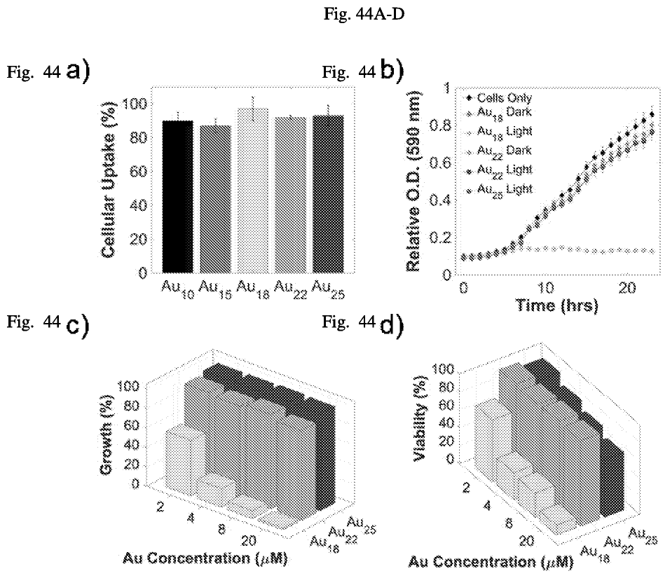

To allow direct electron transfer from light-activated Au NCs to the intracellular nitrogenase enzyme, cellular uptake and biocompatibility of these nanoclusters are required. Due to their extremely small sizes and favorable surface charge (zwitterionic), intracellular incorporation of these nanoclusters was as high as 90% (FIG. 44a). An additional merit of using the A. vinelandii DJ995 strain (produces a His-tagged nitrogenase) is the strong Au NCs-nitrogenase coupling facilitated by the Au-histidine interactions (to be discussed later). Due to their similar hydrodynamic size and surface charge, these nanoclusters demonstrated similar cellular uptake for A. vinelandii (FIG. 44a). However, significant differences in their biocompatibility were observed, under dark and light-activated conditions. By treating A. vinelandii with Au₂₂ NCs in dark or with light irradiation, we observed almost no effect on growth inhibition (as shown in the growth curves, FIG. 44b, 44c), or loss of cell viability (after 5 hour light irradiation, as shown in the resazurin assay in FIG. 44d) even at very high NC concentration (up to 20 μM). Similarly, Au₂₅ (FIG. 44b-d) and the smaller Au₁₀₋₁₂, Au₁₅ NCs (FIG. 47A-C) demonstrate high biocompatibility with minor loss in cell viability. While A. vinelandii show no biocompatibility issues with Au₁₈ NCs when treated in dark, light-treatment renders up to 80% growth inhibition and 95% loss of cell viability. This difference indicates that Au₁₈ NCs were not suitable for building living nanorgs.

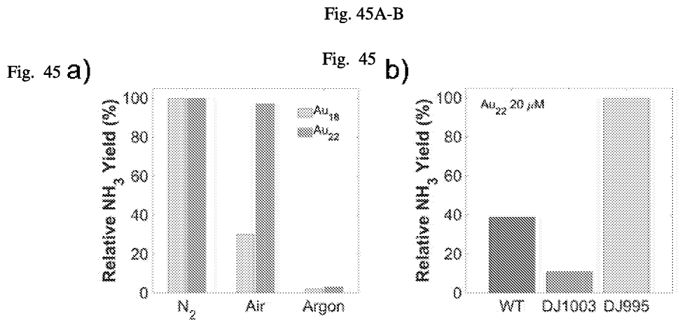

To further probe the effect of cell viability and coupling chemistry between Au NCs and nanorg enzymes, we carried out light-driven ammonia production using these Au NCs-A. vinelandii nanorgs in sugar-free media. This reaction relies on the injection of light-induced electrons from Au NCs to the nitrogenase in the bacteria cells. Since nitrogenase is extremely sensitive to oxygen and is normally protected inside living cells, loss of cell viability could render the complete or partial loss of enzyme activity if rendered in air.^{4,10,24,25} In order to verify this and correlate the cell viability with product yield, we compared the photocatalytic ammonia turnover number carried out in air (contains dinitrogen and dioxygen) and pure dinitrogen (oxygen-free). Using respective ammonia yield performed in pure dinitrogen as a reference, no change of ammonia production was observed in air with nanorgs made from Au₂₂ NCs, while the ammonia turnover number with Au₁₈ NCs decreased to 30% of the yield in dinitrogen (FIG. 45A). The decrease of ammonia yield in air using nanorgs built from Au₁₈ NCs corresponds to the loss of cell viability, whose nitrogenase is partially inactivated by oxygen in air. Similar control experiments were performed in an argon atmosphere, and no detection of ammonia indicates the dinitrogen as the sole nitrogen source instead of the reaction media or the bacteria cellular components for ammonia production. Other control tests, including the removal of light, removal of cells, and Au NCs, were conducted to prove the generation of ammonia from light-driven enzymatic conversion in these nanorgs. To probe the effect of coupling chemistry between Au NCs and enzymes on nanorg biocatalytic conversion, we compared the light-driven ammonia production performance using the optimal Au₂₂ NCs with different A. vinelandii strains (FIG. 45b).

As mentioned previously, we selected the DJ995 strain that produces a nitrogenase with a 7× histidine tag in order to facilitate its coupling to the Au NCs (using Au-histidine interactions). As a comparison, a wild-type strain (A. vinelandii Wards) with no such affinity was also used to form the nanorgs. Under the same photocatalytic tests, their ammonia yield is half compared to our optimized systems, which could be explained by the poor electron transfer from the Au₂₂ to the nitrogenase due to unfavorable coupling between them. This again highlights the importance of (specific) coupling between the Au NCs and the enzyme for effective fuel production, as reported previously in the CdS/ZnS QDs-His-tagged nitrogenase biohybrids.¹⁰ Similar tests were also conducted on nanorgs made from A. vinelandii DJ1003 strain, which produces an apo-nitrogenase (also with histidine-tag) that lacks the essential Mo—Fe cofactors for nitrogen fixation. As expected, negligible amount of ammonia was produced.

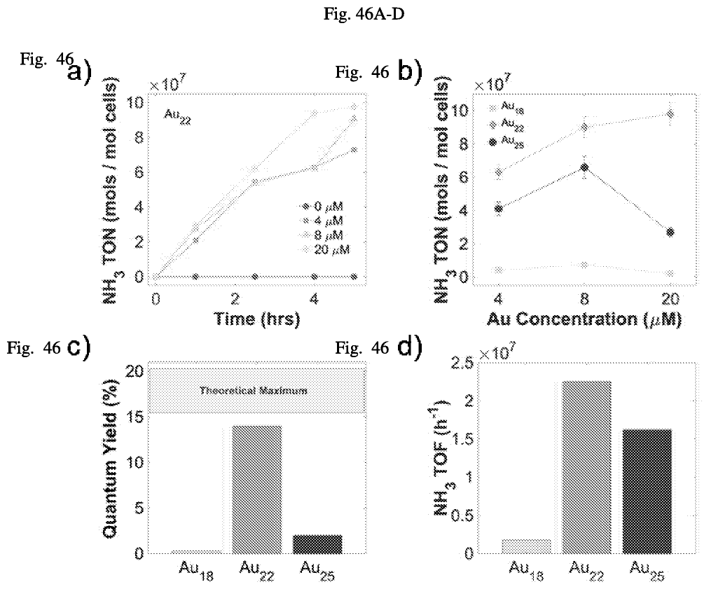

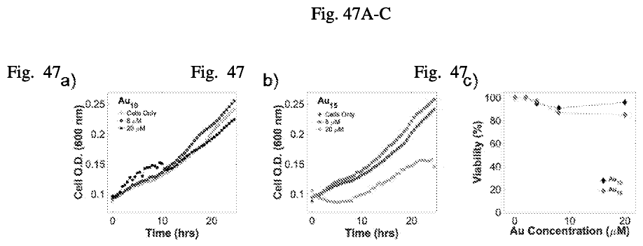

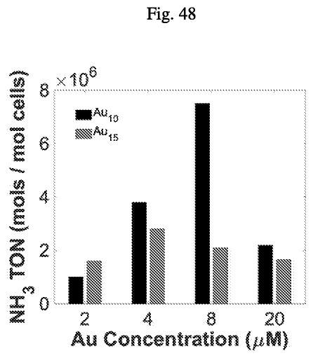

To optimize the turnover number, turnover frequency, and the photon-to-chemical conversion yield (quantum yield, QY), we conducted concentration-dependent light-driven air-water reduction reaction with different Au NCs-A. vinelandii nanorgs. Kinetics of ammonia production indicates no loss of nanorg viability or nitrogenase activity for more than 4 hours in Au₂₂ NC nanorgs (FIG. 46a), as shown in an almost linear increase of ammonia turnover number with time. The initial increase of ammonia turnover number with increasing Au₂₂ NCs concentration (FIG. 46b) can be explained by more efficient light capture and electron generation. However, as more Au₂₂ NCs are added, an insignificant increase of ammonia turnover number beyond 8 μM Au₂₂ NCs could be attributed to the intracellular nitrogenase bottleneck, where all available enzymes are coupled to the NCs. Similarly, nanorgs made from Au₂₅ NCs show initial increase of ammonia yield with NC concentration due to improved electron supply from light-activated NCs, while significant decrease of yield with 20 μM NCs is most likely caused by viability loss issues, which is also the reason of overall low ammonia yield for nanorgs made from Au₁₈ NCs. As for nanorgs made from Au₁₀₋₁₂ and Au₁₅ NCs, low ammonia yield (FIG. 48) could be explained by insufficient visible light absorption. Using the optimized conditions in nanorgs made from Au₁₈, Au₂₂, and Au₂₅ NCs, the highest ammonia turnover frequency (FIG. 46c) of >2×10⁷ h⁻¹ can be realized. To further understand the photon-to-chemical conversion efficiency, internal QY was calculated (FIG. 46d), by considering the total photon absorbed by the nanorgs and the final electrons resulted in ammonia generation. To estimate the maximum theoretical conversion yield, we estimated the photon flux per nano-biohybrid enzyme (˜5×10¹⁵ photons/sec/cm²), the nanorg density using cell OD (approximately 10⁸ nanorgs/cm²), and the number of biohybrid enzymes (approximately 10,000 biohybrid enzymes/nanorg), to obtain the photon flux per biohybrid enzymes (approximately 000 photons/biohybrid enzyme/sec). Comparing this to enzyme turnover (for MFN enzyme, 3000 nmol/mg/sec for 250 kDa enzyme≈750/sec), we estimated a maximum possible quantum efficiency to approximately 16-20% for photons-to-chemical generation using enzyme activation with light, limited by enzyme turnover rate.¹⁰ Therefore, this light-driven enzyme activity (zero in dark, and close to thermodynamic efficiency/enzyme turnover frequency on light irradiation) also provides a valuable tool to precisely control the enzyme activity of each enzyme individually using focused light (using either optics, or combined with multiphoton absorption in Au NCs/QDs), or controlling light intensity, thereby providing unprecedented control over single-enzyme sites for fine-tuning the enzyme activity, without changing the genetics/synthetic biology, or chemical changes to the cell. The highest QY of 14% (FIG. 46d) is very close to this theoretical maximum, indicating a highly efficient photon-to-fuel conversion system by combining efficacies of both high-efficiency light-sensitizer and high-selectivity enzymatic systems.

In conclusion, we have demonstrated light-driven biochemical catalysis using Au NC-A. vinelandii living nano-biohybrids (nanorgs), with a high turnover number (10⁸ mol/mol of cells) and turnover frequency (>2×10⁷ h⁻¹) for ammonia production in ambient air. By screening different atomically-precise Au NCs capped with zwitterionic ligand glutathione, we have selected the best candidate, Au₂₂ NCs, combining desired properties of high cellular uptake, effective light capture, suitable redox potential for electron transfer, and non-toxicity/biocompatibility, for building the nanorgs. In addition, the coupling of Au NCs to the His-tagged nitrogenase produced by A. vinelandii DJ995 strain demonstrates the importance of strong histidine-Au binding for high-efficiency biocatalytic applications. The optimized system can yield a high turnover frequency >2×10⁷ h⁻¹, with photon-to-chemical conversion efficiency (QY yield of 14%) approaching to the theoretical limit (16-20%). Since such high turnover number and frequency is difficult to achieve using conventional synthetic tools and such precise control over individual enzyme activity is not possible with most energetic substrates/conventional substrate-driven enzymatic catalysis, it could provide a new pathway for potentially better design of or high-throughput optimization of such biocatalytic reaction in optimized microbial systems.

These results can pave the way for expanding the choice of available benign and biocompatible materials, some with smaller sized light capturing/emitting particles than QDs, with high absorption cross-sections for use with optimized microbes, e.g. engineered, for building living nanorgs that can carry out light-driven biochemical catalysis for renewable solar fuel generation.

The use of such QDs and gold nanoclusters may also provide benefits over other systems for producing chemicals including reducing costs and lowering negative environmental impact of production.

Although various embodiments are specifically illustrated and described herein, it will be appreciated that modifications and variations of the present disclosure are covered by the above teachings, and as described herein, and are within the purview of the appended claims without departing from the spirit and intended scope of the disclosure. For example, while the engineered organisms are described herein for providing biofuel, such engineered organisms may also be useful for other applications such as plastics.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXPERIMENTAL

### Example I

### Synthesis of Glutathione-Capped Gold Nanoclusters (NCs)

Atomically-precise gold NCs capped with glutathione (GSH) capping ligands were synthesized based on the methods reported by Ghosh et al., Zhang et al., and Kamat et. al.¹¹⁻²⁰

Au₁₈ NCs were synthesized by dissolving 150 mg HAuCl₄3H₂O in 1.2 mL methanol and adding 1.8 mL DI Water. 300 mg of glutathione was added to this solution and sonicated to dissolve. Once the glutathione dissolved and the color changed from yellow to almost colorless. 96 mL methanol was then added and stirred for 10 min. 4.5 mL of a 220 mM NaBH₃CN solution was added under vigorous stirring for 30 min. After 30 min the precipitate was removed by centrifugation and washed with methanol repeatedly to remove any remaining precursor. Finally, the precipitate was dissolved in water and freeze-dried to obtain a pale red powder identified as Au₁₈SG₁₄.

Au₂₂ NCs were prepared by mixing 12.5 mL 20 mM HAuCl₄ and 7.5 mL 50 mM glutathione solution in a 500 mL flask containing 180 mL of DI water. After vigorously stirring for 2 min, the pH was raised to 12.0 with 1M NaOH, after which 0.24 mg NaBH₄ in 0.1 mL DI water was added to the reaction with sitting at 500 rpm. After 30 min, the pH was lowered to 2.5 with 0.33 M HCl. The reaction solution was then sealed airtight with stirring at 200 rpm and allowed to react for 8 hours forming a red-emitting Au₂₂ solution. The NCs were cleaned by using isopropyl alcohol and centrifugation and finally resuspended in water and kept in the fridge away from light for future use.

Au₁₀₋₁₂, Au₁₅, and Au₂₅ NCs were synthesized by carbon monoxide (CO) reducing techniques. Briefly, in a 125 ml flask with 20 ml distilled water, 100 mM HAuCl₄ and 200 mM reduced glutathione were added to a final concentration of 1 mM and 2 mM, respectively. The pH of the mixture was adjusted to 7, 9, and 11 for Au₁₀₋₁₂, Au₁₅, and Au₂₅ NCs synthesis, respectively. The flask was sealed with a rubber septum and flush with pure CO gas through a syringe needle for 2 min. The mixture was violently stirred for 24 hours and the resulting Au NCs was precipitated with an excess amount of isopropanol, followed by separating with centrifugation at 5,000 rpm. The precipitates were dried with clean air and re-suspend in distilled water. The Au NCs were stored in 4° C. for future tests.

### Example II

### Characterization of Au NCs

Ultraviolet-visible (UV-VIS) spectra were measured using the UV1600PC UV-VIS spectrometer (VWR). Dynamic light scattering (DLS) was performed on Litesizer 500 (Anton-Paar) to quantify the hydrodynamic size and zeta potential of the Au NCs.

The conduction band (CB) of the NCs was characterized using differential pulse voltammetry (DPV), with a Bio-logic SP200 potentiostat. A three-electrode configuration with a 3 mm glassy carbon working electrode, a platinum wire counter electrode, and a Ag/AgCl reference electrode was used. Au NCs suspension (in 0.1 M Na₂SO₄ electrolyte) was bubbled with argon for 10 min before the measurement. DPV was taken with the following parameters: 50 ms pulse width, 50 mV pulse height, 200 ms step width, and 4 mV step height (˜20 mV/s scan rate).

### Example III

### Preparation of A. vinelandii Culture

A. vinelandii DJ995 and DJ1003 strain were kindly provided by the Dennis group (Virginia Tech). Typically, bacteria were grown in nitrogen-limited Burk media (with 3 mM urea) at 30° C., with 200 rpm shaking. Cultures at an optical density (OD) ˜1.5 (overnight culture) were collected for future tests.

### Example IV

### Cell Growth Curve And Viability Tests

Cell growth curves and resazurin assays were performed in Burk media and photocatalytic media, respectively, with a variation of Au NC size. A. vinelandii DJ995 was first grown in nitrogen-free Burk media and harvested near OD 1.0, washed twice and resuspended in photocatalytic media. The cells and their respective NCs were then incubated and subject to light-exposure to produce NH₃. After production was complete (˜4 hrs) the nanorg mixtures were subject to both growth and viability measurements. The cell growth curves were taken in a 96-well microplate at 30 C with vigorous shaking and monitored using a microplate reader (TECAN GENios) controlled by Megellan 7.2 software. An initial OD of 0.1 in Burk media was used for the growth curves. The resazurin assay was performed separately in a 96-well plate with the bacteria maintained at an OD of 1.0 after the photocatalytic tests. Resazurin was added to a final concentration of 0.1 mg/mL and the fluorescence was measured at 620 nm (485 excitation) over the course of 2 hours.

### Example V

### Cellular Uptake

Uptake of Au NCs by A. vinelandii was determined by a UV-vis spectrophotometric method. NCs first had their UV-vis spectra recorded in photocatalytic media at the desired concentration (20 μM). The initial absorption value at the first excitonic peak was taken to be the 0% uptake mark (Absᵢ). Then the NCs at the desired concentration (20 μM) were incubated with the bacteria at OD 1.0 in photocatalytic media for 15 minutes on a shaker at 250 rpm. The mixtures were then centrifuged at 5,000 rpm and aliquots of the supernatant were taken for UV-vis measurement. The absorption values of the supernatant at the corresponding excitonic peak position (Abs_{f}) were used to calculate the uptake % using the following equation:

Uptake %=Abs₂Abs_{f}/Abs₁×100%

### Example VI

### Light-Driven Ammonia Production and Ammonia Assay

Ammonia production with the nanorgs was achieved by first growing A. vinelandii to an OD of ˜1.0 in Burk media. The cells were then washed twice with sugar-free photocatalytic media (25 mM ascorbic acid 35 mM HEPES) to remove any residual sugars. Cells were then resuspended in photocatalytic media and diluted to a final OD of 1.0. The Au NCs were then added to the bacteria and allowed to incubate for 15 min before subjecting to production. For production, the nanorgs were subjected to light irradiation (405 nm LED, 1.6 mW/cm²) for 5 hours on a shaker at 250 rpm. 25 μL aliquots were taken at various time points throughout the reaction to monitor the kinetics.

Ammonia was quantified using a fluorescent assay described previously. Typically, 25 μl sample was added to 0.5 ml o-phthalaldehyde assay reagent and incubated in dark for 30 min, followed by measuring the fluorescence at 472 nm (excitation at 410 nm).

### Example VII

### CdS and CdSe QDs: Nanoparticle (NP) Synthesis

CdS and CdSe nanoparticles for QDs were synthesized using a modified method developed by Peng et. al, (12/20). Their size can be controlled by varying the amount of oleic acid (OA) capping ligand. A total 12 g mixture containing 38.4 mg CdO, 316, 1904, or 5712 μl OA and 1-octadecene (ODE) was vacuum-degassed at 80° C. and refilled with argon for three cycles. The mixture was then heated up to 300° C. and injected with a sulfur precursor (4.8 mg sulfur powder dispersed in ODE). The resulting reaction phase was cooled down to 250° C. and the CdS QDs were grown for 1 hour. The removal of unconsumed cadmium and oleic acid was performed by extraction with warm CH₃OH (50° C.) in a separation funnel. This process was repeated three times and the resulting ODE layer was obtained, the removal of residual CH₃OH was carried out under vacuum at 80° C. To transfer the CdS nanoparticles to chloroform (CHCI3), the ODE layer was precipitated with chloroform and acetone followed by washing for at least three times. The CdS nanoparticles for QDs were re-dispersed in chloroform and stored in the dark. CdSe nanoparticles for QDs with varying sizes were synthesized using the same method, with the sulfur being replaced with a selenium precursor (12 mg selenium powder dispersed in ODE with 41 μl tributylphosphine (TBP)). Ultraviolet-visible (UV-VIS) spectra (FIG. 12A-F) were measured using the UVT600PC UV-VIS spectrometer (VWR).

### Example VIII

### InP@ZnS Core-Shell QDs (IPZS) Synthesis

IPZS were synthesized using a modified method developed by Fichter et. al, (26). A mixture of 9 ml oleylamine (OLA), 119.4 mg InCl₃, and 73.5 mg ZnCb were subjected to a vacuum, using a Schlenk system, at 110° C. for 1 h, followed by recharging with argon and ramping the temperature to 220° C. After 15 min, 0.24 ml tris(trimethylsilyl)phosphine ((TMS)₃P) was swiftly injected into the mixture, and the InP cores were allowed to continue growing for 10 min. The reaction phase was then cooled down to 80° C., and 1.27 ml of 1-dodecanethiol (DDT) was added drop-wise. The system was vacuum-degassed and recharged with argon, followed by raising the temperature to 200° C. for 1 hour. After cooling down to 70° C., 5 ml hexane was added, and the mixture was centrifuged at 4,000 rpm for 10 min to remove the large aggregates. The supernatant was precipitated with excess acetone, and the particles were obtained by centrifugation at 5,000 rpm for 20 min. After drying with N₂, the precipitate was suspended in 10 min OLA with 142.2 mg zinc stearate. The mixture was degassed and recharged with argon, and the temperature was raised to 180° C. After 3 hours, the system was cooled down, and the IPZS were obtained by centrifugation as mentioned above, and as described herein, and re-suspended in hexane.

### Example IX

### Cu₂ZnSnS₄@ZnS QDs (CZTS) Synthesis

CZTS QDs were synthesized following the procedure outlined by Yang et. al,^{S2} (27). Briefly, 0.13 g copper acetylacetonate, 0.05 g of zinc acetate dihydrate, 0.048 g of tin chloride, and 0.033 g of sulfur were added to 10 mL of oleylamine in a 40 mL round-bottom flask. The mixture was stirred under vacuum for 2 hours, then heated to 110° C. while purging with Ar and held for 30 min. The temperature was then raised to 280° C. and held for 1 hour, then cooled to room temperature. Precipitation of the nanoparticles was achieved by adding ethanol and centrifuging at 5,000 rpm for 20 min. Redispersion in chloroform and centrifugation at 5,000 rpm for 5 min was used to isolate and discard aggregates in the resulting pellet. ZnS shell growth follows the same protocols for the CdS or CdSe (see section D below, ZnS Shell Growth on CdS nanoparticle cores).

### Example X

### ZnS Shell Growth on Nanoparticle Cores

The synthesis (e.g., growing) of the CdS@ZnS core-shell nanoparticles for QDs (CZS) was adapted from the method reported by Peng et. al, (⁴⁴28). The size and concentration of the CdS cores (i.e. nanoparticles) were determined using the following formula:

D=(−6.6521×10⁻⁸)λ³+(1.9557×10⁻⁴)λ²−(9.2352×10⁻²)λ+13.29

ε5500×E×D^{2.5}

c=A/εl

where λ, E, A are the wavelength, photon energy, and extinction at the first exciton peak, respectively. l is the optical path of the cuvette. D, ε, and c are the diameter, extinction coefficient and concentration of the CdS nanoparticles for QDs. The CdS stock solution (dispersed in ODE) was determined to have a concentration of 0.0377 mM.

The zinc precursor (0.1 M Zn²⁺) was prepared by a heating a degassed mixture containing 82 mg ZnO, 2.82 ml (2.51 g) OA and 7.2 ml ODE to 250° C. The resulting clear solution was cooled down and stored in a septum sealed vial. The precursor was gently heated up to 60° C. before use.

The sulfur precursor (0.1 M) was prepared by dispersing 32 mg sulfur powder in 10 ml ODE with sonication. The resulting clear solution was bubbled with argon for 30 min and stored in a septum sealed vial.

ZnS shells were grown using a layer-by-layer deposition. A mixture containing 120 nmol CdS cores (3.55 nm; 3.2 ml CdS stock solution) and 2 ml oleylamine (OLA) was vacuum degassed and recharged with argon for three cycles under 120° C. A defined amount of zinc and sulfur precursors were injected simultaneously, and the reaction phase was kept at 120° C. for 5 min. The reaction was then raised up to 220° C. for the growth (20 min) of the first ZnS layer. The reaction was then cooled down to 120° C., and the UV-VIS spectrum was taken to determine the extinction coefficient of the CdS@ZnS core-shell nanoparticles (CZS). The resulting solution could be either washed (similar to CdS nanoparticles for QDs) or used for 2^{nd} or 3^{rd} ZnS layer growth. To grow nominal 1 approximately 3 monolayer ZnS shells, 0.44, 0.60 and 0.77 ml of zinc and sulfur precursors (each) in solvent were used, respectively, (determined by simple geometrical calculation, as reported by Peng et al.) zinc and sulfur precursors (each) were used, respectively.

The real thickness of CdS shell and extinction coefficient of CdS@ZnS core-shell nanoparticles (summarized in Table S1, Table S1-2) were determined by UV-VIS spectrum (FIG. 12C) and inductively coupled plasma mass spectrometry (ICP-MS), respectively.

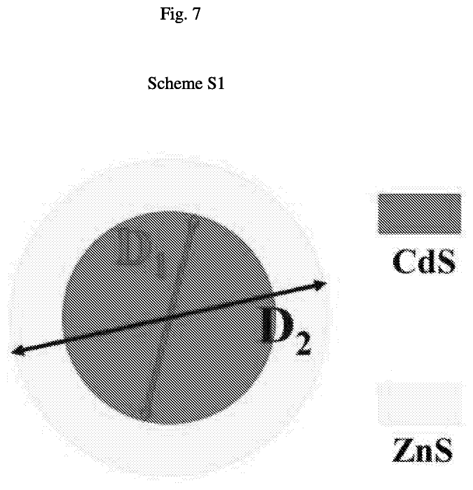

The real thickness of the ZnS coating can be estimated from the cadmium and zinc ratio (determined by ICP-MS) by using simple geometrical calculations shown in FIG. 7, Scheme 6.1, and described herein.

Similar to the CZS, CdSe@ZnS core-shell QDs (CZSe) can be synthesized using the same layer-by-layer deposition technique. Starting with CdSe QDs with the first exciton peak position at 500, 525 and 580 nm, CZSe1, CZSe2, and CZSe3 with green, yellow, and orange emission (FIG. 12A-F) can be synthesized with a nominal two monolayer ZnS shell.

### Example XI

### Ligand Exchange

QDs (nanoparticles) suspended in CHCl₃ were phase transferred into aqueous solution by ligand exchange with 3-mercaptopropionic acid (MPA), L-cysteine (CYS) or cysteamine (CA). For ligand exchange with MPA, 0.1 ml MPA was added to 0.3 ml QD suspension (approximately 10 mM). 0.3 ml ethanol (EtOH) was added, and the mixture was vigorously stirred with gentle heating. 1 ml 1 M NaOH solution was then added, and stirred for a further 5 min. The upper part (aqueous phase) was collected and centrifuged at 15,000 rpm. The obtained QD precipitates were re-suspended in water pH 11. The QD suspension was further concentrated with a 3,000 Da centrifugal filter and washed twice with water pH 11. The concentration of the QD suspension (in water) was determined using the UV-VIS spectra. Ligand exchange with CYS is similar. For the ligand exchange with CA, cysteamine hydrochloride was used, with the replacement of 1 M NaOH solution by DI water and finally re-suspended in water pH 4. The ligand-exchanged QDs (nanoparticle suspension) were stable for up to 1 week in the 4° C. fridge.

### Example XII

### Characterization of the QDs

A. Optical Spectroscopy.

Ultraviolet-visible spectra of the QDs were measured using the UV1600PC UV-VIS spectrometer (VWR), and the Photoluminescence (PL) spectra were taken using a QM-6 steady-state fluorimeter (PTI). The QDs optical spectra are shown in FIG. 12A-F.

B. Elemental Analysis

Real thickness of CdS shell and extinction coefficient of CdS@ZnS core-shell nanoparticles.

The real thickness of CdS shell and extinction coefficient of CdS@ZnS core-shell nanoparticles (summarized in Table S1, Table S1-2) were determined by UV-VIS spectrum (FIG. 12C) and inductively coupled plasma mass spectrometry (ICP-MS), respectively.

The real thickness of the ZnS coating can be estimated from the cadmium and zinc ratio (determined by ICP-MS) by using simple geometrical calculations shown in FIG. 7 Scheme 6.1. Elemental analysis with inductively coupled plasma mass spectrometry (ICP-MS) was used to determine the real thickness (Table S1) of the ZnS shell from the cadmium and zinc ratio, based on a simple geometrical calculation (FIG. 7 shows Scheme S1).

Taking the 1 ML sample as an example: The volume and moles of the CdS core (diameter D₁=3.55 nm, the density of CdS p(CdS)=4.82 g/cm³, MW is an approximate MW=molecular weight):

V(CdS)=π/6×D₁³=2.34×10⁻²⁰ cm³

n(CdS)=p(CdS)×V(CdS)/MW(CdS)=7.83×10⁻²² mol

The total amount of Cd and Zn (in ppb) was determined by ICP-MS. Due to Zn impurity in Cd precursor used in the synthesis, the value for the shell Zn was corrected by the amount of Zn in the CdS core (assume in a single nanoparticle, the amount of Cd is the same):

$$shell Zn ( 1 ML ) = total Zn ( 1 ML ) = - \frac{total Cd ( 1 ML )}{total Cd ( 0 ML )} \times total Zn ( 0 ML ) = 25390 ppb$$

The molar ratio of Zn to Cd (AW=approximate atomic weight):

$$r ( Zn : Cd ) = \frac{shell Zn ( 1 ML ) / AW ( Zn )}{total Cd ( 1 ML ) / AW ( Cd )} = 0.462$$

The moles and volume of the ZnS shell (the density of ZnS p(ZnS)=4.10 g/cm³):

n(ZnS)=r(Zn:Cd)×n(CdS)=3.62×10⁻²² mol

V(ZnS)=n(ZnS)×MW(ZnS)/p(ZnS)=0.860×10⁻²⁰ cm³

The total volume and diameter of the CdS @ZnS QDs:

V(CdS@ZnS)=V(CdS)+V(ZnS)=3.20×10⁻²⁰ cm³

D(CdS@ZnS)=(6×V(CdS@ZnS)/π)^{1/3}=3.94 nm

The real thickness (in ML unit, ZnS monolayer thickness d(ZnS)=0.312 nm):

Thickness (ZnS)=(D(CdS@ZnS)−D(CdS))/(2×d(ZnS))=0.63ML

The extinction coefficient (at a specific wavelength) of the QDs (core-shell, with nominal 2 monolayer ZnS shell) can be determined using the amount of QDs cores and the UV-VIS of the core-shell QDs. These values are summarized in Table S2.

C. Differential Pulse Voltammetry (DPV) Electrochemical Analysis

Differential pulse voltammetry (DPV) with QDs (in) suspension was used to determine the conduction and valence band positions of the CdX (X=S, Se) QDs. Refs. 27, 29, 45. This was done using a three-electrode configuration with a 2 mm platinum plate electrode, platinum wire, and silver wire as the working, counter, and (quasi-) reference electrode. Ferrocene was used as an internal reference. The CdX QDs (nanoparticles) were suspended in CH₂Cl₂ with 100 mM n-Bu₄NPF₆ as the electrolyte. The whole system was purged with argon, and the DPV was measured using a Bio-logic SP200 potentiostat with the following parameters: 50 ms pulse width, 50 mV pulse height, 200 ms step width and 4 mV step height (which corresponds to a 20 mV/s scan rate). The results are presented in FIG. 13A-D and the conduction/valence band (CB/VB) positions are listed The results are presented in FIG. 13A-D and the conduction/valence band (CB/VB) positions are listed in Table S3. The conduction/valence band (CB/VB) can be determined from the backward (cathodic) and forward (anodic) scan, respectively (FIG. 13A-D, Table S3). The same measurements were conducted on the other QDs.

D. CdS, CdSe, CdS(S>ZnS Nanoparticle Thin Film Electrochemistry.

QD thin film electrochemistry was conducted to evaluate the charge carrier dynamics of the QDs. The CdS, CdSe and CdS@ZnS nanoparticles (in ODE) were transferred into CHCI3, as described herein. 50 ul of about 5 uM suspension of nanoparticles were drop-casted on clean fluorinated-tin oxide (FTO) coated glass (about 0.5 cm²) and fully dried in a vacuum desiccator. The CdS, CdSe, and CdS@ZnS QD electrodes were fabricated by drop-casting 50 μl of 5 μM QDs suspension (in chloroform) on the clean fluorinated-tin oxide (FTO) coated glass slides (approximately 0.5 cm²).

Electrochemical measurements were taken using a three-electrode configuration, with the QD electrodes/nanoparticle coated FTO glass, platinum wire and Ag/AgCl electrode as a working, counter, and reference electrodes. 0.5 M sodium sulfate (pH=6.4) solution was used as an electrolyte. The whole system was purged with argon for 20 min before the measurements were taken.

Electrochemical impedance spectroscopy (EIS) was used to evaluate the charge trapping and charge transfer in these QDs under light irradiation. Measurements were taken under 365 nm UV irradiation (˜5 mW/cm²) at open circuit potential (OCP), with a frequency range from 100 kHz to 100 MHz. These spectra are shown with the Nyquist plot (FIG. 13E-G). Total capacitance (Ctotai) and space charge layer resistance (R_{sc}) can be extracted by fitting the spectra to the equivalent circuit described in FIG. 13H.

Open circuit potential (OCP) was used to evaluate the charge transfer and charge recombination on the QD surface. The OCP of the QD electrode was first measured in the dark, followed by irradiation with 365 nm UV light (˜5 mW/cm²) until the OCP reached constant values (FIG. 13i). The decay of OCP (with the “turn-off of the irradiation) was used to obtain the carrier lifetime (Table S4) regarding the surface charge recombination and charge injection to water, using a bi-exponential fit (Reference Nos. 29, 30, S4, S5): OCP (V)=A₁e^{-t/t1}+A₂e^{-t/t2} where t1 and t2 refer to the radiative/non-radiative charge recombination lifetime (faster) and the charge transfer lifetime (slower), respectively. And the coefficient (A₁ and A₂) indicates the relative fraction of these two carrier decay pathways. A significant loss of charge carriers (through surface recombination) were observed in the 0 ML and 1 ML samples due to the large A1/A2 ratio. For the 2 ML sample, there is still a significant number of charge carriers which end up transferring their charge to water.

E. Zeta Potential Measurements.

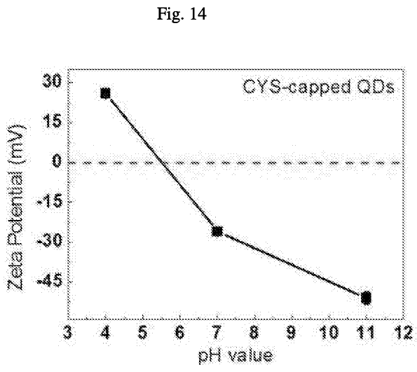

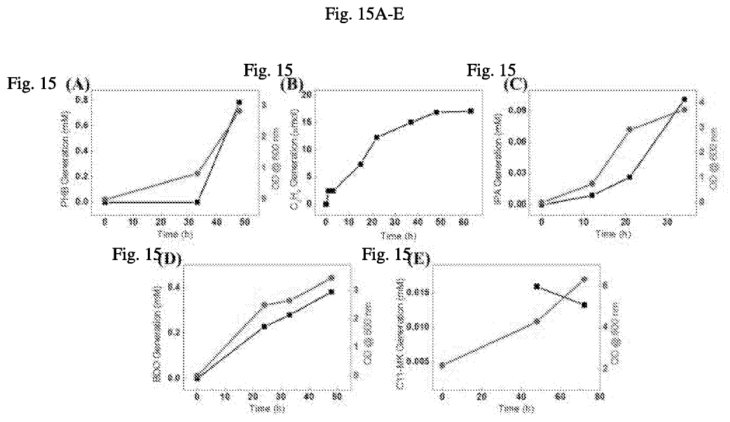

The zeta potential of the CYS-capped CZS (suspended in water with different pH values) was quantified using a Litesizer 500 (Anton-Paar). As expected (FIG. 14), CYS-capped QDs showed a decrease of zeta potential with increasing pH values, with the surface charge changing from positive to negative. The change in surface charge demonstrates the zwitterionic characteristic of cysteine, which could facilitate the incorporation of CYS-capped QDs by the cells.

### REFERENCES: SECTION I AND II

Each of which are herein incorporated by reference in its entirety:

A. Non-Parenthetical Numbers
1. Deisseroth, Optogenetics. Nat. Methods. 8, 26-9 (2011).
2. Khalil, Collins, Synthetic biology: Applications come of age. Nat. Rev. Genet. 11,367-379 (2010).
3. Brown et al, Light-driven dinitrogen reduction catalyzed by a CdS:nitrogenase MoFe protein biohybrid. Science. 352, 448-450 (2016).
4. Sakimoto, Wong, Yang, Self-photosensitization of nonphotosynthetic bacteria for solar-to-chemical production. Science. 351, 74-77 (2016).
5. Alivisatos, Semiconductor Clusters, Quantum Nanocrystals, and Quantum Dots. Science. 271, 933-937 (1996).
6. Reiss, M. Protiere, L. Li, Core/shell semiconductor nanocrystals. Small. 5, 154-168(2009).
7. Medintz, H. T. Uyeda, E. R. Goldman, H. Mattoussi, Quantum dot bioconjugates for imaging, labelling and sensing. Nat. Mater. 4, 435-446 (2005).
8. Choi et ah, Renal clearance of quantum dots. Nat. Biotechnol. 25, 1165-1170 (2007).
9. Jaiswal, Mattoussi, Mauro, Simon, Long-term multiple color imaging of live cells using quantum dot bioconjugates. Nat. Biotech. 21, 47-51 (2003).
10. Christiansen, Goodwin, Lanzilotta, Seefeldt, Dean, Catalytic and biophysical properties of a nitrogenase apo-MoFe protein produced by a nifB-deletion mutant of Azotobacter vinelandii. Biochemistry. 37, 12611-12623 (1998).
11. Milton et al, Nitrogenase bioelectrocatalysis: Heterogeneous ammonia and hydrogen production by MoFe protein. Energy Environ. Sci. 9, 2550-2554 (2016).
12. Yu, Peng, Formation of high-quality CdS and other II-VI semiconductor nanocrystals in noncoordinating solvents: Tunable reactivity of monomers. Angew. Chemie—Int. Ed. 41, 2368-2371 (2002).
13. Courtney et al., Photoexcited quantum dots for killing multidrug-resistant bacteria. Nat. Mater. 15, 529-534 (2016).
14. Goodman, Singh, Ribot, Chatterjee, Nagpal, Multiple energy exciton shelves in quantum-dot-DNA nanobioelectronics. /. Phys. Chem. Lett. 5, 3909-3913 (2014).
15. Goodman, A. Siu, V. Singh, P. Nagpal, Long-range energy transfer in self-assembled quantum dot-DNA cascades. Nanoscale. 7, 18435-18440 (2015).
16. Haram, B. M. Quinn, a J. Bard, Electrochemistry of CdS nanoparticles: a correlation between optical and electrochemical band gaps. /. Am. Chem. Soc. 123, 8860-8861 (2001).
17. Jasieniak, M. Califano, S. E. Watkins, in ACS Nano. 5, 5888-5902 (2011).
18. Amelia, C. Lincheneau, S. Silvi, A. Credi, Electrochemical properties of CdSe and CdTe quantum dots. Chem. Soc. Rev. 41, 5728 (2012).
19. Sundberg, Interactions of histidine and other imidazole derivatives with transition metal ions in chemical and biological systems. Chem. Rev. 74, 471-517 (1974).
20. Hardman, A toxicologic review of quantum dots: Toxicity depends on physicochemical and environmental factors. Environ. Health Perspect. 114, 165-172 (2006).
21. Yong et al, Nanotoxicity assessment of quantum dots: from cellular to primate studies. Chem. Soc. Rev. 42, 1236-1250 (2013).
22. Hoshino et al, Physicochemical properties and cellular toxicity of nanocrystal quantum dots depend on their surface modification. Nano Lett. 4, 2163-2169 (2004).
23. Lewinski, V. Colvin, R. Drezek, Cytotoxicity of nanopartides. Small. 4, 26-49 (2008).
24. Hoffman, D. Lukoyanov, Z. Y. Yang, D. R. Dean, L. C. Seefeldt, Mechanism of nitrogen fixation by nitrogenase: The next stage. Chem. Rev. 114, 4041-4062 (2014).
25. Eckert et al, Ethylene-forming enzyme and bioethylene production. Biotechnol. Biofuels. 7 (2014).

B. Parenthetical Numbers
(1) Deisseroth, K. Optogenetics. Nat. Methods 2011, 8 (1), 26-29.
(2) Khalil, A. S.; Collins, J. J. Synthetic Biology: Applications Come of Age. Nature Reviews Genetics. 2010, pp 367-379.
(3) Brown, K. A.; Harris, D. F.; Wilker, M. B.; Rasmussen, A.; Khadka, N.; Hamby, H.; Keable, S.; Dukovic, G.; Peters, J. W.; Seefeldt, L. C.; King, P. W. Light-Driven Dinitrogen Reduction Catalyzed by a CdS:nitrogenase MoFe Protein Biohybrid. Science. 2016, 352 (6284), 448-450.
(4) Milton, R. D.; Abdellaoui, S.; Khadka, N.; Dean, D. R.; Leech, D.; Seefeldt, L. C.; Minteer, S. D. Nitrogenase Bioelectrocatalysis: Heterogeneous Ammonia and Hydrogen Production by MoFe Protein. Energy Environ. Sci. 2016, 9 (8), 2550-2554.
(5) Milton, R. D.; Cai, R.; Sahin, S.; Abdellaoui, S.; Alkotaini, B.; Leech, D.; Minteer, S. D. The in Vivo Potential-Regulated Protective Protein of Nitrogenase in Azotobacter vinelandii Supports Aerobic Bioelectrochemical Dinitrogen Reduction in Vitro. J. Am. Chem. Soc. 2017, 139 (26), 9044-9052.
(6) Cai, R.; Milton, R. D.; Abdellaoui, S.; Park, T.; Patel, J.; Alkotaini, B.; Minteer, S. D. Electroenzymatic C—C Bond Formation from CO2. J. Am. Chem. Soc. 2018, 140 (15), 5041-5044.
(7) Sakimoto, K. K.; Wong, A. B.; Yang, P. Self-Photosensitization of Nonphotosynthetic Bacteria for Solar-to-Chemical Production. Science. 2016, 351 (6268), 74-77.
(8) Zhang, H.; Liu, H.; Tian, Z.; Lu, D.; Yu, Y.; Cestellos-Blanco, S.; Sakimoto, K. K.; Yang, P. Bacteria Photosensitized by Intracellular Gold Nanoclusters for Solar Fuel Production. Nature Nanotechnology. 2018, pp 900-905.
(9) Li, H.; Opgenorth, P. H.; Wernick, D. G.; Rogers, S.; Wu, T.-Y.; Higashide, W.; Malati, P.; Huo, Y.-X.; Cho, K. M.; Liao, J. C. Integrated Electromicrobial Conversion of C02 to Higher Alcohols. Science. 2012, 335 (6076), 1596-1596.
(10) Torella, J. P.; Gagliardi, C. J.; Chen, J. S.; Bediako, K. D.; Colón, B.; Way, J. C.; Silver, P. A.; Nocera, D. G. Efficient Solar-to-Fuels Production from a Hybrid Microbial-water-Splitting Catalyst System. Proc. Natl. Acad. Sci. 2015, 112 (8), 2337-2342.
(11) Liu, C.; Colón, B. C.; Ziesack, M.; Silver, P. A.; Nocera, D. G. Water Splitting-Biosynthetic System with C02 Reduction Efficiencies Exceeding Photosynthesis. Science. 2016, 352 (6290), 1210-1213.
(12) Liu, C.; Gallagher, J. J.; Sakimoto, K. K.; Nichols, E. M.; Chang, C. J.; Chang, M. C. Y.; Yang, P. Nanowire-Bacteria Hybrids for Unassisted Solar Carbon Dioxide Fixation to Value-Added Chemicals. Nano Lett. 2015, 15 (5), 3634-3639.
(13) Alivisatos, A. P. Semiconductor Clusters, Quantum Nanocrystals, and Quantum Dots. Science. 1996, 271 (5251), 933-937.
(14) Reiss, P.; Protière, M.; Li, L. Core/shell Semiconductor Nanocrystals. Small. 2009, pp 154-168.
(15) Medintz, I. L.; Uyeda, H. T.; Goldman, E. R.; Mattoussi, H. Quantum Dot Bioconjugates for Imaging, Labelling and Sensing. Nat. Mater. 2005, 4 (6), 435-446.
(16) Choi, H. S.; Liu, W.; Misra, P.; Tanaka, E.; Zimmer, J. P.; Ipe, B. I.; Bawendi, M. G.; Frangioni, J. V. Renal Clearance of Quantum Dots. Nat. Biotechnol. 2007, 25 (10), 1165-1170.
(17) Jaiswal, J. K.; Mattoussi, H.; Mauro, J. M.; Simon, S. M. Long-Term Multiple Color Imaging of Live Cells Using Quantum Dot Bioconjugates. Nat. Biotech. 2003, 21, 47-51.
(18) Christiansen, J.; Goodwin, P. J.; Lanzilotta, W. N.; Seefeldt, L. C.; Dean, D. R. Catalytic and Biophysical Properties of a Nitrogenase Apo-MoFe Protein Produced by a nifB-Deletion Mutant of Azotobacter vinelandii. Biochemistry 1998, 37 (36), 12611-12623.
(19) Milton, R. D.; Abdellaoui, S.; Khadka, N.; Dean, D. R.; Leech, D.; Seefeldt, L. C.; Minteer, S. D. Nitrogenase Bioelectrocatalysis: Heterogeneous Ammonia and Hydrogen Production by MoFe Protein. Energy Environ. Sci. 2016, 9 (8), 2550-2554.
(20) Yu, W. W.; Peng, X. Formation of High-Quality CdS and Other II-VI Semiconductor Nanocrystals in Noncoordinating Solvents: Tunable Reactivity of Monomers. Angew. Chemie—Int. Ed. 2002, 41 (13), 2368-2371.
(21) Courtney, C. M.; Goodman, S. M.; McDaniel, J. A.; Madinger, N. E.; Chatterjee, A.; Nagpal, P. Photoexcited Quantum Dots for Killing Multidrug-Resistant Bacteria. Nat. Mater. 2016, 15 (5), 529-534.
(22) Goodman, S. M.; Singh, V.; Ribot, J. C.; Chatterjee, A.; Nagpal, P. Multiple Energy Exciton Shelves in Quantum-Dot-DNA Nanobioelectronics. J. Phys. Chem. Lett. 2014, 5 (21), 3909-3913.
(23) Goodman, S. M.; Siu, A.; Singh, V.; Nagpal, P. Long-Range Energy Transfer in Self-Assembled Quantum Dot-DNA Cascades. Nanoscale 2015, 7 (44), 18435-18440.
(24) Haram, S. K.; Quinn, B. M.; Bard, a J. Electrochemistry of CdS Nanoparticles: A Correlation between Optical and Electrochemical Band Gaps. J. Am. Chem. Soc. 2001, 123, 8860-8861.
(25) Jasieniak, J.; Califano, M.; Watkins, S. E. Size-Dependent Valence and Conduction Band-Edge Energies of Semiconductor Nanocrystals. In ACS Nano; 2011; Vol. 5, pp 5888-5902.
(26) Amelia, M.; Lincheneau, C.; Silvi, S.; Credi, A. Electrochemical Properties of CdSe and CdTe Quantum Dots. Chem. Soc. Rev. 2012, 41 (17), 5728.
(27) Sundberg, R. J. Interactions of Histidine and Other Imidazole Derivatives with Transition Metal Ions in Chemical and Biological Systems. Chem. Rev. 1974, 74 (4), 471-517.
(28) Hardman, R. A Toxicologic Review of Quantum Dots: Toxicity Depends on Physicochemical and Environmental Factors. Environmental Health Perspectives. 2006, pp 165-172.
(29) Yong, K.-T. T.; Law, W.-C. C.; Hu, R.; Ye, L.; Liu, L.; Swihart, M. T.; Prasad, P. N. Nanotoxicity Assessment of Quantum Dots: From Cellular to Primate Studies. Chem. Soc. Rev. 2013, 42 (3), 1236-1250.
(30) Hoshino, A.; Fujioka, K.; Oku, T.; Suga, M.; Sasaki, Y. F.; Ohta, T.; Yasuhara, M.; Suzuki, K.; Yamamoto, K. Physicochemical Properties and Cellular Toxicity of Nanocrystal Quantum Dots Depend on Their Surface Modification. Nano Lett. 2004, 4 (11), 2163-2169.
(31) Lewinski, N.; Colvin, V.; Drezek, R. Cytotoxicity of Nanoparticles. Small 2008, 4 (1), 26-49.
(32) Hoffman, B. M.; Lukoyanov, D.; Yang, Z. Y.; Dean, D. R.; Seefeldt, L. C. Mechanism of Nitrogen Fixation by Nitrogenase: The next Stage. Chemical Reviews. 2014, 4041-4062.
(33) Eckert, C.; Xu, W.; Xiong, W.; Lynch, S.; Ungerer, J.; Tao, L.; Gill, R.; Maness, P. C.; Yu, J. Ethylene-Forming Enzyme and Bioethylene Production. Biotechnology for Biofuels. 2014, 7:33.
(34) Lovely, D. R. Electromicrobiology. Annu. Rev. Microbiol. 2012, 66, 391-409.

### REFERENCES: SECTION III

Each of which are herein incorporated by reference in it's entirety.
(1) Blankenship, R. E.; Tiede, D. M.; Barber, J.; Brudvig, G. W.; Fleming, G.; Ghirardi, M.; Gunner, M. R.; Junge, W.; Kramer, D. M.; Melis, A.; et al. Comparing Photosynthetic and Photovoltaic Efficiencies and Recognizing the Potential for Improvement. Science. 2011, pp 805-809.
(2) Appel, A. M.; Bercaw, J. E.; Bocarsly, A. B.; Dobbek, H.; Dubois, D. L.; Dupuis, M.; Ferry, J. G.; Fujita, E.; Hille, R.; Kenis, P. J. A.; et al. Frontiers, Opportunities, and Challenges in Biochemical and Chemical Catalysis of CO 2 Fixation. Chemical Reviews. 2013.
(3) Sakimoto, K. K.; Kornienko, N.; Yang, P. Cyborgian Material Design for Solar Fuel Production: The Emerging Photosynthetic Biohybrid Systems. Accounts of Chemical Research. 2017.
(4) Brown, K. A.; Harris, D. F.; Wilker, M. B.; Rasmussen, A.; Khadka, N.; Hamby, H.; Keable, S.; Dukovic, G.; Peters, J. W.; Seefeldt, L. C.; et al. Light-Driven Dinitrogen Reduction Catalyzed by a CdS:nitrogenase MoFe Protein Biohybrid. Science (80-.). 2016, 352 (6284), 448-450.
(5) Milton, R. D.; Abdellaoui, S.; Khadka, N.; Dean, D. R.; Leech, D.; Seefeldt, L. C.; Minteer, S. D. Nitrogenase Bioelectrocatalysis: Heterogeneous Ammonia and Hydrogen Production by MoFe Protein. Energy Environ. Sci. 2016, 9 (8), 2550-2554.
(6) Milton, R. D.; Cai, R.; Sahin, S.; Abdellaoui, S.; Alkotaini, B.; Leech, D.; Minteer, S. D. The in Vivo Potential-Regulated Protective Protein of Nitrogenase in Azotobacter vinelandii Supports Aerobic Bioelectrochemical Dinitrogen Reduction in Vitro. J. Am. Chem. Soc. 2017, 139 (26), 9044-9052.
(7) Cai, R.; Milton, R. D.; Abdellaoui, S.; Park, T.; Patel, J.; Alkotaini, B.; Minteer, S. D. Electroenzymatic C—C Bond Formation from CO2. J. Am. Chem. Soc. 2018, 140 (15), 5041-5044.
(8) Sakimoto, K. K.; Wong, A. B.; Yang, P. Self-Photosensitization of Nonphotosynthetic Bacteria for Solar-to-Chemical Production. Science (80-.). 2016, 351 (6268), 74-77.
(9) Zhang, H.; Liu, H.; Tian, Z.; Lu, D.; Yu, Y.; Cestellos-Blanco, S.; Sakimoto, K. K.; Yang, P. Bacteria Photosensitized by Intracellular Gold Nanoclusters for Solar Fuel Production. Nature Nanotechnology. 2018, pp 900-905.
(10) Ding, Y.; Bertram, J. R.; Eckert, C.; Bommareddy, R. R.; Patel, R.; Conradie, A.; Bryan, S. S.; Nagpal, P. Nanorg Microbial Factories: Light-Driven Renewable Biochemical Synthesis Using Quantum Dot-Bacteria Nanobiohybrids. J. Am. Chem. Soc. 2019, 141 (26), 10272-10282.
(11) Zheng, J.; Zhang, C.; Dickson, R. M. Highly Fluorescent, Water-Soluble, Size-Tunable Gold Quantum Dots. Phys. Rev. Lett. 2004.
(12) Zheng, J.; Nicovich, P. R.; Dickson, R. M. Highly Fluorescent Noble-Metal Quantum Dots. Annu. Rev. Phys. Chem. 2007.
(13) Chen, L. Y.; Wang, C. W.; Yuan, Z.; Chang, H. T. Fluorescent Gold Nanoclusters: Recent Advances in Sensing and Imaging. Analytical Chemistry. 2015.
(14) Dreaden, E. C.; Alkilany, A. M.; Huang, X.; Murphy, C. J.; El-Sayed, M. A. The Golden Age: Gold Nanoparticles for Biomedicine. Chemical Society Reviews. 2012.
(15) Shukla, R.; Bansal, V.; Chaudhary, M.; Basu, A.; Bhonde, R. R.; Sastry, M. Biocompatibility of Gold Nanoparticles and Their Endocytotic Fate inside the Cellular Compartment: A Microscopic Overview. Langmuir. 2005.
(16) Zhou, C.; Long, M.; Qin, Y.; Sun, X.; Zheng, J. Luminescent Gold Nanoparticles with Efficient Renal Clearance. Angew. Chemie—Int. Ed. 2011.
(17) Yu, Y.; Luo, Z.; Chevrier, D. M.; Leong, D. T.; Zhang, P.; Jiang, D. E.; Xie, J. Identification of a Highly Luminescent Au22(SG)18 Nanocluster. J. Am. Chem. Soc. 2014.
(18) Abbas, M. A.; Kamat, P. V.; Bang, J. H. Thiolated Gold Nanoclusters for Light Energy Conversion. ACS Energy Letters. 2018.
(19) Zhang, X. D.; Luo, Z.; Chen, J.; Song, S.; Yuan, X.; Shen, X.; Wang, H.; Sun, Y.; Gao, K.; Zhang, L.; et al. Ultrasmall Glutathione-Protected Gold Nanoclusters as next Generation Radiotherapy Sensitizers with High Tumor Uptake and High Renal Clearance. Sci. Rep. 2015.
(20) Mathew, A.; Pradeep, T. Noble Metal Clusters: Applications in Energy, Environment, and Biology. Part. Part. Syst. Charact. 2014.
(21) Choi, H. S.; Liu, W.; Misra, P.; Tanaka, E.; Zimmer, J. P.; Ipe, B. I.; Bawendi, M. G.; Frangioni, J. V. Renal Clearance of Quantum Dots. Nat. Biotechnol. 2007, 25 (10), 1165-1170.
(22) Goodman, S. M.; Levy, M.; Li, F.-F.; Ding, Y.; Courtney, C. M.; Chowdhury, P. P.; Erbse, A.; Chatterjee, A.; Nagpal, P. Designing Superoxide-Generating Quantum Dots for Selective Light-Activated Nanotherapy. Front. Chem. 2018, 6, 46.
(23) Levy, M.; Chowdhury, P. P.; Nagpal, P. Quantum Dot Therapeutics: A New Class of Radical Therapies. J. Biol. Eng. 2019, 13, 13:48.
(24) Hoffman, B. M.; Lukoyanov, D.; Yang, Z. Y.; Dean, D. R.; Seefeldt, L. C. Mechanism of Nitrogen Fixation by Nitrogenase: The next Stage. Chemical Reviews. 2014, pp 4041-4062.
(25) Christiansen, J.; Goodwin, P. J.; Lanzilotta, W. N.; Seefeldt, L. C.; Dean, D. R. Catalytic and Biophysical Properties of a Nitrogenase Apo-MoFe Protein Produced by a nifB-Deletion Mutant of Azotobacter vinelandii. Biochemistry 1998, 37 (36), 12611-12623.

### REFERENCES: SECTIONS IV-VI

Each of which are herein incorporated by reference in it's entirety:
1. Schmidt, M. Synthetic Biology: Industrial and Environmental Applications. (Wiley, 2012).
2. Nesbeth, D. N. Synthetic Biology Handbook. (CRC Press, 2016).
3. Postgate, J. Nitrogen Fixation. (Cambridge University Press, 1998).
4. Pandey, A. Biofuels: Alternative Feedstocks and Conversion Processes. (Academic Press, 2011).
5. Ho, D. P., Ngo, H. H. & Guo, W. A mini-review on renewable sources for biofuel. Bioresource Technology 169, 742-749 (2014).
6. Naik, S. N., Goud, V. V., Rout, P. K. & Dalai, A. K. Production of first and second generation biofuels: A comprehensive review. Renewable and Sustainable Energy Reviews 14, 578-597 (2010).
7. Roy, S. C, Varghese, O. K., Paulose, M. & Grimes, C. A. Toward solar fuels: Photocatalytic conversion of carbon dioxide to hydrocarbons. ACS Nano 4, 1259-1278 (2010).
8. Yilmaz, F., Balta, M. T. & Selbas, R. A review of solar based hydrogen production methods. Renewable and Sustainable Energy Reviews 56, 171-178 (2016).
9. Lewis, N. S. Toward cost-effective solar energy use. Science (80-.). 315, 798-802 (2007).
10. Seethamraju, S. et al. Materials and Processes for Solar Fuel Production. Materials and Processes for Solar Fuel Production 174, (2014).
11. Colmenares, J. C. & Xu, Y. Heterogeneous Photocatalysis, From Fundamentals to Green Applications. Chemical Reviews 93, (2016).
12. Gaya, U. I. Heterogeneous photocatalysis using inorganic semiconductor solids. Heterogeneous Photocatalysis Using Inorganic Semiconductor Solids 9789400777, (2014).
13. Vayssieres, L. On Solar Hydrogen & Nanotechnology. On Solar Hydrogen & Nanotechnology (2010). doi:10.1002/9780470823996
14. Tachibana, Y., Vayssieres, L. & Durrant, J. R. Artificial photosynthesis for solar water-splitting. Nat. Photonics 6, 511-518 (2012).
15. Habisreutinger, S. N., Schmidt-Mende, L. & Stolarczyk, J. K. Photocatalytic reduction of C02 on Ti02 and other semiconductors. Angewandte Chemie—International Edition 52, 7372-7408 (2013).
16. Hong, J., Zhang, W., Ren, J. & Xu, R. Photocatalytic reduction of C02: a brief review on product analysis and systematic methods. Anal. Methods 5, 1086 (2013).
17. Roger, I., Shipman, M. A. & Symes, M. D. Earth-abundant catalysts for electrochemical and photoelectrochemical water splitting. Nat. Rev. Chem. 1, 3 (2017).
18. Hunter, B. M., Gray, H. B. & Miiller, A. M. Earth-Abundant Heterogeneous Water Oxidation Catalysts. Chemical Reviews 116, 14120-14136 (2016).
19. Sakimoto, K. K., Wong, A. B. & Yang, P. Self-photosensitization of nonphotosynthetic bacteria for solar-to-chemical production. Science (80-.). 351,74-77 (2016).
20. Gupta, M. N., Jain, S. & Roy, I. Immobilized metal affinity chromatography without chelating ligands: Purification of soybean trypsin inhibitor on zinc alginate beads. Biotechnol. Prog. 18, 78-81 (2002).
21. Zhou, W. & Baneyx, F. Biofabrication of ZnS: Mnluminescent nanocrystals using histidine, hexahistidine, and His-tagged proteins: A comparison study. Biochem. Eng. J. 89,28-32(2014).
21. Ding, S. Y. et al. Bioconjugation of (CdSe)ZnS quantum dots using a genetically engineered multiple polyhistidine tagged cohesin/dockerin protein polymer. Macromol. Mater. Eng. 289, 622-628 (2004).
22. Dennis, A. M. et al. Surface ligand effects on metal-affinity coordination to quantum dots: Implications for nanoprobe self-assembly. Bioconjug. Chem. 21, 1160-1170 (2010).
23. Sapsford, K. E. et al. Kinetics of metal-affinity driven self-assembly between proteins or peptides and CdSe—ZnS quantum dots. J. Phys. Chem. C 111, 11528-11538 (2007).
24. Christiansen, J., Goodwin, P. J., Lanzilotta, W. N., Seefeldt, L. C. & Dean, D. R. Catalytic and biophysical properties of a nitrogenase apo-MoFe protein produced by a nifB-deletion mutant of Azotobacter vinelandii. Biochemistry 37, 12611-12623 (1998).
25. Ding, Y., Singh, V., Goodman, S. M. & Nagpal, P. Low exciton-phonon coupling, high charge carrier mobilities, and multiexciton properties in two-dimensional lead, silver, cadmium, and copper chalcogenide nanostructures. Journal of Physical Chemistry Letters 5, 4291-4297 (2014).
26. Bard, A. J. & Faulkner, L. R. Electrochemical Methods: Fundamentals and Applications, 2nd Edition. (John Wiley & Sons, 2000).
27. Lasia, A. Electrochemical Impedance Spectroscopy and its Applications. (Springer New York, 2014).
28. Memming, R. Semiconductor Electrochemistry. (Wiley, 2015).
29. Gao, H. et al. Ligand modified nanoparticles increases cell uptake, alters endocytosis and elevates glioma distribution and internalization. Sci. Rep. 3, 2534 (2013).
30. Chithrani, B. D., Ghazani, A. A. & Chan, W. C. W. Determining the size and shape dependence of gold nanoparticle uptake into mammalian cells. Nano Lett. 6, 662-668 (2006).
31. Yin Win, K. & Feng, S.-S. Effects of particle size and surface coating on cellular uptake of polymeric nanoparticles for oral delivery of anticancer drugs. Biomaterials 26,2713-2722(2005).
32. Nativo, P., Prior, I. A. & Brust, M. Uptake and intracellular fate of surface-modified gold nanoparticles. ACS Nano 2, 1639-1644 (2008).
33. Kong, B., Seog, J. H., Graham, L. M. & Lee, S. B. Experimental considerations on the cytotoxicity of nanoparticles. Nanomedicine 6, 929-941 (2011).
34. Bahadar, H., Maqbool, F., Niaz, K. & Abdollahi, M. Toxicity of nanoparticles and an overview of current experimental models. Iranian Biomedical Journal 20, 1-11 (2016).
35. Yildirimer, L., Thanh, N. T. K, Loizidou, M. & Seifalian, A. M. Toxicology and clinical potential of nanoparticles. Nano Today 6, 585-607 (2011).
36. Elsaesser, A. & Howard, C. V. Toxicology of nanoparticles. Advanced Drug Delivery Reviews 64, 129-137 (2012).
38. Lewinski, N., Colvin, V. & Drezek, R. Cytotoxicity of nanoparticles. Small 4,26-49 (2008).
39. Oberdorster, G., Stone, V. & Donaldson, K. Toxicology of nanoparticles: A historical perspective. Nanotoxicology 1, 2-25 (2007).
40. Turgeon, M. L. Clinical Hematology: Theory and Procedures. (Lippincott Williams & Wilkins, 2005).
41. Wintrobe, M. M. & Greer, J. P. Wintrobe's Clinical Hematology. (Wo Iters Kluwer Health/Lippincott Williams & Wilkins, 2009).
42. Celis, J. E. Cell Biology: A Laboratory Handbook. (Academic Press, 2005).
43. Yu, W. W. & Peng, X. Formation of high-quality CdS and other II-VI semiconductor nanocrystals in noncoordinating solvents: Tunable reactivity of monomers. Angew. Chemie—Int. Ed. 41, 2368-2371 (2002).
44. Chen, D., Zhao, F., Qi, H., Rutherford, M. & Peng, X. Bright and stable purple/blue emitting CdS/ZnS Core/Shell nanocrystals grown by thermal cycling using a single-source precursor. Chem. Mater. 22, 1437-1444 (2010).
45. Haram, S. K. et al. Quantum Confinement in CdTe Quantum Dots: Investigation through Cyclic Voltammetry Supported by Density Functional Theory (DFT). J. Phys. Chem. C 115, 6243-6249 (2011).
46. Baram, N. & Ein-Eli, Y. Electrochemical impedance spectroscopy of porous TiO₂ for photocatalytic applications. J. Phys. Chem. C 114, 9781-9790 (2010).
47. Kim, C. H., Newton, W. E. & Dean, D. R. Role of the MoFe Protein a-Subunit Histidine-195 Residue in FeMo-Cofactor Binding and Nitrogenase Catalysis. Biochemistry 34, 2798-2808 (1995).
48. Brown, K. A. et al. Light-driven dinitrogen reduction catalyzed by a CdS:nitrogenase MoFe protein biohybrid. Science (80-.). 352, 448-450 (2016).

### REFERENCES: SECTION VII

Each of which are herein incorporated by reference in it's entirety:
26. Ellis, M. A., Grandinetti, G. & Fichter, K. M. Synthesis of Cd-free InP/ZnS Quantum Dots Suitable for Biomedical Applications. J. Vis. Exp. 1-9 (2016).
27. Yang, H., Jauregui, L. A., Zhang, G., Chen, Y. P. & Wu, Y. Nontoxic and abundant copper zinc tin sulfide nanocrystals for potential high-temperature thermoelectric energy harvesting. Nano Lett. 12, 540-545 (2012).
28. Chen, D., Zhao, F., Qi, H., Rutherford, M. & Peng, X. Bright and stable purple/blue emitting CdS/ZnS Core/Shell nanocrystals grown by thermal cycling using a single-source precursor. Chem. Mater. 22, 1437-1444 (2010).
29. Baram, N. & Ein-Eli, Y. Electrochemical impedance spectroscopy of porous TiO2 for photocatalytic applications. J. Phys. Chem. C 114, 9781-9790 (2010).
30. Ding, Y. & Nagpal, P. Standalone anion- and co-doped titanium dioxide nanotubes for photocatalytic and photoelectrochemical solar-to-fuel conversion. Nanoscale 8, 17496-17505 (2016).
31. Goldman, E. & Green, L. H. Practical Handbook of Microbiology, Second Edition. (CRC Press, 2008).
32. Kim, C. H., Newton, W. E. & Dean, D. R. Role of the MoFe Protein a-Subunit Histidine-195 Residue in FeMo-Cofactor Binding and Nitrogenase Catalysis. Biochemistry 34, 2798-2808 (1995).
33. Müller et al., Engineering of Ralstonia eutropha H16 for autotrophic and heterotrophic production of methyl ketones. Appl. Environ. Microbiol. 79, 4433-4439 (2013).
34. Shah, V. K., Pate, J. L. & Brill, W. J. Protection of Nitrogenase in Azotobacter vinelandii. J. Bacteriol. 115, 15-17 (1973).
35. Ryu, K. S. Cho, E. G. Lee, Y. K. Chang, Recovery of poly(3-hydroxybutyrate) from coagulated Ralstonia eutropha using a chemical digestion method. Biotechnol. Prog. 16, 676-679 (2000).
S1. Ellis, M. A., Grandinetti, G. & Fichter, K. M. Synthesis of Cd-free InP/ZnS Quantum Dots Suitable for Biomedical Applications. J. Vis. Exp. 1-9 (2016).
S2. Yang, H., Jauregui, L. A., Zhang, G., Chen, Y. P. & Wu, Y. Nontoxic and abundant copper zinc tin sulfide nanocrystals for potential high-temperature thermoelectric energy harvesting. Nano Lett. 12, 540-545 (2012).
S3. Chen, D., Zhao, F., Qi, H., Rutherford, M. & Peng, X. Bright and stable purple/blue emitting CdS/ZnS Core/Shell nanocrystals grown by thermal cycling using a single-source precursor. Chem. Mater. 22, 1437-1444 (2010).
S4. Baram, N. & Ein-Eli, Y. Electrochemical impedance spectroscopy of porous TiO2 for photocatalytic applications. J. Phys. Chem. C 114, 9781-9790 (2010).
S5. Ding, Y. & Nagpal, P. Standalone anion- and co-doped titanium dioxide nanotubes for photocatalytic and photoelectrochemical solar-to-fuel conversion. Nanoscale 8, 17496-17505 (2016).
S6. Goldman, E. & Green, L. H. Practical Handbook of Microbiology, Second Edition. (CRC Press, 2008).
S7. Kim, C. H., Newton, W. E. & Dean, D. R. Role of the MoFe Protein a-Subunit Histidine-195 Residue in FeMo-Cofactor Binding and Nitrogenase Catalysis. Biochemistry 34, 2798-2808 (1995).
S8. Müller J., MacEachran D., Burd H., Sathitsuksanoh N., Bi C., Yeh Y. C., Lee T. S., Hillson N.J., Chhabra S. R., Singer S. W., Beller H. R. Engineering of Ralstonia eutropha H16 for autotrophic and heterotrophic production of methyl ketones. Appl. Environ. Microbiol. 79, 4433-4439(2013).
S9. Lynch S., Eckert C., Yu J., Gill R., Maness P.-C. Overcoming substrate limitations for improved production of ethylene in E. coli. Biotechnology for Biofuels. 9:3 (2016).
S10. Sievers F., Wilm A., Dineen D., Gibson T. J., Karplus K., Li W., Lopez R., McWilliam H., Remmert M., Soding J., Thompson J. D., Higgins D. G. Fast, scalable generation of high-quality protein multiple sequence alignments using Clustal Omega. Mol. Syst. Biol. 7:539 (2011).
S11. Brigham C. J., Budde C. F., Holder J. W., Zeng Q., Mahan A. E., Rha C., Sinskey A. J. Elucidation of beta-oxidation pathways in Ralstonia eutropha H16 by examination of global gene expression. Journal of Bacteriology. 192, 5454-5464 (2010)
S12. Shah, V. K., Pate, J. L. & Brill, W. J. Protection of Nitrogenase in Azotobacter vinelandii. J. Bacteriol. 115, 15-17 (1973).
S13. Ryu H. W., Cho K. S., Lee E. G., Chang Y. K., Recovery of poly(3-hydroxybutyrate) from coagulated Ralstonia eutropha using a chemical digestion method. Biotechnol. Prog. 16, 676-679 (2000).

1. An Azobacteria vinelandii bacteria strain DJ995 composition comprising a core-shell quantum dot (CZS-QD) having a Cadmium sulfide core and a two monolayer zinc sulfide (ZnS) shell (CdS@ZnS) surrounded by a zwitterion L-cysteine (CYS) cap, and a molybdenum-iron nitrogenase (MFN) enzyme.

2. The composition of claim 1, wherein said bacteria contains a compound in an amount above natural levels.

3. The composition of claim 2, wherein said compound is selected from the group consisting of ammonia (NH₃) molecules and hydrogen (H₂) molecule.

4. The composition of claim 2, wherein said compound is present in an amount greater than 10⁵ moles of compound per mole of bacteria cells.

5. A composition comprising a Cupriavidus necator bacteria strain comprising a Cadmium sulfide core and a two monolayer zinc sulfide (ZnS) shell (CdS@ZnS) core-shell quantum dot (CZS-QD) having a zwitterion L-cysteine (CYS) cap, and a molybdenum-iron nitrogenase (MFN) enzyme.

6. The composition of claim 4, wherein said Cupriavidus necator strain is an engineered strain comprising a pBBRl-efe plasmid.

7. A method of producing ammonia (NH₃), comprising,
a) providing,
i) a bacteria strain comprising a molybdenum-iron nitrogenase (MFN) enzyme having a reduction potential, wherein expression of said MFN enzyme results in ammonia (NH₃) formation, a plurality of (CZS) core-shell quantum dots (CZS-QDs), wherein said QD has a Cadmium sulfide core and a two monolayer zinc sulfide (ZnS) shell (CdS@ZnS) having a zwitterion L-cysteine (CYS) cap (CZS) core-shell quantum dot, wherein said CZS-QDs transmits electrons having energies in the range of the reduction potential of said MFN enzyme upon exposure to radiation that increases the activity of said MFN enzyme,
ii) an illumination source capable of emitting radiation,
iii) at least one compound selected from the group consisting of CO₂, H₂O, O₂ and N₂, and
b) incubating said engineered bacteria in the presence of said at least one said compound in the dark, and
c) irradiating said bacteria with said illumination source under conditions that produce a compound selected from the group consisting of ammonia (NH₃) and hydrogen (H₂) molecules.

8. The method of claim 7, wherein an amount of said compound is above natural levels.

9. The method of claim 7, wherein said production of said compound is an amount greater than in said bacteria strain without said QD.

10. The method of claim 7, wherein said production of said compound is an amount greater than in said bacteria strain without said irradiating.

11. The method of claim 7, wherein said production of said compound is an amount greater than 10⁵ moles of NH₃ per mole of said bacteria.

12. The method of claim 7, wherein said bacteria strain is an Azobacteria vinelandii bacteria strain DJ995.

13. The method of claim 7, wherein said bacteria strain is a Cupriavidus necator bacteria strain comprising a pBBRl-efe plasmid.

14. The method of claim 7, wherein said bacteria are live bacteria.

15. The method of claim 7, wherein said live bacteria are replicating.

16. A method of producing Polyhydroxybutyrate (PHB), comprising,
a) providing,
i) a bacteria strain comprising a molybdenum-iron nitrogenase (MFN) enzyme, wherein expression of said MFN enzyme results in Polyhydroxybutyrate (PHB) formation, a plurality of core-shell quantum dots (QDs) having a two monolayer zinc sulfide (ZnS) shell surrounded by a zwitterion L-cysteine (CYS) ligand cap, wherein said QD transmits electrons having energies in the range of the reduction potential of said MFN enzyme upon exposure to radiation that increases the activity of said MFN enzyme,
ii) an illumination source capable of emitting radiation, and
iii) at least one compound selected from the group consisting of CO₂, H₂O, O₂ and N₂,
b) incubating said engineered bacteria in the presence of said at least one compound in the dark, and
c) irradiating said engineered bacteria with said illumination source under conditions that produce Polyhydroxybutyrate (PHB) in an amount above natural levels.

17. The method of claim 16, wherein said QD is selected from the group consisting of a cadmium sulfide (CdS) core zinc sulfide (ZnS) shell QD CZS2 and a cadmium selenide (CdSe core zinc sulfide (ZnS) shell QD CZSe3.

18. The method of claim 16, wherein said production of PHB molecules is selected from the group consisting of an amount greater than in said bacteria strain without said QD; an amount greater than in said bacteria strain without said irradiation; an amount up to 100 mg of said PHB per gram of bacteria cell dry weight (CDW); an amount up to 150% of said natural levels.

19. The method of claim 16, wherein said production of PHB molecules is an amount greater than in said bacteria strain without said QD.

20. The method of claim 3, wherein said bacteria strain is selected from the group consisting of a Cupriavidus necator bacteria strain DJ995 comprising a pBBRl-yfp expression plasmid and an Azobacteria vinelandii bacteria strain comprising a pBBRl-yfp expression plasmid.
